(19) United States
(12) Patent Application Publication
(10) Pub. No.: US 2025/0221707 A1
(43) Pub. Date: Jul. 10, 2025

(54) END TOOL AND SURGICAL INSTRUMENT

(71) Applicant: LIVSMED INC., Seongnam-si (KR)

(72) Inventor: Woo Jung CHOI, Siheung-si (KR)

(21) Appl. No.: 19/007,903

(22) Filed: Jan. 2, 2025

(30) Foreign Application Priority Data
Jan. 4, 2024 (KR) 10-2024-0001317

(51) Int. Cl. A61B 17/072; A61B 17/00

(52) U.S. Cl. CPC A61B 17/072; A61B 2017/00367; A61B 2017/07271; A61B 2017/07285

(57) ABSTRACT

An end tool includes a jaw formed to accommodate at least one region of an operation member that is movable in at least one direction, a fixed pulley disposed closer to a distal end of the jaw than the operation member, and a forward-moving wire which is connected to the operation member after being rerouted in response to the fixed pulley to transmit a force to the operation member, and moves toward a proximal end of the jaw to transmit a driving force so that the operation member moves forward toward the distal end of the jaw.

### CROSS-REFERENCE TO RELATED APPLICATION

This application is based on and claims priority under 35 U.S.C. § 119 to Korean Patent Application No. 10-2024-0001317, filed on Jan. 4, 2024, in the Korean Intellectual Property Office, the disclosure of which is incorporated by reference herein in its entirety.

### BACKGROUND

### 1. Field

The present disclosure relates to an end tool and a surgical instrument.

### 2. Description of the Related Art

In recent years, laparoscopic surgery has been actively used to reduce postoperative recovery time and complications through small incisions. The laparoscopic surgery is a surgical method in which a plurality of small holes are drilled in the abdomen of a patient and the inside of the abdominal cavity is observed through these holes, and is widely used in general surgery and the like.

In performing such laparoscopic surgery, various instruments are being used. For example, suturing instruments that are inserted into the body to suture the surgical site within the abdominal cavity are used, and a surgical stapler that uses medical staples to suture the surgical site is used as the suturing instrument.

In general, a surgical stapler is a medical instrument that is often used for cutting and anastomosis of an organ in abdominal and thoracic surgery. The surgical stapler includes an open stapler used in thoracotomy and laparotomy and an endo stapler used in thoracoscopic surgery and celioscopic surgery.

The surgical stapler has advantages of not only shortening operation time since cutting of a surgical site and anastomosis of an organ are simultaneously performed, but also accurately suturing the surgical site. In addition, the surgical stapler has advantages of a faster recovery and a smaller scar than those when tissue is cut and sutured using a surgical stapling thread, and thus has been widely used in modern surgical operations. In particular, the surgical stapler has been widely used in cancer surgery to cut cancer tissue and suture a cut site.

### SUMMARY

The present disclosure is directed to providing an end tool and a surgical instrument capable of precisely moving an operation member during one or more steps involved in performing laparoscopic surgery or various other surgeries.

Additional aspects will be set forth in part in the description which follows and, in part, will be apparent from the description, or may be learned by practice of the presented embodiments of the disclosure.

An embodiment of the present disclosure discloses an end tool including a jaw formed to accommodate at least one region of an operation member that is movable in at least one direction, a fixed pulley disposed closer to a distal end of the jaw than the operation member, and a forward-moving wire which is connected to the operation member after being rerouted in response to the fixed pulley to transmit a force to the operation member, and of which at least one region moves toward a proximal end of the jaw to transmit a driving force so that the operation member moves forward toward the distal end of the jaw.

In the present embodiment, one region of the forward-moving wire may be connected and fixed to one region of a front surface of the operation member facing a forward-moving direction, and another region of the forward-moving wire may be disposed in a wire path that passes through front and rear sides of the operation member.

In the present embodiment, the wire path, through which the forward-moving wire passes, may be formed in a body of the operation member at a side portion protruding toward both sides and having a large width.

In the present embodiment, the fixed pulley may be disposed to be inclined, one region of the forward-moving wire may be connected to one region of a front surface of the operation member facing a forward-moving direction, and another region of the forward-moving wire may be disposed in one outer side of a body of the operation member.

In the present embodiment, when the operation member moves forward, one region of the forward-moving wire connected to the operation member and directed toward the fixed pulley may move toward the distal end of the jaw, and another region of the forward-moving wire, which is not fixed to the operation member and is directed to the fixed pulley, may move toward the proximal end of the jaw.

Another embodiment of the present disclosure discloses an end tool including a jaw formed to accommodate at least one region of an operation member that is movable in at least one direction, a fixed pulley disposed closer to a distal end of the jaw than the operation member, and a forward-moving wire which is disposed to be connected to a fixed region of the jaw after being rerouted in response to the fixed pulley and rerouted again by a support guide of the operation member, is configured to transmit a force to the operation member, and of which at least one region moves toward a proximal end of the jaw to transmit a driving force so that the operation member moves forward toward the distal end of the jaw.

In the present embodiment, the fixed region to which the forward-moving wire is fixed may be disposed between the fixed pulley and the operation member.

In the present embodiment, the fixed region may be disposed between a region of the forward-moving wire directed to the fixed pulley and a region of the forward-moving wire directed to the support guide of the operation member from the fixed pulley.

In the present embodiment, the forward-moving wire may pass through a wire path, which passes through front and rear sides of the operation member, and may be disposed to be directed the fixed pulley.

In the present embodiment, the support guide of the operation member may be formed on an inner side of the operation member, and after passing through the wire path and being directed toward the fixed pulley, the forward-moving wire may be rerouted through the fixed pulley to enter a first path of the operation member, and then emerge from a second path of the operation member after being correspondingly wound around the support guide to be fixed in the fixed region.

In the present embodiment, the fixed region may be disposed outside both a region of the forward-moving wire directed toward the fixed pulley and a region of the forward-moving wire directed toward the support guide of the operation member from the fixed pulley.

In the present embodiment, when the operation member moves forward, one region of the forward-moving wire, which is wound around the support guide and directed toward the fixed pulley, may move toward the distal end of the jaw, and another region of the forward-moving wire, which does not correspond to the support guide and is directed to the fixed pulley, may move toward the proximal end of the jaw.

In the present embodiment, the jaw may include a first jaw and a second jaw facing each other and formed to perform an opening and closing motion, wherein the first jaw may include an accommodation part formed to accommodate a plurality of staples, and the operation member may include a body, a blade region formed in one region of the body, and a first clamp and a second clamp each extending from the body and formed to have a width greater than that of the body, wherein the first clamp may pass through a guide groove of the second jaw and correspond to an upper surface of the second jaw, and the second clamp may pass through a guide groove of the first jaw and correspond to a lower surface of the first jaw.

In the present embodiment, the lower surface of the first jaw may include a protrusion and thus have a shape curved on both sides, the upper surface of the second jaw may include a protrusion and thus have a shape curved on both sides, the first clamp may be curved to correspond to the protrusion of the second jaw, and the second clamp may be curved to correspond to the protrusion of the first jaw.

In the present embodiment, the end tool may include a backward-moving wire connected to the operation member to enable the operation member to move backward in a direction opposite to a direction in which the operation member moves forward.

In the present embodiment, the backward-moving wire may include a portion disposed in parallel to the forward-moving wire with respect to the operation member in a vertical direction.

In the present embodiment, the backward-moving wire may be disposed on the same line as one region of the forward-moving wire extending from the operation member.

Another embodiment of the present disclosure discloses an end tool including a jaw formed to accommodate at least one region of an operation member that is movable in at least one direction, a plurality of fixed pulleys disposed closer to a distal end of the jaw than the operation member and spaced apart from each other, and a plurality of forward-moving wires which are connected to the operation member after being rerouted in response to the plurality of fixed pulleys, respectively, to transmit a force to the operation member, and of each of which at least one region moves toward a proximal end of the jaw to transmit a driving force so that the operation member moves forward toward the distal end of the jaw.

In the present embodiment, the forward-moving wires may include a first forward-moving wire and a second forward-moving wire, and the fixed pulleys may include a first fixed pulley and a second fixed pulley that are spaced apart from each other to correspond to the first forward-moving wire and the second forward-moving wire.

In the present embodiment, the first fixed pulley and the second fixed pulley may each be disposed to be inclined with an angle.

In the present embodiment, the angle formed by the first fixed pulley and the second fixed pulley may range from 70 degrees (deg) to 102 degrees (deg).

In the present embodiment, a rotation shaft of the first fixed pulley and a rotation shaft of the second fixed pulley may be formed not to be parallel to each other.

In the present embodiment, the first fixed pulley and the second fixed pulley may be disposed such that a gap therebetween is decreased in a downward direction with respect to a thickness direction of the jaw.

In the present embodiment, the jaw may include a first side surface and a second side surface, which have an inclined shape and face each other, and the first fixed pulley and the second fixed pulley may be disposed on the first side surface and the second side surface of the jaw, respectively.

In the present embodiment, the first fixed pulley and the second fixed pulley may be disposed on the first side surface and the second side surface of the jaw to be symmetrical to each other with respect to one center line of the jaw.

In the present embodiment, the first forward-moving wire and the second forward-moving wire may be disposed to be respectively connected to a first connection region and a second connection region, which are formed in the operation member at a region protruding outward from at least one surface.

In the present embodiment, the first forward-moving wire and the second forward-moving wire may extend from the proximal end of the jaw toward the distal end of the jaw, emerge from lower sides of the first fixed pulley and the second fixed pulley after being wound around upper sides of the first fixed pulley and the second fixed pulley, and may be connected to the first connection region and the second connection region, respectively.

In the present embodiment, a connection pulley may be disposed to be connected to the operation member and move together with the operation member, and each of the first forward-moving wire and the second forward-moving wire may be formed to be connected to the connection pulley.

In the present embodiment, a central axis of the connection pulley may be equal to or parallel to a height direction of a body of the operation member.

In the present embodiment, the connection pulley may be disposed in a groove formed on one surface of a body of the operation member, and when a cartridge is disposed in the jaw, the connection pulley may be disposed between a lower side surface of the cartridge and a bottom of the jaw.

In the present embodiment, the jaw may include a first jaw and a second jaw facing each other and formed to perform an opening and closing motion, the first jaw may include an accommodation part formed to accommodate a cartridge having a plurality of staples, and the first fixed pulley and the second fixed pulley may be disposed in a front space that is located closer to a distal end of the first jaw than the accommodation part of the first jaw.

In the present embodiment, the first fixed pulley and the second fixed pulley may be respectively disposed on a first side surface and a second side surface, which correspond to the front space and have a decreasing gap in a direction away from the second jaw.

In the present embodiment, the end tool may include a plurality of switching pulleys disposed closer to the proximal end of the jaw than the operation member to control paths of the first forward-moving wire and the second forward-moving wire.

In the present embodiment, the plurality of switching pulleys may include a first switching pulley configured to control the path of the first forward-moving wire and a second switching pulley configured to control the path of the second forward-moving wire, and the first switching pulley and the second switching pulley may be disposed around switching pulley shafts that are parallel to each other.

In the present embodiment, the first switching pulley and the second switching pulley may be disposed to be offset from each other rather than aligned on the same line with respect to a direction toward the operation member.

In the present embodiment, the plurality of switching pulleys may include a plurality of switching pulleys configured to control the path of the first forward-moving wire and a plurality of switching pulleys configured to the path of the second forward-moving wire, and the plurality of switching pulleys configured to control the path of the first forward-moving wire and the plurality of switching pulleys configured to control the path of the second forward-moving wire may be disposed on different switching pulley shafts.

In the present embodiment, the plurality of switching pulleys configured to control the path of the first forward-moving wire may include a third switching pulley and a fourth switching pulley disposed around switching pulley shafts that are parallel to each other, and the plurality of switching pulleys configured to control the path of the second forward-moving wire may include a first switching pulley and a second switching pulley disposed around switching pulley shafts that are parallel to each other.

In the present embodiment, the jaw may include a first side surface and a second side surface corresponding to a rear space that is located closer to the proximal end of the jaw than the operation member, the third switching pulley and the fourth switching pulley configured to control the path of the first forward-moving wire may be disposed on the first side surface, and the first switching pulley and the second switching pulley configured to control the path of the second forward-moving wire may be disposed on the second side surface,

In the present embodiment, the first side surface and the second side surface of the jaw may be formed such that a gap therebetween is decreased in a downward direction with respect to a thickness direction of the jaw.

In the present embodiment, the third switching pulley and the fourth switching pulley, which are disposed on the first side surface and configured to control the path of the first forward-moving wire and, may be disposed at positions with different heights, and the first switching pulley and the second switching pulley, which are disposed on the second side surface and configured to control the path of the second forward-moving wire, may be disposed at positions with different heights.

In the present embodiment, the end tool may include a backward-moving wire connected to the operation member to control a backward movement of the operation member, and a backward-moving wire switching pulley corresponding to a support shaft passing through the first side surface and the second side surface and configured to control a path of the backward-moving wire.

In the present embodiment, the backward-moving wire switching pulley may be disposed between the first side surface and the second side surface to be spaced apart from the first side surface and the second side surface.

In the present embodiment, the plurality of switching pulleys may be disposed closer to the operation member than a rotational motion shaft for performing one or more joint motions of the end tool.

In the present embodiment, the one or more joint motions may include a pitch motion or a yaw motion.

In the present embodiment, the forward-moving wires may include a first forward-moving wire and a second forward-moving wire, and when a cartridge is disposed in the jaw, at least one region of the first forward-moving wire and the second forward-moving wire may include regions that respectively correspond to spaces between both side surfaces of the jaw and the cartridge.

In the present embodiment, one regions of the first forward-moving wire and the second forward-moving wire emerging from being wound around of a lower side of the fixed pulley may be disposed on a lower side of the cartridge, and one regions of the first forward-moving wire and the second forward-moving wire emerging from being wound around an upper side of the fixed pulley may be disposed on respectively both sides between the jaw and the cartridge.

In the present embodiment, when the operation member moves forward, one region of the forward-moving wire, which is connected to the operation member and directed toward the fixed pulley, may move toward the distal end of the jaw, and another region of the forward-moving wire, which is not fixed to the operation member and is directed to the fixed pulley, may move toward the proximal end of the jaw.

Another embodiment of the present disclosure discloses an end tool including a jaw formed to accommodate at least one region of an operation member that is movable in at least one direction, a first rotation shaft pulley disposed to allow the jaw to rotationally move around one shaft, a second rotation shaft pulley disposed to allow the jaw to rotationally move around another shaft that is different from the one shaft around which the first rotation shaft pulley rotationally moves, and one or more switching pulleys formed to control paths of one or more forward-moving wires that are configure to transmit a driving force to cause the operation member to move, and disposed closer to the operation member than at least the first rotation shaft pulley and the second rotation shaft pulley.

In the present embodiment, the first rotation shaft pulley may be a yaw pulley corresponding to a yaw motion of the end tool, the second rotation shaft pulley may be a pitch pulley corresponding to a pitch motion of the end tool, and the first rotation shaft pulley may be disposed closer to the switching pulleys than the second rotation shaft pulley.

In the present embodiment, the forward-moving wires may include a first forward-moving wire and a second forward-moving wire, the switching pulleys may include a first switching pulley and a second switching pulley corresponding to the first forward-moving wire and the second forward-moving wire, respectively, and the first switching pulley and the second switching pulley may be disposed parallel to each other.

In the present embodiment, the end tool may include a first auxiliary pitch pulley disposed adjacent to a pitch pulley, which is the second rotation shaft pulley, having an axis parallel to that of the second rotation shaft pulley, and configured to control paths of the first forward-moving wire and the second forward-moving wire.

In the present embodiment, the end tool may include a yaw auxiliary pitch pulley disposed between the first auxiliary pitch pulley and the first rotation shaft pulley, having an axis parallel to that of the first rotation shaft pulley, and configured to control the paths of the first forward-moving wire and the second forward-moving wire.

In the present embodiment, the end tool may include a second auxiliary pitch pulley disposed on a side of a pitch pulley, which is opposite to a side thereof in which the first auxiliary pitch pulley is disposed, having an axis parallel to that of the second rotation shaft pulley, and configured to control the paths of the first forward-moving wire and the second forward-moving wire, wherein the pitch pulley is the second rotation shaft pulley.

In the present embodiment, paths of the first forward-moving wire and the second forward-moving wire may be controlled through the first switching pulley and the second switching pulley such that the first forward-moving wire and the second forward-moving wire are gathered to be correspondingly in contact with the same side of at least the first rotation shaft pulley.

In the present embodiment, the end tool may include a backward movement wire for backward movement of the operation member, wherein, one or more backward-moving wire switching pulleys configured to control a path of the backward-moving wire may be disposed on a shaft identical to that of the first switching pulley or the second switching pulley.

In the present embodiment, the first rotation shaft pulley may be a pitch pulley corresponding to a pitch motion of the end tool, the second rotation shaft pulley may be a yaw pulley corresponding to a yaw motion of the end tool, and the first rotation shaft pulley may be disposed closer to the switching pulley than the second rotation shaft pulley.

In the present embodiment, the forward-moving wires may include a first forward-moving wire and a second forward-moving wire, and the switching pulleys may include a third switching pulley and a fourth switching pulley corresponding to the first forward-moving wire, and a first switching pulley and a second switching pulley corresponding to the second forward-moving wire.

In the present embodiment, a central axis corresponding to the third switching pulley and a central axis corresponding to the fourth switching pulley may be parallel to each other, a central axis corresponding to the first switching pulley and a central axis corresponding to the second switching pulley may be parallel to each other, and the central axis corresponding to the third switching pulley and the central axis corresponding to the fourth switching pulley may respectively intersect the central axis corresponding to the first switching pulley and the central axis corresponding to the second switching pulley.

In the present embodiment, the third switching pulley and the fourth switching pulley may be disposed on the same plane, the first switching pulley and the second switching pulley may be disposed on the same plane, and the plane on which the third switching pulley and the fourth switching pulley are disposed and the plane on which the first switching pulley and the second switching pulley are disposed may be disposed to be inclined to each other.

In the present embodiment, the third switching pulley and the fourth switching pulley may be disposed on the same plane at positions with different heights, and the first switching pulley and the first switching pulley may be disposed on the same plane at positions with different heights.

In the present embodiment, the third switching pulley and the fourth switching pulley may be disposed to be symmetrical to the first switching pulley and the second switching pulley.

In the present embodiment, the end tool may include an auxiliary pitch pulley disposed adjacent to a pitch pulley, which is the first rotation shaft pulley, having an axis parallel to that of the first rotation shaft pulley, and configured to control paths of the first forward-moving wire and the second forward-moving wire.

In the present embodiment, the end tool may include a first auxiliary yaw pulley disposed between the auxiliary pitch pulley and the second rotation shaft pulley, having an axis parallel to that of the second rotation shaft pulley, and configured to control the paths of the first forward-moving wire and the second forward-moving wire.

In the present embodiment, the end tool may include a second auxiliary yaw pulley disposed on a side of a yaw pulley, which is opposite to a side thereof in which the first auxiliary yaw pulley is disposed, having an axis parallel to that of the second rotation shaft pulley, and configured to control the paths of the first forward-moving wire and the second forward-moving wire, wherein the pitch pulley is the second rotation shaft pulley.

In the present embodiment, the end tool may include a backward-moving wire switching pulley disposed to correspond to a support shaft passing through a plane, on which the third switching pulley and the fourth switching pulley are formed, and a plane, on which the first switching pulley and the second switching pulley are formed, to control a path for backward movement of the operation member.

Another embodiment of the present disclosure discloses a surgical instrument including any of the above-described end tools, an operator configured to control an operation of the end tool, and a connector configured to connect the operator to the end tool.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic perspective view of a surgical instrument according to an embodiment of the present disclosure;

FIG. 2 is a schematic perspective view for describing an end tool of FIG. 1;

FIG. 3 is a schematic perspective view of the end tool of FIG. 2 with a second jaw removed;

FIG. 4 is a schematic perspective view of the end tool of FIG. 3 with a cartridge removed;

FIG. 5 is a plan view schematically illustrating the second jaw of the end tool of FIG. 2;

FIG. 6 is a perspective view schematically illustrating the second jaw of the end tool of FIG. 2;

FIG. 7 is a perspective view schematically illustrating a first jaw of the end tool of FIG. 2;

FIG. 8 is a plan view schematically illustrating the first jaw of the end tool of FIG. 2;

FIG. 9 is a perspective view illustrating an operation member of the end tool of FIG. 2;

FIG. 10 is a perspective view of the operation member of FIG. 9 viewed from another direction;

FIG. 11 is a side view of the operation member of FIG. 9 viewed in one direction;

FIG. 12 is a front view of the operation member of FIG. 9 viewed in one direction;

FIG. 13 is a perspective view illustrating a wedge of the end tool of FIG. 2;

FIG. 14 is a schematic perspective view for describing the operation member, a fixed pulley, and a forward-moving wire of the end tool of FIG. 2;

FIG. 15 is a bottom view of FIG. 14 viewed in one direction;

FIG. 16 is a perspective view illustrating a state in which the first jaw is removed from FIG. 14;

FIG. 17 is a plan view of FIG. 16 viewed in one direction;

FIG. 18 is a front view of FIG. 16 viewed in one direction;

FIGS. 19A, 19B, and 19C are schematic perspective views for describing an operation of the operation member of the end tool of FIG. 2;

FIGS. 20A, 20B, and 20C are schematic side views for describing an operation of the operation member of the end tool of FIG. 2;

FIGS. 21 to 24 are views for describing one modified example of the end tool of FIG. 2;

FIGS. 25A, 25B, and 25C are views for describing an optional embodiment in which a backward-moving wire is added to the end tool of FIG. 2;

FIG. 26 is a front view of the end tool of FIG. 2 viewed in one direction;

FIG. 27 is a view illustrating another modified example of the end tool of FIG. 2;

FIG. 28 is a perspective view illustrating the first jaw and the cartridge of the surgical instrument of FIG. 1;

FIGS. 29A, 29B, 29C, and 30 are cross-sectional views illustrating a staple motion of the end tool of the surgical instrument of FIG. 1 as a whole;

FIG. 31 is a perspective view schematically illustrating an end tool of a surgical instrument according to another embodiment of the present disclosure;

FIG. 32 is a schematic perspective view of the end tool of FIG. 31 with a second jaw removed;

FIG. 33 is a schematic perspective view of the end tool of FIG. 32 with a cartridge removed;

FIG. 34 is a perspective view schematically illustrating a first jaw of the end tool of FIG. 31;

FIG. 35 is a plan view of FIG. 34 viewed in one direction;

FIG. 36 is a front view of FIG. 34 viewed in one direction;

FIG. 37 is a perspective view illustrating an operation member of the end tool of FIG. 31;

FIG. 38 is a perspective view of the operation member of FIG. 37 viewed from another direction;

FIG. 39 is a perspective view for describing a support guide of the operation member of FIG. 37;

FIG. 40 is a perspective view illustrating a wedge of the end tool of FIG. 31;

FIG. 41 is a front view of FIG. 40;

FIG. 42 is a schematic perspective view for describing the operation member, a fixed pulley, and a forward-moving wire of the end tool of FIG. 31;

FIG. 43 is a schematic perspective view illustrating a state in which the first jaw is removed from FIG. 42;

FIG. 44 is a side view of FIG. 43 viewed in one direction;

FIGS. 45 and 46 illustrate enlarged views of different regions of FIG. 44, respectively;

FIGS. 47A, 47B, and 47C are schematic perspective views for describing an operation of the operation member of the end tool of FIG. 31;

FIGS. 48A, 48B, and 48C are views for describing an optional embodiment in which a backward-moving wire is added to the end tool of FIG. 31;

FIG. 49 is a view for describing one modified example of the end tool of FIG. 31;

FIG. 50 is a schematic perspective view of a surgical instrument according to another embodiment of the present disclosure;

FIG. 51 is a schematic perspective view for describing an end tool of FIG. 50;

FIG. 52 is a perspective view of the end tool of FIG. 51 viewed from another direction.

FIG. 53 is a schematic perspective view of the end tool of FIG. 51 with a second jaw removed;

FIG. 54 is a schematic perspective view of the end tool of FIG. 53 with a cartridge removed;

FIG. 55 is a transparent perspective view of FIG. 54;

FIG. 56 is a perspective view schematically illustrating the second jaw of the end tool of FIG. 51;

FIG. 57 is a perspective view schematically illustrating a first jaw of the end tool of FIG. 51;

FIG. 58 is a plan view schematically illustrating the first jaw of the end tool of FIG. 51;

FIG. 59 is a perspective view illustrating an operation member of the end tool of FIG. 51;

FIG. 60 is a perspective view of the operation member of FIG. 59 viewed from another direction;

FIG. 61 is a front view of the operation member of FIG. 59 viewed in one direction;

FIG. 62 is a schematic perspective view illustrating a portion of the end tool of FIG. 51;

FIG. 63 is a front view of FIG. 62 viewed in one direction;

FIG. 64 is a schematic plan view for describing the operation member, a fixed pulley, and a forward-moving wire of the end tool of FIG. 51;

FIG. 65 is a schematic perspective view for describing the operation member, the fixed pulley, and the forward-moving wire of the end tool of FIG. 51;

FIG. 66 and FIGS. 67A, 67B, and 67C are schematic views for describing an operation of the operation member of the end tool of FIG. 51;

FIG. 68 and FIGS. 69A, 69B, and 69C are views for describing an optional embodiment in which a backward-moving wire is added to the end tool of FIG. 51;

FIG. 70 is a perspective view illustrating the first jaw and the cartridge of the surgical instrument of FIG. 50;

FIGS. 71 and 72 are views for describing a switching pulley, a yaw pulley, and a pitch pulley of the end tool of the surgical instrument of FIG. 50;

FIG. 73 is a schematic perspective view illustrating an end tool of a surgical instrument according to another embodiment of the present disclosure;

FIG. 74 is a perspective view of the end tool of FIG. 73 viewed from another direction;

FIG. 75 is a schematic perspective view of the end tool of FIG. 73 with a second jaw removed;

FIG. 76 is a schematic perspective view of the end tool of FIG. 75 with a cartridge removed;

FIG. 77 is a partial perspective view illustrating one region of FIG. 76;

FIG. 78 is a perspective view schematically illustrating the second jaw of the end tool of

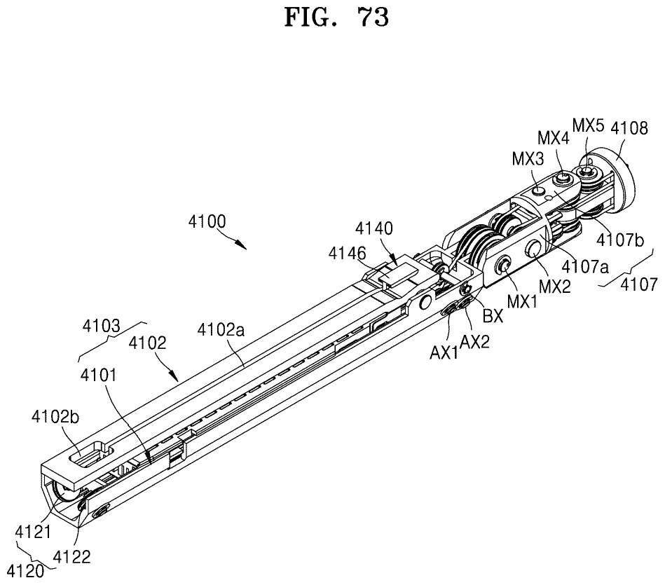

FIG. 73;

FIG. 79 is a perspective view schematically illustrating a first jaw of the end tool of FIG. 73.

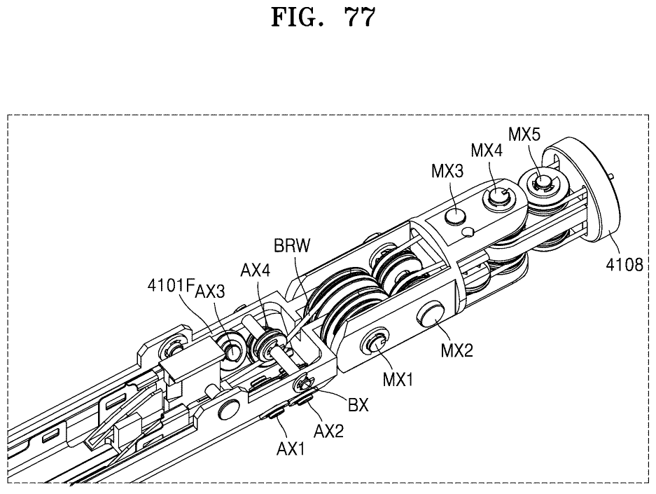

FIG. 80 is a schematic plan view illustrating a portion of the end tool of FIG. 77;

FIG. 81 is a magnified view of a portion of FIG. 80;

FIG. 82 is a schematic perspective view for describing an operation member, a fixed pulley, and a forward-moving wire of the end tool of FIG. 73;

FIG. 83 is a plan view of FIG. 82;

FIG. 84 and FIGS. 85A, 85B, and 85C are schematic views for describing an operation of the operation member of the end tool of FIG. 73;

FIG. 86 and FIGS. 87A, 87B, and 87C are views for describing an optional embodiment in which a backward-moving wire is added to the end tool of FIG. 73;

FIGS. 88 and 89 are views for describing a switching pulley, a yaw pulley, and a pitch pulley of the end tool of the surgical instrument of FIG. 73;

FIG. 90 is a schematic perspective view illustrating an end tool of a surgical instrument according to another embodiment of the present disclosure;

FIG. 91 is a perspective view schematically illustrating an operation member disposed in the end tool of FIG. 90;

FIG. 92 is a schematic perspective view for describing the operation member, a fixed pulley, and a forward-moving wire of the end tool of FIG. 90;

FIG. 93 is a magnified view illustrating one region of FIG. 92;

FIG. 94 is a plan view of FIG. 92;

FIG. 95 and FIGS. 96A, 96B, and 96C are schematic views for describing an operation of the operation member of the end tool of FIG. 90;

FIG. 97 and FIGS. 98A, 98B, and 98C are views for describing an optional embodiment in which a backward-moving wire is added to the end tool of FIG. 90;

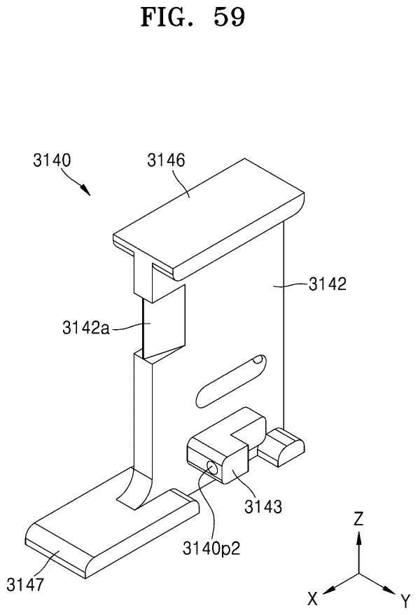

FIG. 99 is a perspective view illustrating one modified example of the operation member of FIG. 59;

FIG. 100 is a perspective view illustrating the operation member in a direction different from that of FIG. 99;

FIG. 101 is a perspective view illustrating a state in which a forward-moving wire and a backward-moving wire are connected to the operation member of FIG. 99;

FIG. 102 is a perspective view schematically illustrating a surgical instrument according to another embodiment of the present disclosure;

FIG. 103 is a side view of the surgical instrument of FIG. 102; and

FIGS. 104A and 104B are diagrams for schematically describing an operation concept of the surgical instrument of FIG. 102.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

While the present disclosure is susceptible to various modifications and alternative forms, specific embodiments thereof are shown by way of example in the drawings and will herein be described in detail. Advantages and features of the present disclosure and methods for accomplishing the same will be more clearly understood from embodiments described below with reference to the drawings. However, the present disclosure is not limited to the embodiments disclosed below but may be implemented in various forms.

Hereinafter, the embodiments of the present disclosure will be described in detail with reference to the accompanying drawings, but when describing with reference to the drawings, equal or corresponding components will be referred to as the same reference numerals, and redundant descriptions thereof will be omitted.

In the following embodiments, the terms “first,” “second,” and the like have been used to distinguish one component from another, rather than limitative in all aspects.

In the following embodiments, singular forms are intended to include plural forms as well, unless the context clearly indicates otherwise.

In the following embodiments, terms such as “include” or “have” means that the features or components described in the specification are present, and the possibility that one or more other features or components will be added is not excluded in advance.

Sizes of components in the drawings may be exaggerated or reduced for convenience of description. For example, the size and thickness of each component shown in the drawings are arbitrarily represented for convenience of description, and thus, the present disclosure is not necessarily limited thereto.

In the following embodiments, an x-axis, a y-axis, and a z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in an order opposite to the described order.

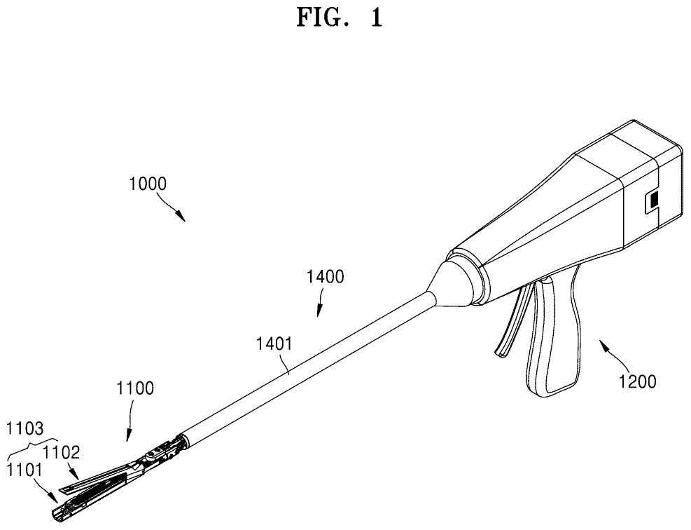

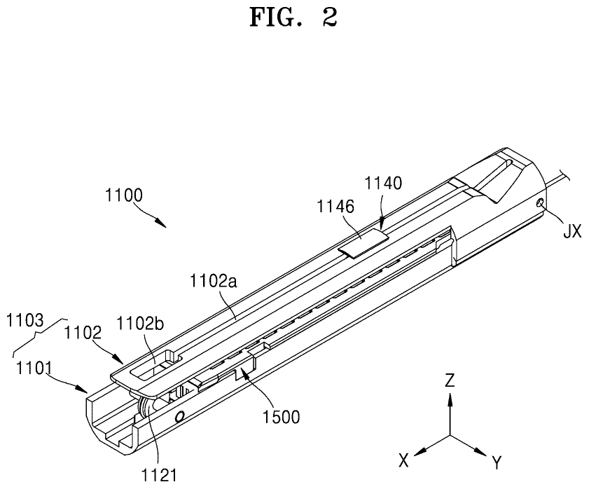

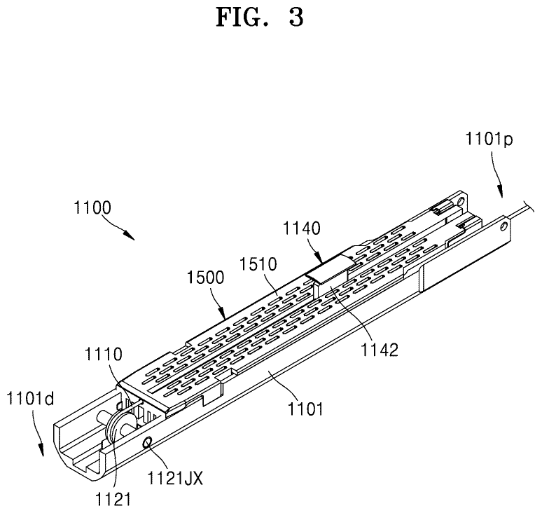

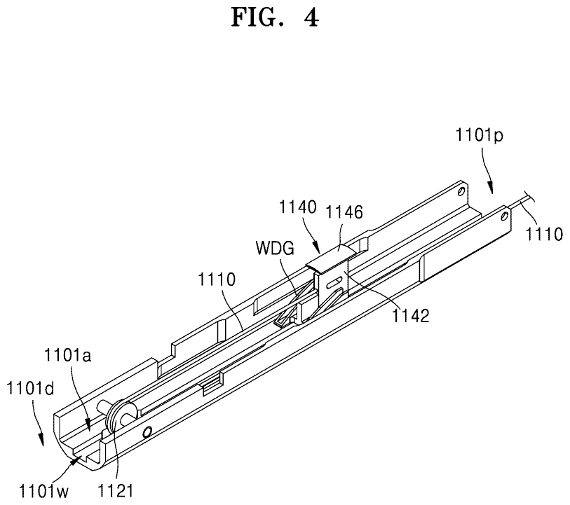

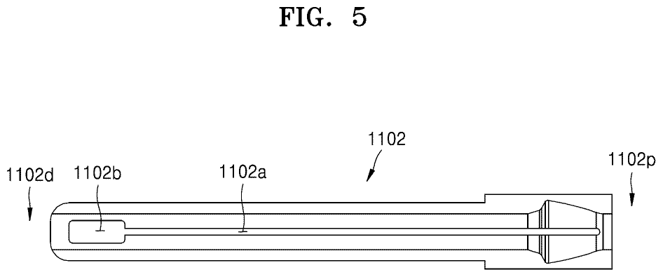

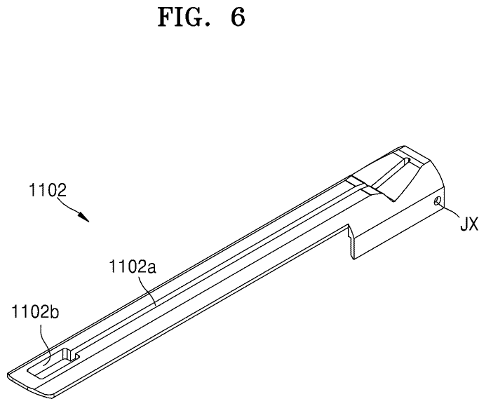

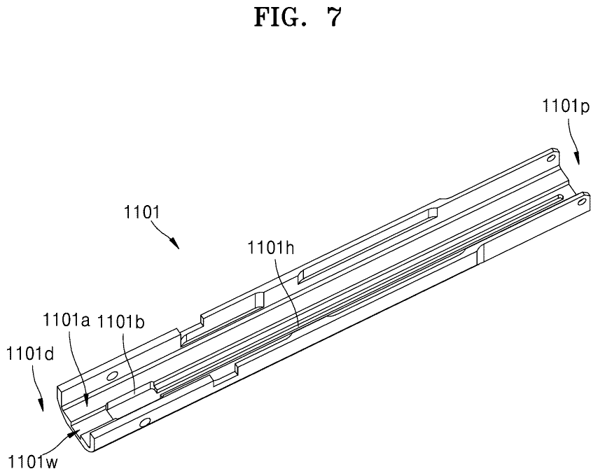

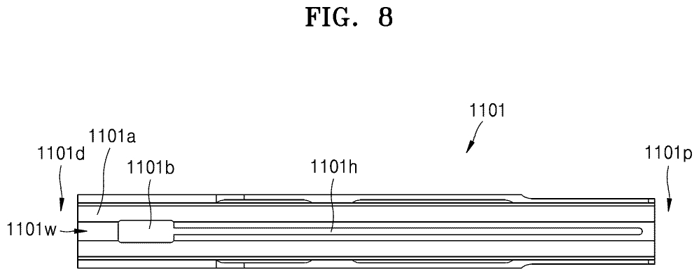

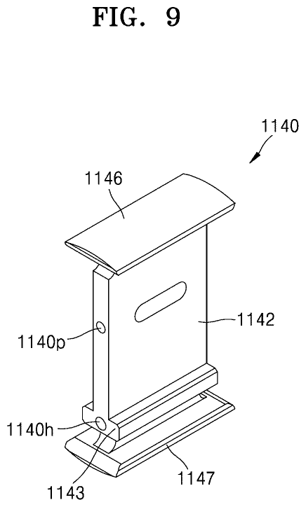

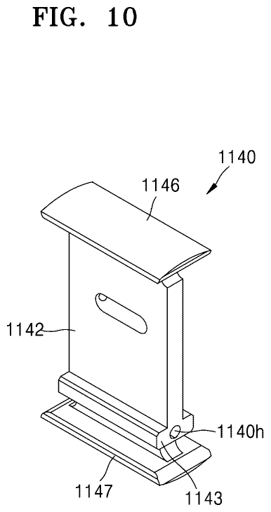

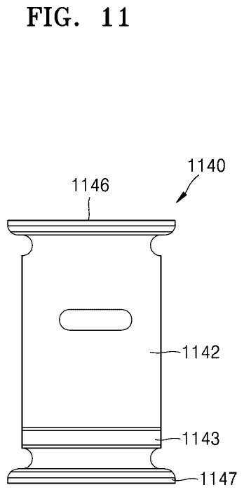

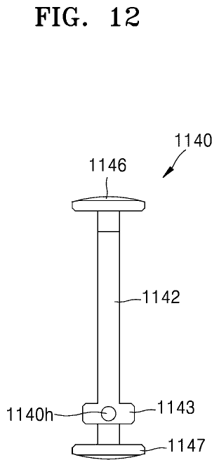

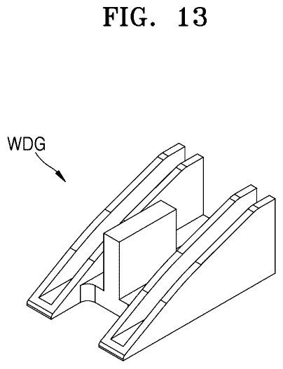

FIG. 1 is a schematic perspective view of a surgical instrument according to an embodiment of the present disclosure. FIG. 2 is a schematic perspective view for describing an end tool of FIG. 1. FIG. 3 is a schematic perspective view of the end tool of FIG. 2 with a second jaw removed. FIG. 4 is a schematic perspective view of the end tool of FIG. 3 with a cartridge removed. FIG. 5 is a plan view schematically illustrating the second jaw of the end tool of FIG. 2. FIG. 6 is a perspective view schematically illustrating the second jaw of the end tool of FIG. 2. FIG. 7 is a perspective view schematically illustrating a first jaw of the end tool of FIG. 2. FIG. 8 is a plan view schematically illustrating the first jaw of the end tool of FIG. 2. FIG. 9 is a perspective view illustrating an operation member of the end tool of FIG. 2. FIG. 10 is a perspective view of the operation member of FIG. 9 viewed from another direction. FIG. 11 is a side view of the operation member of FIG. 9 viewed in one direction. FIG. 12 is a front view of the operation member of FIG. 9 viewed in one direction. FIG. 13 is a perspective view illustrating a wedge of the end tool of FIG. 2.

A surgical instrument 1000 according to the present embodiment may include an end tool 1100, an operator 1200, and a connector 1400.

Here, the connector 1400 is formed in the shape of a hollow shaft, and one or more wires and electric wires may be accommodated therein. The operator 1200 is coupled to one end portion of the connector 1400, the end tool 1100 is coupled to another end portion thereof, and the connector 1400 may serve to connect the operator 1200 to the end tool 1100. As an example, the connector 1400 may include a straight part 1401, and although not shown in the drawings, the connector 1400 may include one or more curved parts to increase ease of use and control the arrangement of components for manipulation.

The operator 1200 is formed at the one end portion of the connector 1400 and provided as an interface to be directly controlled by a medical doctor, and may have, for example, a tongs shape, a stick shape, a lever shape, or the like, and when the medical doctor controls the operator 1200, the end tool 1100, which is connected to the interface and inserted into the body of a surgical patient, performs a certain motion, thereby performing surgery. Here, the operator 1200 is illustrated in FIG. 1 as being formed in a handle shape allowing user's fingers to come into close contact therewith and perform one or more motions, such as pulling or pushing, but the concept of the present disclosure is not limited thereto, and various types of manipulation parts that can be connected to the end tool 1100 and manipulate the end tool 1100 may be possible.

The end tool 1100 is formed on another end portion of the connector 1400, and performs necessary motions for surgery by being inserted into a surgical site. As an example of the end tool 1100, a pair of jaws 1103 for performing a grip motion may be used. However, the concept of the present disclosure is not limited thereto, and various devices for performing surgery may be used as the end tool 1100. For example, a configuration of a cantilever cautery may also be used as the end tool. The end tool 1100 is connected to the operator 1200 by an operating force transmitter (not shown, e.g., a wire or the like), and receives a driving force of the operator 1200 through the operating force transmitter to perform a motion necessary for surgery, such as gripping, cutting, suturing, or the like.

Hereinafter, the end tool 1100 of the surgical instrument 1000 of FIG. 2 will be described in more detail.

FIG. 2 is a schematic perspective view for describing the end tool of FIG. 1. FIG. 3 is a schematic perspective view of the end tool of FIG. 2 with a second jaw removed. FIG. 4 is a schematic perspective view of the end tool of FIG. 3 with a cartridge removed.

The end tool 1100 may include the jaw 1103, a fixed pulley 1121, and a forward-moving wire 1110.

The jaw 1103 may perform various functions, for example, a grip motion, and may include a pair of jaws, e.g., a first jaw 1101 and a second jaw 1102 as a specific example. Here, each of the first jaw 1101 and the second jaw 1102, or a component encompassing the first jaw 1101 and the second jaw 1102 may be referred to as the jaw 1103.

The first jaw 1101 and the second jaw 1102 may be disposed to face each other, may move closer to or move away from each other, and may be formed to rotationally move around, for example, one shaft JX.

A cartridge 1500 may be disposed to be accommodated in the first jaw 1101, and a plurality of staples are disposed inside the cartridge 1500. When an operation member 1140 receives a force through the forward-moving wire 1110 while the first jaw 1101 and the second jaw 1102 are close to each other, such as when the first jaw 1101 and the second jaw 1102 are closed with body tissue interposed therebetween, the operation member 1140 may push and raise the staples while moving toward a distal end 1101d of the first jaw 1101, so that stapling may be performed. At this point, one or more clamps 1146 of the operation member 1140 may protrude to the outside of the first jaw 1101 or the second jaw 1102, allowing the operation member 1140 to move forward while applying pressure to an outer surface of the first jaw 1101 or the second jaw 1102 (an upper surface of the second jaw based on FIG. 2), which facilitates the smooth progression of a stapling process.

Meanwhile, the operation member 1140 may be used together with a wedge WDG. For example, the wedge WDG may be prepared separately from the operation member 1140 and then disposed adjacent to the operation member 1140 in the first jaw 1101. In addition, as another example, the operation member 1140 and the wedge WDG may be integrally formed. The wedge WDG may be disposed on at least one side of a body 1142 and may be formed to have a predetermined inclined surface. That is, the wedge WDG may be formed to be inclined by a certain degree with respect to an extension direction of the end tool 1100. In other words, the wedge WDG may be formed to have a greater height at a proximal end 1101p side of the first jaw 1101 than a distal end 1101d side of the first jaw 1101.

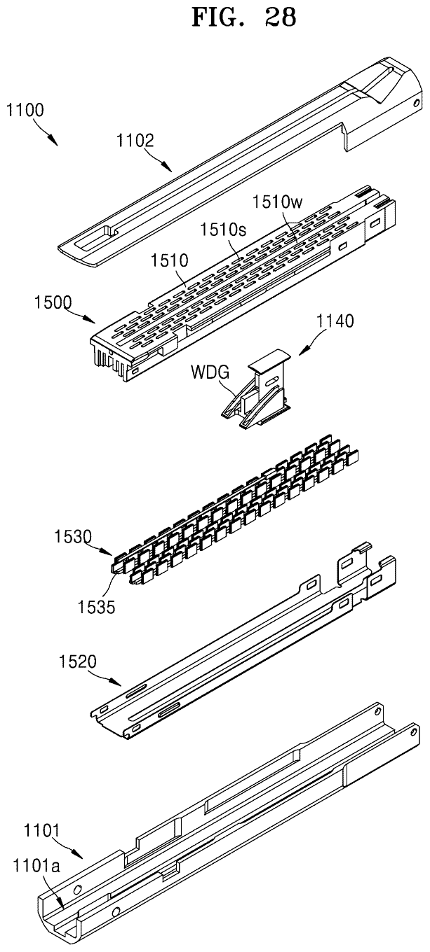

The wedge WDG may be formed to be sequentially in contact with withdrawal members 1535 (refer to FIG. 28) or a plurality of staples 1530 (refer to FIG. 28) disposed in the cartridge 1500, and may serve to sequentially push and raise the staples 1530. More detailed description will be provided below.

FIG. 5 is a plan view schematically illustrating the second jaw of the end tool of FIG. 2. FIG. 6 is a perspective view schematically illustrating the second jaw of the end tool of FIG. 2.

The second jaw 1102 may be formed in an elongated bar shape as a whole, and for example, the second jaw 1102 may be formed in a rod shape to correspond to the first jaw 1101 in at least one region. An anvil is formed at a distal end 1102d side of the second jaw 1102, and a region coupled to the first jaw 1101 may be included in a proximal end 1102p of the second jaw 1102. As an example, the second jaw 1102 may be formed to be rotatable around one shaft JX of the proximal end 1102p with respect to the first jaw 1101.

As a specific example, among surfaces of the second jaw 1102, the surface facing the first jaw 1101 may have the anvil formed thereon, may be formed in a flat plane shape, and may have shapes corresponding to the shapes of the staples 1530 to be described below. The anvil of the second jaw 1102 may serve as a support for supporting the staple 1130 on the opposite side of the operation member 1140 when the operation member 1140 pushes and raises the staple 1130 during a stapling motion, so that the staple 1130 is bent.

The second jaw 1102 includes a guide groove 1102a. The guide groove 1102a may have a shape elongated in a longitudinal direction of the second jaw 1102.

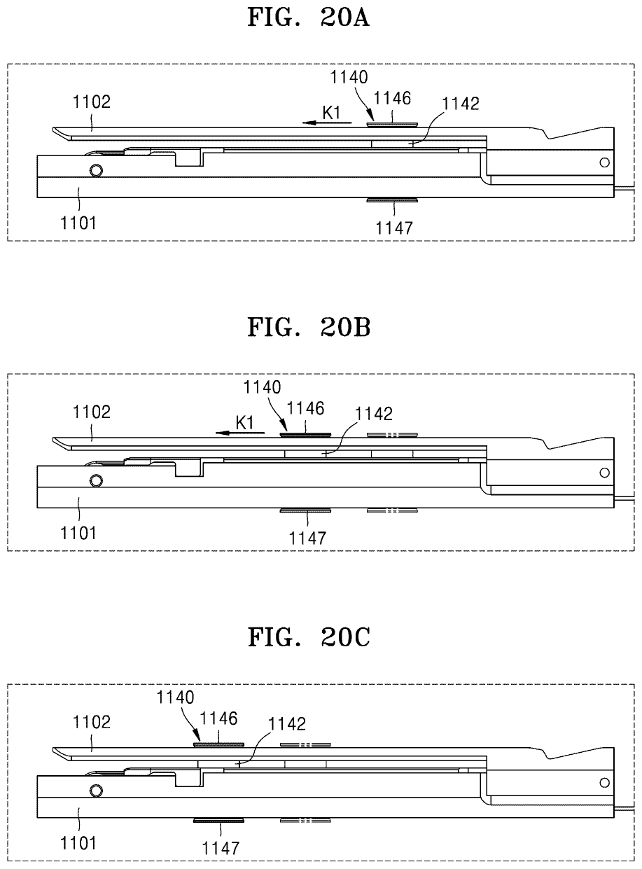

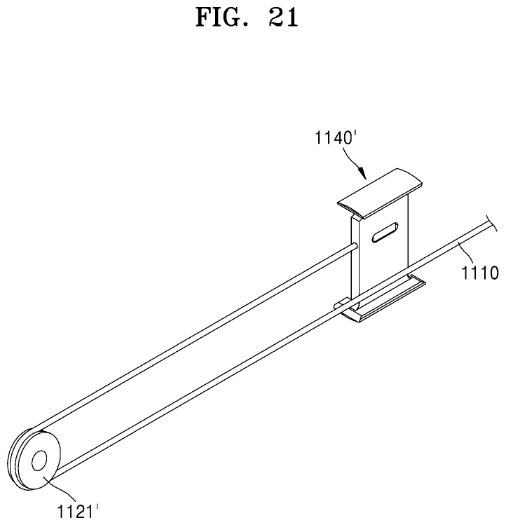

The guide groove 1102a may be formed to guide the operation member 1140, and may be a groove formed to pass through a region facing the operation member 1140. Through this, one region of the operation member 1140, such as at least one region of a body 1142 of the operation member 1140, or a first clamp 1146 connected thereto may pass through the guide groove 1102a to exit to the outside of the second jaw 1102. When the operation member 1140 moves forward, the first clamp 1146 may pass through the guide groove 1102a of the second jaw 1102 to be exposed to the outside of the second jaw 1102, and may come into contact with an upper surface of the second jaw 1102 or apply pressure thereto. As the operation member 1140 moves, the first clamp 1146 applies pressure on the upper surface of the second jaw 1102 and a second clamp 1147 to be described below applies pressure on a lower surface of the first jaw 1101 such that a gap between the second jaw 1102 and the first jaw 1101 decreases, allowing the second jaw 1102 to naturally remain in a closed state with respect to the first jaw 1101 (refer to FIGS. 20A, 20B, and 20C).

In an optional embodiment, the second jaw 1102 may include a window 1102b. After operating the operation member 1140 or using the end tool 1100, the first clamp 1146 of the operation member 1140 may be located corresponding to the window 1102b, and the coupled state of the second jaw 1102 and the operation member 1140 may be released.

FIG. 7 is a perspective view schematically illustrating the first jaw of the end tool of FIG. 2. FIG. 8 is a plan view schematically illustrating the first jaw of the end tool of FIG. 2.

Referring to FIGS. 2 to 4, 7, 8, and the like, the first jaw 1101 may be formed in an elongated bar shape as a whole, and the cartridge 1500 (refer to FIG. 3) may be accommodated in the first jaw 1101 at the distal end 1101d side. A rotation shaft may be disposed in the proximal end so that the first jaw 1101 is rotationally movable therearound, and the rotation shaft may correspond to the rotation shaft JX formed in the second jaw 1102 described above.

The first jaw 1101 may entirely be formed in the form of a hollow box with one surface (upper surface) thereof is removed, such that a cartridge accommodation part 1101a capable of accommodating the cartridge 1500 may be formed inside the first jaw 1101. That is, the first jaw 1101 may be formed in a substantially “U” shape in cross section.

A guide groove 1101h may be formed in a bottom surface of the first jaw 1101, the bottom surface opposite to an upper open region formed by removing one surface. Specifically, the guide groove 1101h may be formed to guide a linear motion of the operation member 1140.

The guide groove 1101h may be formed to guide the operation member 1140, and may be a groove formed to pass through a region facing the operation member 1140. Through this, one region of the operation member 1140, such as at least one region of the body 1142 of the operation member 1140, or the second clamp 1147 connected thereto may pass through the guide groove 1101h to exit to the outside of the first jaw 1101. When the operation member 1140 moves forward, the second clamp 1147 may pass through the guide groove 1101h of the first jaw 1101 to be exposed to the outside of the first jaw 1101, and may come into contact with the lower surface of the first jaw 1101 or apply pressure thereto. As the operation member 1140 moves, the second clamp 1147 applies pressure on the lower surface of the first jaw 1101 and the first clamp 1146 applies pressure on the upper surface of the second jaw 1102 such that a gap between the second jaw 1102 and the first jaw 1101 decreases, allowing the second jaw 1102 to naturally remain in a closed state with respect to the first jaw 1101 (refer to FIGS. 20A, 20B, and 20C).

In an optional embodiment, the first jaw 1101 may include a window 1101b. After operating the operation member 1140 or using the end tool 1100, the second clamp 1147 of the operation member 1140 may be located corresponding to the window 1101b, and the coupled state of the first jaw 1101 and the operation member 1140 may be released.

The first jaw 1101 may include an extension groove 1101w in the bottom surface thereof. The extension groove 1101w may be formed in the bottom surface of the first jaw 1101, i.e., in the bottom surface on which the cartridge accommodation part 1101a accommodating the cartridge therein is formed.

The extension groove 1101w may be formed to overlap a movement path of the operation member 1140, and may have a predetermined length in the longitudinal direction of the first jaw 1101. For example, the extension groove 1101w may be formed to have a length equal to a length of the first jaw 1101.

In an optional embodiment, the extension groove 1101w may be formed to overlap the guide groove 1101h. For example, the extension groove 1101w may be formed in such a shape in which one region of the bottom surface of the first jaw 1101 in which the cartridge accommodation part 1101a is formed is recessed, and the guide groove 1101h overlapping the extension groove 1101w and having a width less than or equal to a width of the extension groove 1101w may be formed to pass through the bottom surface of the first jaw 1101.

Accordingly, the arrangement and motion of the forward-moving wire 1110 and the arrangement and motion of the operation member 1140 may be efficiently controlled.

The operation member 1140 will be described in detail.

FIG. 9 is a perspective view illustrating the operation member of the end tool of FIG. 2. FIG. 10 is a perspective view of the operation member of FIG. 9 viewed from another direction. FIG. 11 is a side view of the operation member of FIG. 9 viewed in one direction. FIG. 12 is a front view of the operation member of FIG. 9 viewed in one direction. FIG. 13 is a perspective view illustrating the wedge of the end tool of FIG. 2.

The operation member 1140 may include the body 1142, the first clamp 1146, and the second clamp 1147. Meanwhile, the operation member 1140 may be used together with the wedge WDG. For example, the wedge WDG may be prepared separately from the operation member 1140 and then disposed adjacent to the operation member 1140 in the first jaw 1101. In addition, as another example, the operation member 1140 and the wedge WDG may be integrally formed. In the present specification, for convenience of description, the operation member 1140 and the wedge WDG will be described and illustrated in the drawings with the assumption that the operation member 1140 and the wedge WDG are prepared separately.

The wedge WDG may be disposed on at least one side of the body 1142 and may be formed to have a predetermined inclined surface. That is, the wedge WDG may be formed to be inclined by a certain degree with respect to the extension direction of the end tool 1100. In other words, the wedge WDG may be formed to have a greater height at the proximal end 1101p side of the first jaw 1101 than the distal end 1101d side of the first jaw 1101.

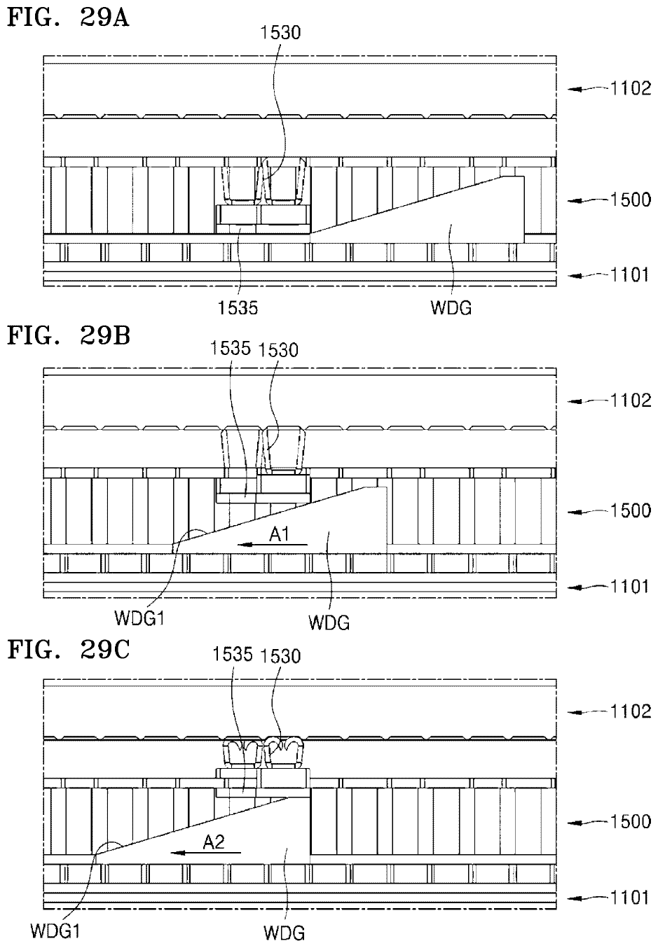

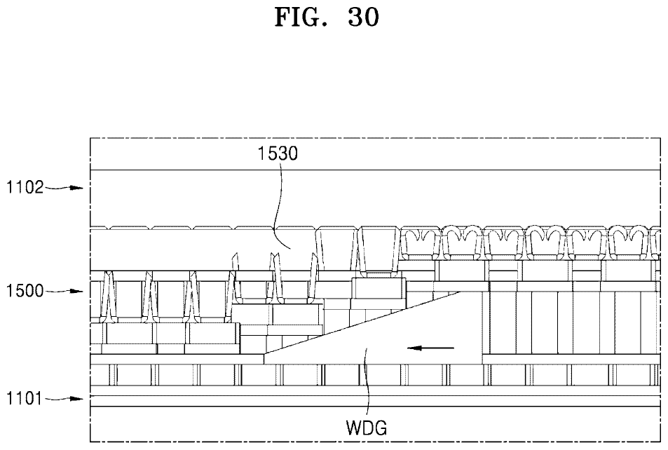

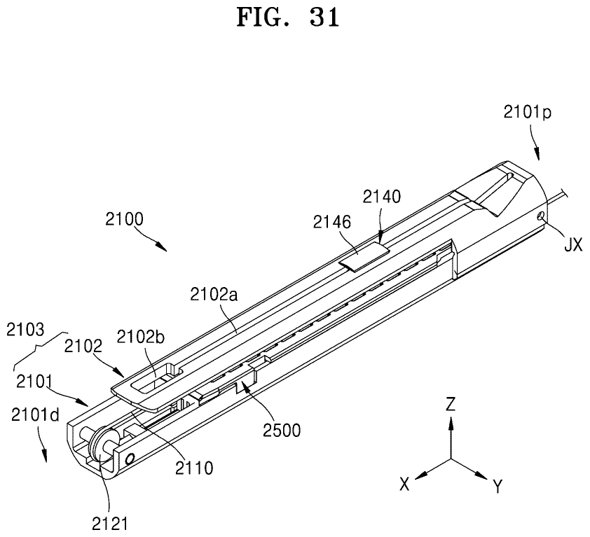

The wedge WDG may be formed to be sequentially in contact with the withdrawal members 1535 (refer to FIG. 28) or the plurality of staples 1530 (refer to FIG. 28) and may serve to sequentially push and raise the staples 1530. As shown in FIGS. 29A, 29B, and 29C and 30 to be described later and elsewhere herein, the operation member 1140 may serve to withdraw the staples 1530 to the outside of the cartridge 1500 by sequentially pushing and raising the staples 1530 while moving toward the distal end 1101d.

The body 1142 may be in the form of an elongated column, such as a plate-shaped column. In addition, a blade region may be formed in one region of the body 1142, and an edge sharply formed to cut tissue may be formed in the blade region. The tissue disposed between the first jaw 1101 and the second jaw 1102 may be cut as at least a portion of the edge formed in one region of the body 1142 is withdrawn to the outside of the first jaw 1101 and the cartridge 1500.

The first clamp 1146 may be formed in one region of the body 1142, and the second clamp 1147 may be formed in another region different from the one region. For example, the body 1142 may be disposed between the first clamp 1146 and the second clamp 1147.

The first clamp 1146 and the second clamp 1147 may be formed to have a width at least greater than that of the body 1142. Accordingly, the first clamp 1146 may be inserted into and pass through the guide groove 1102a formed in the second jaw 1102 in the longitudinal direction to be disposed or brought into contact with the upper surface of the second jaw 1102 and, at the same time, the second clamp 1147 may be inserted into and pass through the guide groove 1101h formed in the first jaw 1101 in the longitudinal direction to be disposed or brought into contact with the lower surface of the first jaw 1101, so that the first clamp 1146 and the second clamp 1147 may move. Thus, when the operation member 1140 moves, the first clamp 1146 and the second clamp 1147 may apply forces in directions that bring the second jaw 1102 and the first jaw 1101 closer to each other.

As a result, when the operation member 1140 moves from the proximal end 1101p of the first jaw 1101 toward the distal end 1101d of the first jaw 1101, a motion of decreasing a distance between the second jaw 1102 and the first jaw 1101, i.e., a closing motion of the jaw 1103, may be naturally implemented through the first clamp 1146 and the second clamp 1147.

A connection region 1140p may be formed in one region of the body 1142, such as one region in a front side of the body 1142, specifically, one region of the body 1142 facing the distal end 1101d of the first jaw 1101.

The connection region 1140p may be a region to which the forward-moving wire 1110 is connected, and for example, may have a fixing groove shape such that one region of an end portion of the forward-moving wire 1110 is accommodated or fixed thereto. When the forward-moving wire 1110 is connected to the connection region 1140p and the forward-moving wire 1110 is pulled, a force for pulling the forward-moving wire 1110 is transmitted to the operation member 1140 through the connection region 1140p, allowing the operation member 1140 to move, i.e., move forward.

A wire path 1140h may be formed to pass through one region of the body 1142, e.g., the front and rear sides of the body 1142. The wire path 1140h may be formed to have a greater width than the forward-moving wire 1110 so that at least one region of the forward-moving wire 1110 passes therethrough and moves.

The wire path 1140h may be formed to be spaced apart from the connection region 1140p.

For example, the wire path 1140h may be disposed below the connection region 1140p, and as a specific example, the wire path 1140h may be disposed closer to the bottom of the operation member 1140, such as the second clamp 1147, than the connection region 1140p.

Accordingly, a region (e.g., the connection region 1140p) of the forward-moving wire 1110 which directly pulls the operation member 1140, and a region (e.g., a region passing through the wire path 1140h) of the forward-moving wire 1110 which moves in the opposite direction to the pulling force may be efficiently disposed, thereby efficiently transmitting a force to the operation member 1140.

In an optional embodiment, the operation member 1140 may include a side part 1143.

The side part 1143 is formed in one region of the body 1142 or a region connected to the body 1142, and is a region corresponding to the wire path 1140h, and may have a greater width than at least the body 1142 and have a shape protruding from both sides of the body 1142 in a width direction as a specific example. By forming the side part 1143 to be wider than the body 1142, a decrease in rigidity of the operation member 1140 due to the width of the operation member 1140 being reduced by the wire path 1140h may be effectively prevented. Accordingly, the operation member 1140 may be improved in durability when moving using the forward-moving wire 1110.

The forward-moving wire 1110 and the fixed pulley 1121 for driving the operation member 1140 will now be described.

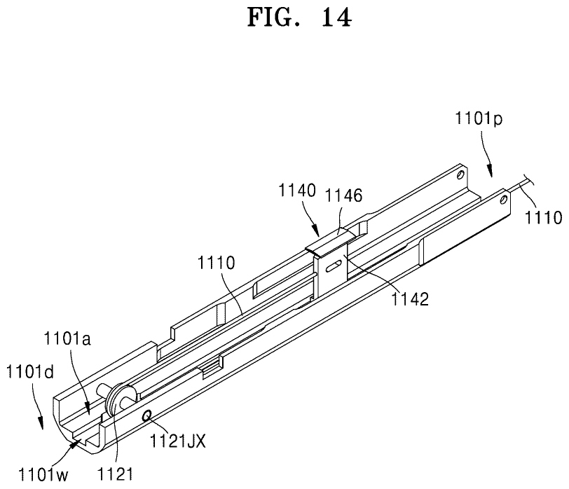

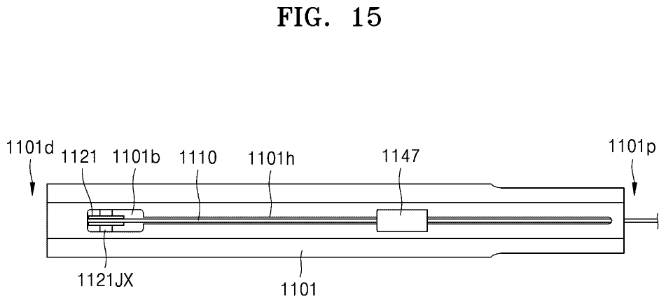

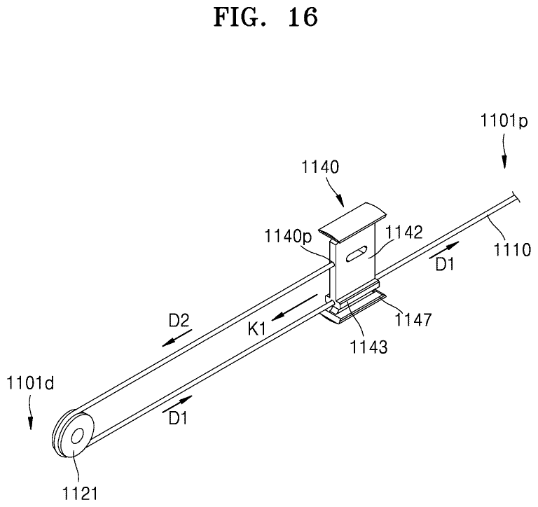

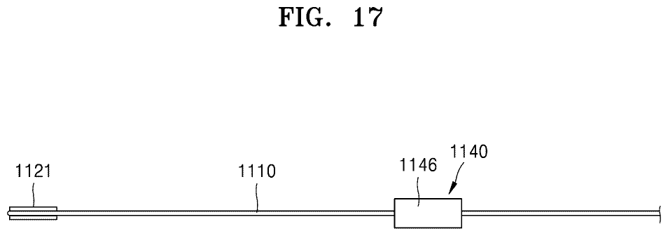

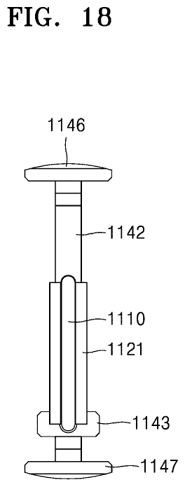

FIG. 14 is a schematic perspective view for describing the operation member, the fixed pulley, and the forward-moving wire of the end tool of FIG. 2. FIG. 15 is a bottom view of FIG. 14 viewed in one direction. FIG. 16 is a perspective view illustrating a state in which the first jaw is removed from FIG. 14. FIG. 17 is a plan view of FIG. 16 viewed in one direction. FIG. 18 is a front view of FIG. 16 viewed in one direction.

The fixed pulley 1121 and the forward-moving wire 1110 may be disposed to move the operation member 1140.

The fixed pulley 1121 may be disposed in the first jaw 1101, for example, may be connected to a pulley shaft 1121JX formed in a direction transverse to the width direction of the first jaw 1101, and may be coupled to or integrally formed with the pulley shaft 1121JX. In an optional embodiment, the fixed pulley 1121 may be formed distinct from the pulley shaft 1121JX so as to rotationally move around the pulley shaft 1121JX.

The forward-moving wire 1110 may be wound around an outer circumferential surface of the fixed pulley 1121, and to this end, a groove may be formed on the outer circumferential surface of the fixed pulley 1121.

The fixed pulley 1121 may be disposed closer to the distal end 1101d of the first jaw 1101 at least than the operation member 1140.

The fixed pulley 1121 may have a vertically disposed shape, for example, may be disposed parallel to a direction from the first jaw 1101 to the second jaw 1102, and may be disposed to correspond to a center line of the first jaw 1101.

At least one region, such as a lower region, of the fixed pulley 1121 may be disposed to correspond to the extension groove 1101w of the first jaw 1101.

Since the forward-moving wire 1110 is correspondingly wound around the fixed pulley 1121, one region of the forward-moving wire 1110 may also be disposed to correspond to the extension groove 1101w of the first jaw 1101.

Accordingly, the forward-moving wire 1110 and the fixed pulley 1121 may be efficiently disposed while easily securing a space for moving the operation member 1140 of the first jaw 1101 and stapling.

The forward-moving wire 1110 may extend to have a length in the longitudinal direction of the first jaw 1101. In addition, one end portion region of the forward-moving wire 1110 may extend to the proximal end 1101p of the first jaw 1101 to be connected to the inside of a driving part, such as the operator 1200 (refer to FIG. 1), and pulled through the manipulation of the operator 1200. Another end portion of the forward-moving wire 1110 extends in a direction toward the distal end 1101d of the first jaw 1101 in the longitudinal direction of the first jaw 1101, passes through the wire path 1140h of the operation member 1140, emerges from an upper side of the fixed pulley 1121 after being wound around a lower side of the fixed pulley 1121, and then extends in a direction toward the proximal end 1101p of the first jaw 1101 to be connected and fixed to the connection region 1140p of the operation member 1140.

In an optional embodiment, the region of the forward-moving wire 1110, which extends toward the distal end 1101d of the first jaw 1101, passes through the wire path 1140h of the operation member 1140, and then directs toward the lower side of the fixed pulley 1121, may be parallel to the region of the forward-moving wire 1110, which emerges from the upper side of the fixed pulley 1121, extends toward the proximal end 1101p of the first jaw 1101, and directs toward the connection region 1140p of the operation member 1140.

Accordingly, when pulling the forward-moving wire 1110 toward the proximal end 1101p, a pulling force may be effectively transmitted to the operation member 1140, and the distance the forward-moving wire 1110 is pulled and the distance the operation member 1140 moves forward may be controlled to be approximately similar or equal to each other. In addition, as an example, when pulling the forward-moving wire 1110 toward the proximal end 1101p, a ratio of the pulling force to the corresponding forward-moving distance of the operation member 1140 may be precisely controlled at a predetermined ratio. As a specific example, when pulling the forward-moving wire 1110 toward the proximal end 1101p, the ratio of the pulling force to the forward-moving distance of the operation member 1140 may be easily controlled to a value equal to, or substantially equal or similar to 1:1.

Accordingly, as shown in FIG. 16, when the forward-moving wire 1110 is pulled in a first direction D1, one region of the forward-moving wire 1110 passes through the wire path 1140h of the operation member 1140 and moves in the first direction D1, so that, the region of the forward-moving wire 1110, which emerges from the upper side of the fixed pulley 1121 after being wound around the lower side of the fixed pulley 1121, moves in a second direction D2, which is the opposite direction of the first direction D1. Accordingly, a force of the forward-moving wire 1110 is transmitted to the connection region 1140p connected to the forward-moving wire 1110, and the force causes the operation member 1140 to move in a direction K1, which is the same direction as the second direction D2, i.e., to move forward.

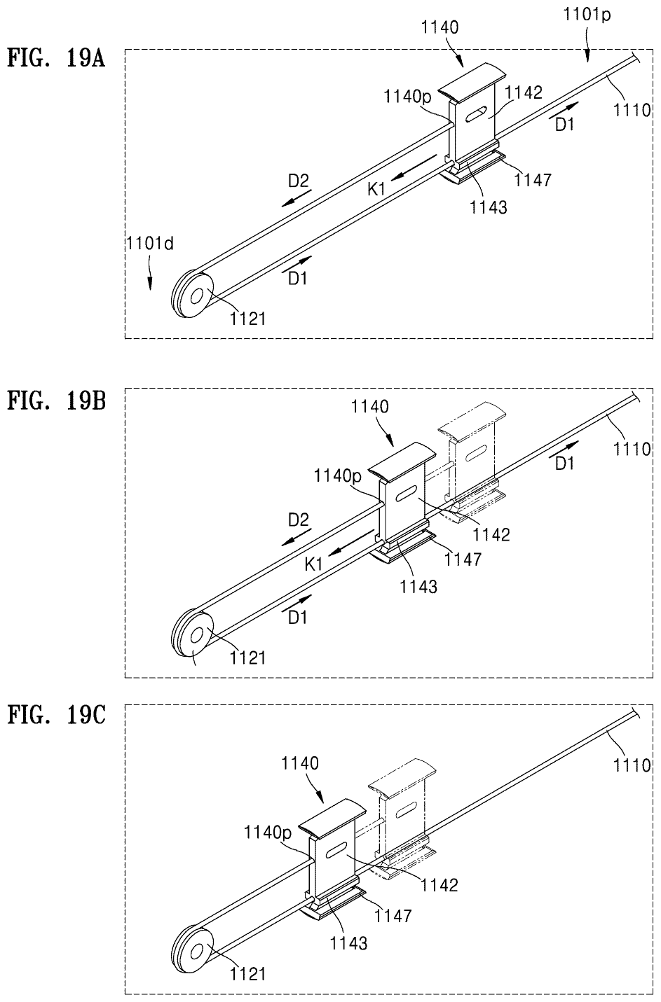

FIGS. 19A, 19B, and 19C are schematic perspective views for describing an operation of the operation member of the end tool of FIG. 2.

Referring to FIG. 19A, the first jaw 1101 is excluded for convenience of description, and the forward-moving wire 1110, the fixed pulley 1121, and the operation member 1140 are illustrated.

As shown in FIG. 19A, when the forward-moving wire 1110 is pulled in the first direction D1, one region of the forward-moving wire 1110 passes through the wire path 1140h of the operation member 1140 and is pulled in the first direction D1, so that, the region of the forward-moving wire 1110, which emerges from the upper side of the fixed pulley 1121 after being wound around the lower side of the fixed pulley 1121, moves in the second direction D2, which is the opposite direction of the first direction D1. Accordingly, a force of the forward-moving wire 1110 is transmitted to the connection region 1140p connected to the forward-moving wire 1110, and the force causes the operation member 1140 to move in a direction K1, which is the same direction as the second direction D2, i.e., to move forward, thereby positioning the operation member 1140 in an advanced position shown in FIG. 19B.

Thereafter, when the forward-moving wire 1110 is further pulled in the first direction D1 as illustrated in FIG. 19B, one region of the forward-moving wire 1110 passes through the wire path 1140h of the operation member 1140 and is pulled further in the first direction D1, so that, the region of the forward-moving wire 1110, which emerges from the upper side of the fixed pulley 1121 after being wound around the lower side of the fixed pulley 1121, moves further in the second direction D2, which is the opposite direction of the first direction D1. Accordingly, a force of the forward-moving wire 1110 is transmitted to the connection region 1140p connected to the forward-moving wire 1110, and the force causes the operation member 1140 to move further in the direction K1, which is the same direction as the second direction D2, i.e., to move forward to a position forward than that in FIG. 19B, and thus, as shown in FIG. 19C, the operation member 1140 moves forward to a further advanced position than that in FIG. 19B.

FIGS. 20A, 20B, and 20C are schematic side views for describing an operation of the operation member of the end tool of FIG. 2. For example, FIGS. 20A, 20B, and 20C may be a side view illustrating the state of FIGS. 19A, 19B, and 19C.

For example, FIGS. 20A, 20B, and 20C may be side views of the end tool 1100 in states corresponding to FIGS. 19A, 19B, and 19C.

Referring to FIG. 20A, the first jaw 1101 and the second jaw 1102 are in a closed state, and the first clamp 1146 of the operation member 1140 may be inserted into the guide groove 1102a (refer to FIG. 5), which is formed in the second jaw 1102 in the longitudinal direction, and disposed on or brought into contact with the upper surface of the second jaw 1102 through the guide groove 1102a, and the second clamp 1147 of the operation member 1140 may be inserted into the guide groove 1101h (refer to FIG. 7), formed in the first jaw 1101 in the longitudinal direction, and disposed on or brought into contact with the lower surface of the first jaw 1101 through the guide groove 1101h (refer to FIG. 7).

In this state, when the forward-moving wire 1110 is pulled in the first direction D1 as shown in FIG. 19A described above, the operation member 1140 is also moved in a forward movement direction K1 of FIG. 20A due to the forward-moving wire 1110, thereby positioning the operation member 1140 in an advanced position shown in FIG. 20B.

Thereafter, when the forward-moving wire 1110 is pulled further in the first direction D1 as shown in FIG. 19B described above, the operation member 1140 is also further moved in the forward movement direction K1 of FIG. 20B due to the forward-moving wire 1110, thereby positioning the operation member 1140 in an advanced position shown in FIG. 20C.

As a result, when the operation member 1140 moves from the proximal end 1101p of the first jaw 1101 toward the distal end 1101d, a force may be applied in a direction in which the second jaw 1102 is closer to the first jaw 1101 through the first clamp 1146 and the second clamp 1147, that is, the closing motion of the jaw 1103 may be naturally implemented, the closed state of the first jaw 1101 and the second jaw 1102 may be stably maintained and precisely controlled during stapling, and motion balance during stapling may be improved.

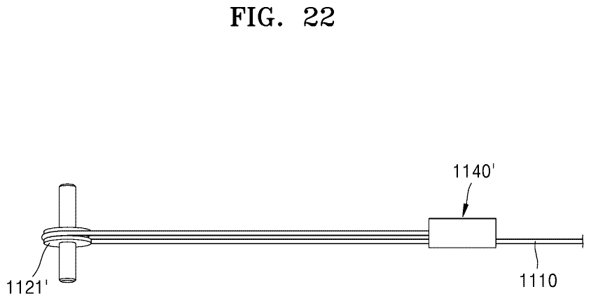

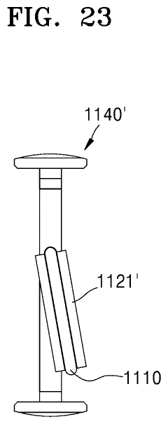

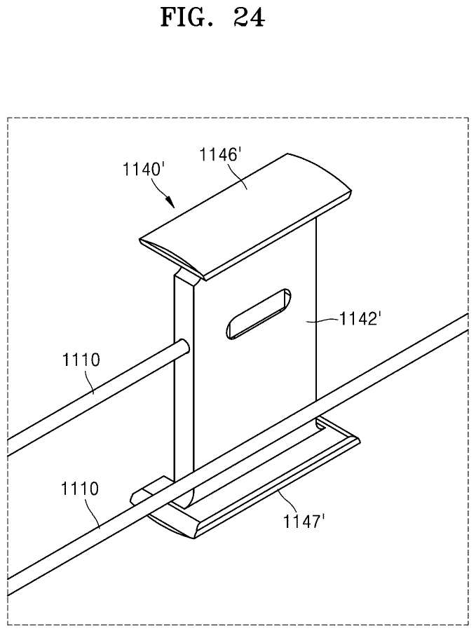

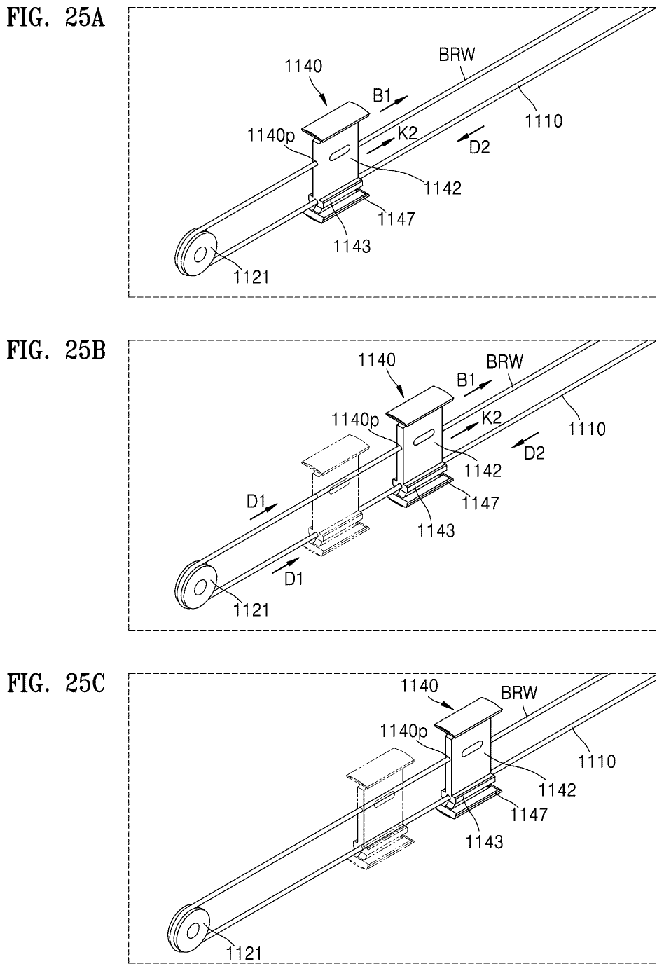

FIGS. 21 to 24 are views for describing one modified example of the end tool of FIG. 2. FIGS. 25A, 25B, and 25C are views for describing an optional embodiment in which a backward-moving wire is added to the end tool of FIG. 2.

Referring to FIGS. 21 to 24, the end tool of the present modified example is different in an operation member 1140′ and a fixed pulley 1121′, which will be mainly described below.

The fixed pulley 1121′ of the present embodiment may be disposed to have an inclined shape rather than a vertical shape. For example, the fixed pulley 1121′ may be disposed to be inclined at a predetermined angle without being parallel to a direction from the first jaw 1101 (refer to FIG. 2) to the second jaw 1102 (refer to FIG. 2). Accordingly, in the path of the forward-moving wire 1110 correspondingly wound around the fixed pulley 1121′, regions where the forward-moving wire 1110 enters and emerges do not overlap nor are parallel to each other in one region.

As a specific example, the region of the forward-moving wire 1110 extending from the proximal end of the first jaw toward the fixed pulley 1121′ may not overlap the operation member 1140′, and accordingly, the operation member 1140′ does not require the wire path 1140h (refer to FIG. 9) of the embodiment described above, and there is no need to form the side part 1143 (refer to FIG. 9) thickened due to the wire path.

Accordingly, manufacturing efficiency and precision manufacturing characteristics of the operation member 1140 are improved, thereby improving durability and manufacturing convenience of the operation member 1140.

FIGS. 25A, 25B, and 25C are views for describing an optional embodiment in which a backward-moving wire is added to the end tool of FIG. 2.

Referring to FIGS. 25A, 25B, and 25C, the end tool of the present embodiment may further include a backward-moving wire BRW.

The backward-moving wire BRW may be connected to one region of the operation member 1140 and may be connected, for example, to a rear side of the operation member 1140, specifically, to a surface of the operation member 1140, which is opposite to the surface on which the connection region 1140p of the body 1142 connected to the forward-moving wire 1110 is formed.

A driving part or driving transmission part (e.g., a wire, a pulley, or the like) capable of pulling the backward-moving wire BRW may be connected to the backward-moving wire BRW, and the backward-moving wire BRW may be operated according to manual or automatic manipulation. For example, the backward-moving wire BRW may be pulled by the operator 1200 (refer to FIG. 1).

By pulling the backward-moving wire BRW. The operation member 1140 may move backward.

For example, as shown in FIG. 25A, the backward-moving wire BRW is pulled in a reverse direction B1 in a state in which the operation member 1140 is located adjacent to the distal end 1101d of the first jaw 1101, the operation member 1140 connected to the backward-moving wire BRW moves backward in a direction K2, which is the same direction as the reverse direction B1. In this case, a force pulling the forward-moving wire 1110 may not be applied.

When the operation member 1140 moves backward (in the direction K2), the region of the forward-moving wire 1110 connected to the connection region 1140p of the operation member 1140 moves in the first direction D1, which is the same direction as the direction K2, and the region of the forward-moving wire 1110, which is wound around the lower side of the fixed pulley 1121 and disposed below the fixed pulley 1121, may move in the second direction D2, which is the opposite direction of the direction K2. Accordingly, the operation member 1140 moves backward to a position as shown in FIG. 25B, having moved backward to be closer to the proximal end 1101p compared to its position in FIG. 25A.

Thereafter, as shown in FIG. 25B, when the backward-moving wire BRW is pulled further in the reverse direction B1, the operation member 1140 connected to the backward-moving wire BRW moves backward in the direction K2, which is the same direction as the reverse direction B1. At this point, the forward-moving wire 1110 may be in a state where no pulling force is applied. When the operation member 1140 moves backward (in the direction K2), the region of the forward-moving wire 1110 connected to the connection region 1140p of the operation member 1140 moves in the first direction D1, which is the same direction as the direction K2), and the region of the forward-moving wire 1110, which is wound around the lower side of the fixed pulley 1121 and disposed below the fixed pulley 1121, may move in the second direction D2, which is the opposite direction of the direction K2. Accordingly, the operation member 1140 moves backward to a position as shown in FIG. 25C, having moved backward to be closer to the proximal end 1101p compared to its position in FIG. 25B.

In an optional embodiment, the forward-moving wire 1110 and the backward-moving wire BRW may be connected to a single common pulley (not shown), such that the forward-moving wire 1110 is pulled by rotating the common pulley in one direction to cause the operation member 1140 to move forward, and the backward-moving wire BRW is pulled by rotating the common pulley in another one direction to cause the operation member 1140 to move backward. In addition, the forward-moving wire 1110 and the backward-moving wire BRW form one loop, and it may be possible to use the adjustment of a loop's movement direction as a means (e.g., an actuator) for moving the operation member forward and backward. Further, it may be easily implemented that the forward-moving wire 1110 and the backward-moving wire BRW move in a ratio of 1:1. Further, by applying pre-tension to the loop, the wire can be controlled so as not to deviate from the path corresponding to the various pulleys for manipulation (e.g., yaw/pitch pulleys and the like).

In addition, when the forward-moving wire 1110 and the backward-moving wire BRW are connected to the single common pulley (not shown), the common pulley may be disposed in various regions according to design convenience, and may be optionally disposed in one region of the end tool 1100, in one region of the connector 1400 or in one region of the operator 1200.

Of course, as another example, separate pulleys for driving the forward-moving wire 1110 and the backward-moving wire BRW may be disposed.

In an optional embodiment, at least one region of the forward-moving wire 1110 and the backward-moving wire BRW may not be both placed in a central plane at least the proximal end 1101p of the end tool 1100. The path of the forward-moving wire 1110 or the backward-moving wire BRW may be controlled through a pulley or the like, such that the forward-moving wire 1110 or the backward-moving wire BRW is not placed in a plane through which the center line of the end tool 1100 passes, which can facilitate the arrangement of the components for yaw and pitch motions of the end tool 1100.

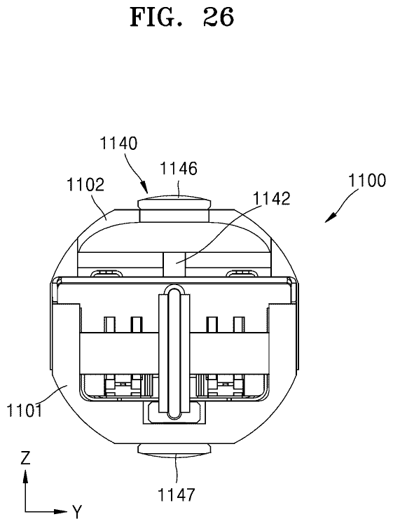

FIG. 26 is a front view of the end tool of FIG. 2 viewed in one direction.

Referring to FIG. 26, as described above, the first clamp 1146 is inserted into the guide groove 1102a (refer to FIG. 5) formed in the second jaw 1102 in the longitudinal direction, and passes through the guide groove 1102a to be disposed on or brought into contact with the upper surface of the second jaw 1102. The second clamp 1147 is inserted into the guide groove 1101h (refer to FIG. 7) formed in the first jaw 1101 in the longitudinal direction, and passes through the guide groove 1101h to be disposed on or brought into contact with the lower surface of the first jaw 1101, allowing the first clamp 1146 and the second clamp 1147 to move. Thus, when the operation member 1140 moves, the first clamp 1146 and the second clamp 1147 may apply forces in directions that bring the second jaw 1102 and the first jaw 1101 closer to each other. At this point, for effective pressure application and durability, in each of the first clamp 1146 and the second clamp 1147, a thickness of a central region may be greater than a thickness of an edge region.

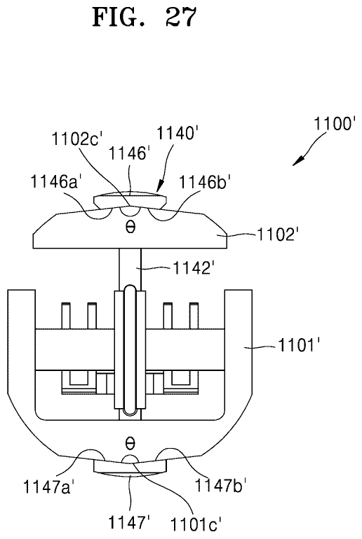

FIG. 27 is a view illustrating another modified example of the end tool of FIG. 2.

Referring to FIG. 27, both sides of an upper surface of a second jaw 1102′ may be bent to have at least a bending angle θ, for example, a protrusion 1102c′ protruding from the upper surface may be formed.

Both sides of a lower surface of a first jaw 1101′ may be bent to have at least a bending angle θ, for example, a protrusion 1101c′ protruding from the lower surface may be formed.

A first clamp 1146′ of an operation member 1140′ is disposed on or brought into contact with the upper surface of the second jaw 1102′. At this point, the first clamp 1146′ may be formed to have a shape that is bent according to the bending shape of the second jaw 1102′. As a specific example, the first clamp 1146′ may have a first side part 1146a′ and a second side part 1146b′ corresponding to the both sides of the protrusion 1102c′, and the first side part 1146a′ and the second side part 1146b′ may be bent on both sides to have the bending angle θ.

A second clamp 1147′ of the operation member 1140′ is disposed on or brought into contact with the lower surface of the first jaw 1101′. At this point, the second clamp 1147′ may be formed to have a shape that is bent according to the bending shape of the first jaw 1101″. As a specific example, the second clamp 1147′ may have a first side part 1147a′ and a second side part 1147b′ corresponding to the both sides of the protrusion 1101c′, and the first side part 1147a′ and the second side part 1147b′ may be bent on both sides to have the bending angle θ.

With such a structure, the first clamp 1146′ and the second clamp 1147′ of the operation member 1140′ may more effectively apply force to the first jaw 1101′ and the second jaw 1102′, which may improve the alignment characteristics of the first jaw 1101′ and the second jaw 1102′ and reduce or prevent misalignment.

For example, due to the centrally bent shape of the first clamp 1146′ and the second clamp 1147′, when the operation member 1140′ move forward, a force may be continuously applied in a direction in which the first jaw 1101′ and the second jaw 1102′ are aligned, and when a repulsive force increases in a direction in which the first jaw 1101′ and the second jaw 1102′ misaligned, the force at the contact regions of the first clamp 1146′ and the second clamp 1147′ with the first jaw 1101′ and the second jaw 1102′ may also increase, thereby increasing a force to keep the first jaw 1101′ and the second jaw 1102′ aligned. Accordingly, during the process of stapling the body tissue, the alignment of the first jaw 1101′ and the second jaw 1102′ can be easily controlled and the durability can be improved, thereby improving the uniformity and accuracy of the stapling.

The cartridge 1500 accommodated in the end tool 1100 of FIG. 2 and a stapling motion will now be described in more detail.

FIG. 28 is a perspective view illustrating the first jaw and the cartridge of the surgical instrument of FIG. 1. FIGS. 29A, 29B, and 29C and 30 are cross-sectional views illustrating a staple motion of the end tool of the surgical instrument of FIG. 1 as a whole.

Referring to FIGS. 1, 2, 3, and 28 and the like, the cartridge 1500 may be disposed in the first jaw 1101, and for example, the cartridge 1500 may be disposed by being coupled to the cartridge accommodation part 1101a of the first jaw 1101. For example, the cartridge 1500 may be integrally formed with the first jaw 1101 while the operation member 1140 is disposed in the first jaw 1101. In addition, in an optional embodiment, the cartridge 1500 may be formed to be mountable to and dismountable from the first jaw 1101.

The cartridge 1500 includes a plurality of staples 1530 therein to perform suturing of tissue, and performs cutting through the operation member 1140. Here, the cartridge 1500 may include a cover 1510, the staples 1530, and the withdrawal members 1535.

The cover 1510 may be formed to cover an upper portion of the cartridge accommodation part 1101a of the first jaw 1101. Staple holes 1510s through which the plurality of staples 1530 may be ejected to the outside may be formed in the cover 1510. As the staples 1530, which are accommodated inside the cartridge accommodation part 1101a before a stapling operation, are pushed and raised upward by the operation member 1140 during a stapling motion, and pass through the staple holes 1510s of the cover 1510 to be withdrawn to the outside of the cartridge 1500, stapling may be performed.

Meanwhile, a slit 1510w may be formed in the cover 1510 along a longitudinal direction of the cover 1510. A blade of the body 1142 of the operation member 1140 may protrude out of the cartridge 1500 through the slit 1510w. As the blade of the body 1142 of the operation member 1140 passes along the slit 1510w, staple-completed tissue may be cut.

In an optional embodiment, the cartridge 1500 may include a case 1520, and the cartridge 1500 may be disposed in the case 1520 after the case 1520 is disposed in the cartridge accommodation part 1101a of the first jaw 1101.

The plurality of staples 1530 may be disposed inside the cartridge accommodation part 1101a of the first jaw 1101. As the operation member 1140 linearly moves in one direction, the plurality of staples 1530 are sequentially pushed and raised from the inside of the cartridge accommodation part 1101a of the first jaw 1101 to the outside, thereby performing suturing, that is, stapling. Here, the staples 1530 may include a material that is durable and does not have an abnormal effect on the human body, such as titanium, stainless steel, or the like.

Meanwhile, the withdrawal members 1535 may be further disposed between the cartridge accommodation part 1101a of the first jaw 1101 and the staples 1530. In other words, it may be said that the staple 1530 is disposed above the withdrawal member 1535. In this case, the operation member 1140 linearly moves in one direction to push and raise the withdrawal member 1535, and the withdrawal member 1535 may push and raise the staple 1530.

As such, the operation member 1140 may be described as pushing and raising the staples 1530 in both the case in which the operation member 1140 directly pushes and raises the staples 1530 and the case in which the operation member 1140 pushes and raises the withdrawal members 1535 and the withdrawal members 1535 pushes and raises the staples 1530 (i.e., the operation member 1140 indirectly pushes and raises the staples 1530).

As described above, the operation member 1140 may be disposed inside the cartridge accommodation part 1101a of the first jaw 1101. In addition, the operation member 1140 may include the wedge WDG or may be used in conjunction with wedge WDG, and when the operation member 1140 moves, the wedge WDG may move together therewith so that the wedge WDG may directly push and raise the staple 1530, or the wedge WDG may push and raise the withdrawal member 1535 to push and raise the staple 1530.

As described above, as the forward-moving wire 1110 moves, i.e., the forward-moving wire 1110 is pulled, the operation member 1140 connected thereto may move forward toward the distal end 1101d of the first jaw 1101.

The forward movement of the operation member 1140 may cause the wedge WDG to push and raise the withdrawal member 1535, which may also cause the staple 1530 to rise, and at the same time, cutting using the blade of the body 1142 of the operation member 1140 may be performed. In addition, in an optional embodiment, in the case of the end tool in which the backward-moving wire BRW is connected to the operation member 1140, the backward-moving wire BRW may be pulled to cause the operation member 1140 to move toward the proximal end 1101p of the first jaw 1101.

Referring to FIGS. 29A, 29B, and 29C and 30, in the state as shown in FIG. 29A, as the operation member 1140 moves in the direction of an arrow A1 of FIG. 29B, the wedge WDG, specifically, an inclined surface WDG1 of the wedge WDG pushes and raises the withdrawal member 1535, and the withdrawal member 1535 pushes and raises one side of a lower portion of the staple 1530. In addition, due thereto, the staple 1530 is ejected to the outside of the first jaw 1101 and the cartridge 1500.

In this state, when the operation member 1140 further moves in the direction of an arrow A2 of FIG. 29C, the ejected staple 1530 is continuously pushed and raised by the operation member 1140 while in contact with the lower surface of the second jaw 1102, such as the anvil, so that stapling is performed while both end portions of the staple 1530 are bent.

As such motions are continuously performed, stapling is sequentially performed from the staple 1530 at the proximal end 1101p side (refer to FIG. 3) to the staple 1530 at the distal end 1101d side (refer to FIG. 3) among the plurality of staples 1530, as shown in FIG. 30.

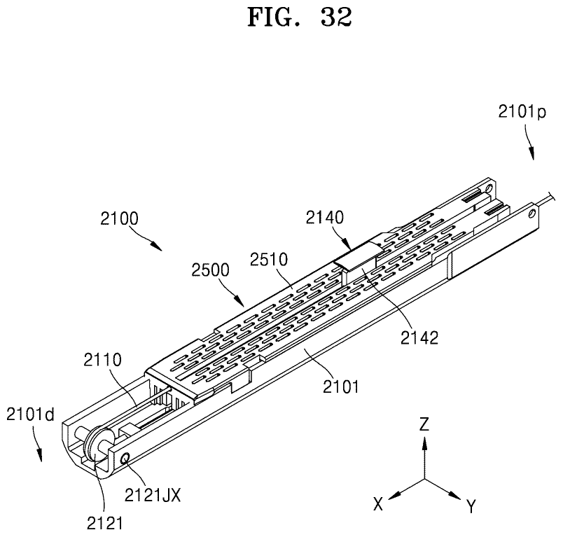

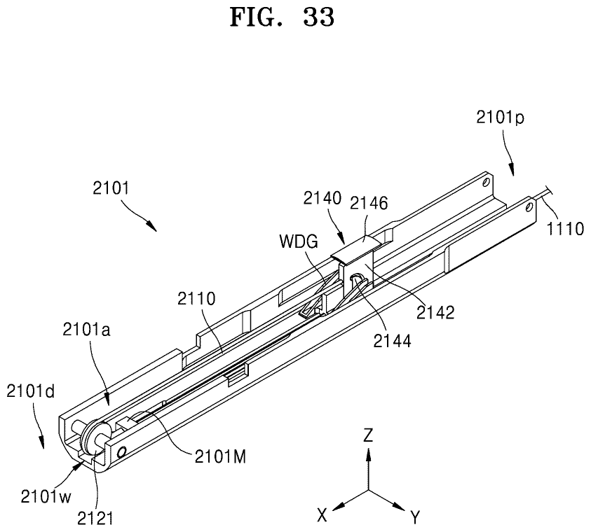

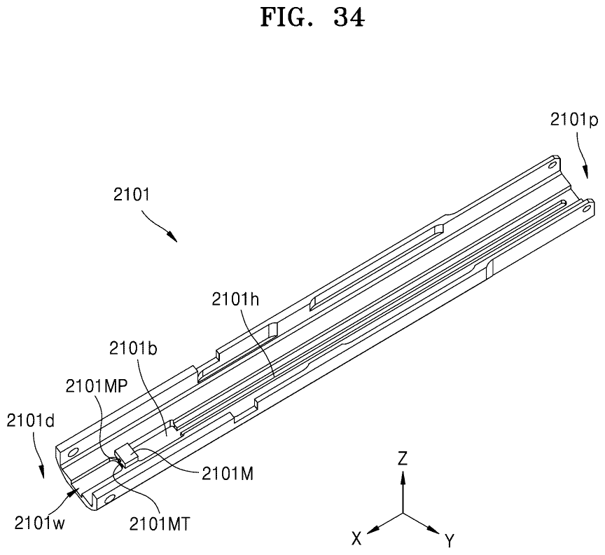

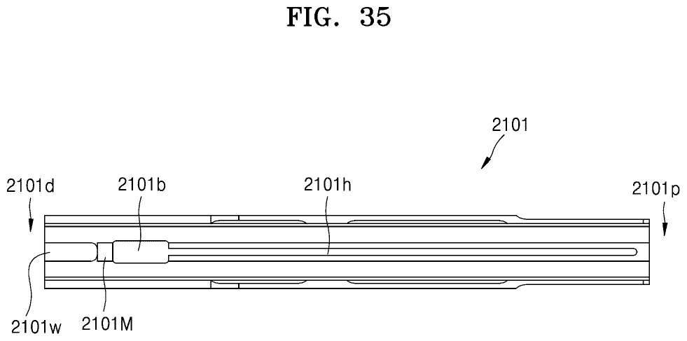

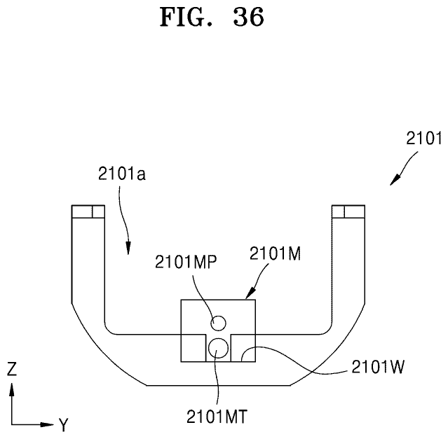

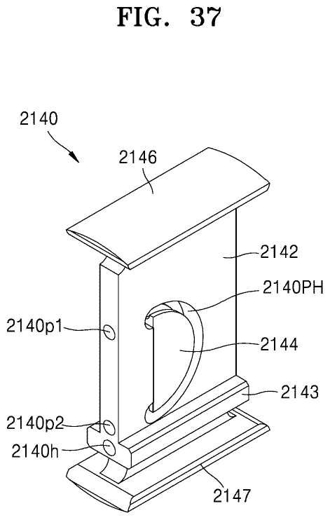

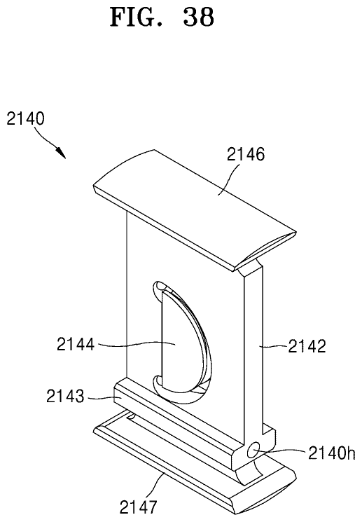

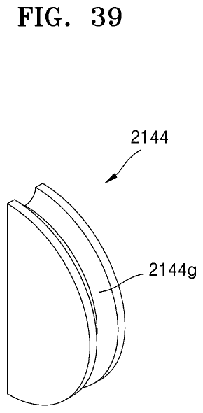

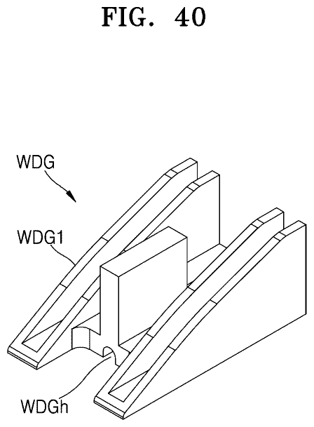

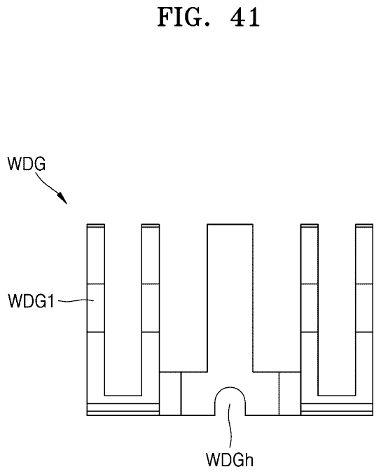

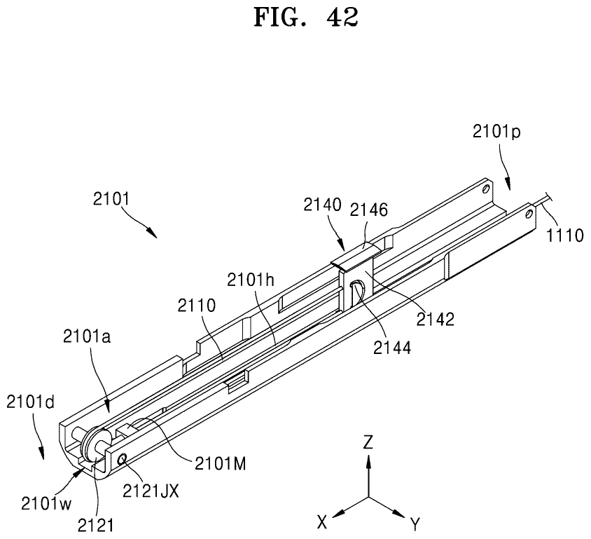

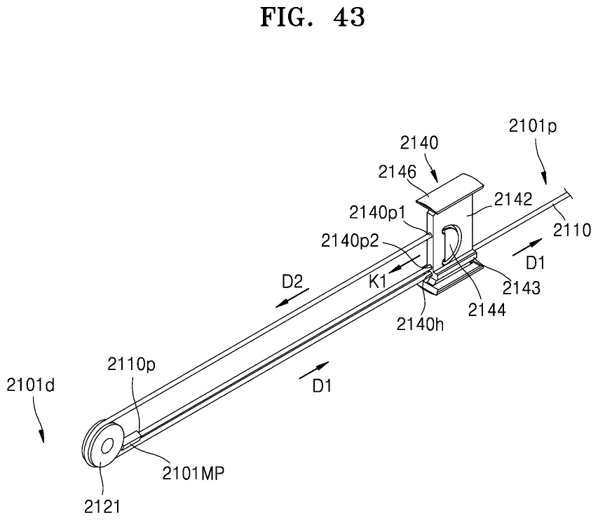

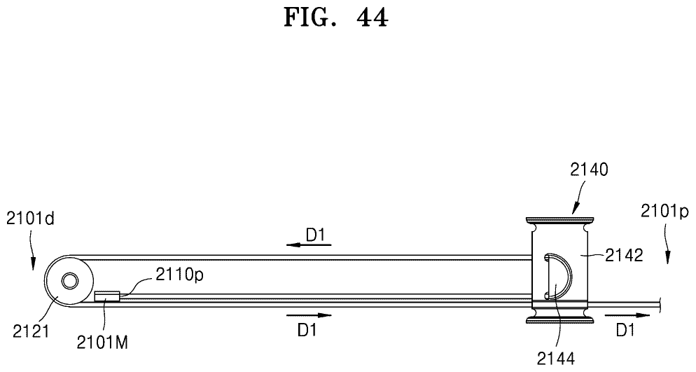

FIG. 31 is a perspective view schematically illustrating an end tool of a surgical instrument according to another embodiment of the present disclosure. FIG. 32 is a schematic perspective view of the end tool of FIG. 31 with a second jaw removed. FIG. 33 is a schematic perspective view of the end tool of FIG. 32 with a cartridge removed. FIG. 34 is a perspective view schematically illustrating a first jaw of the end tool of FIG. 31. FIG. 35 is a plan view of FIG. 34 viewed in one direction. FIG. 36 is a front view of FIG. 34 viewed in one direction. FIG. 37 is a perspective view illustrating an operation member of the end tool of FIG. 31. FIG. 38 is a perspective view of the operation member of FIG. 37 viewed from another direction. FIG. 39 is a perspective view for describing a support guide of the operation member of FIG. 37. FIG. 40 is a perspective view illustrating a wedge of the end tool of FIG. 31. FIG. 41 is a front view of FIG. 40. FIG. 42 is a schematic perspective view for describing the operation member, a fixed pulley, and a forward-moving wire of the end tool of FIG. 31. FIG. 43 is a schematic perspective view illustrating a state in which the first jaw is removed from FIG. 42.

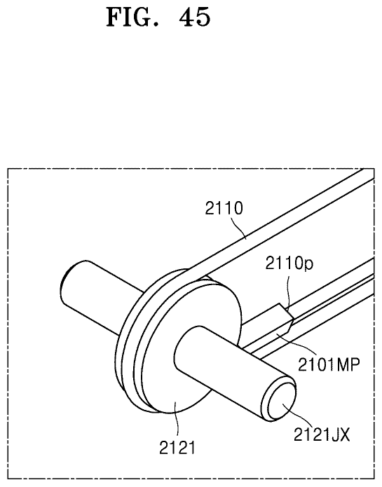

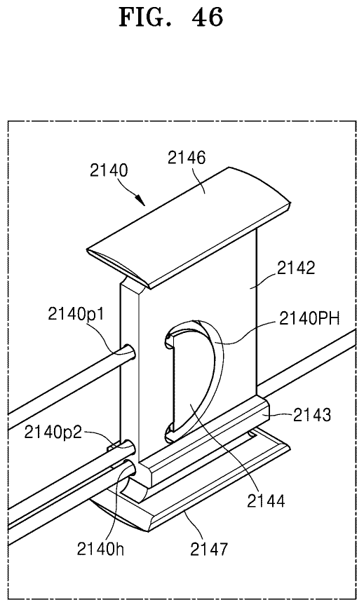

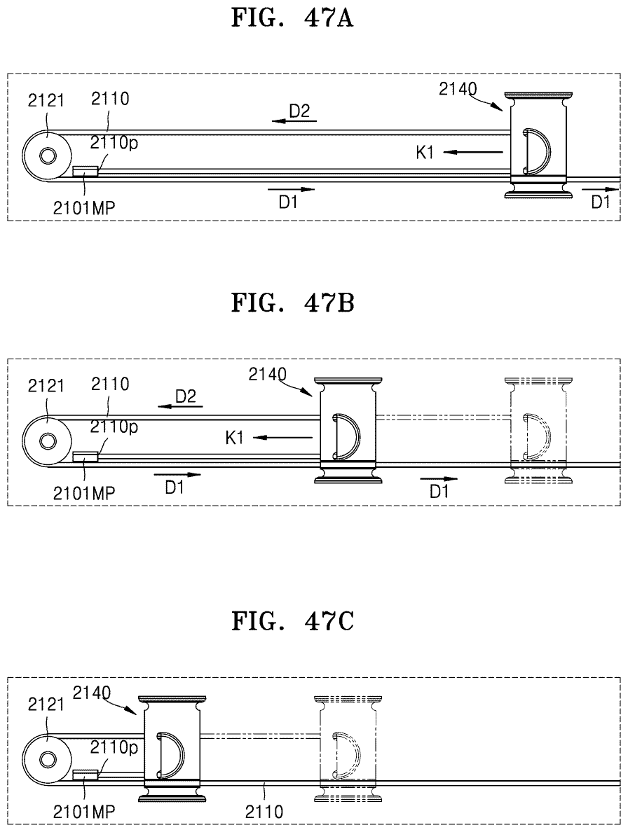

FIG. 44 is a side view of FIG. 43 viewed in one direction. FIGS. 45 and 46 illustrate enlarged views of different regions of FIG. 44, respectively. FIGS. 47A, 47B, and 47C are schematic perspective views for describing an operation of the operation member of the end tool of FIG. 31.

An end tool 2100 of a surgical instrument according to the present embodiment may be applied to various surgical instruments and used, for example, may be applied to surgical instruments including the operator 1200 and the connector 1400, such as the surgical instrument 1000 of the above-described embodiment, and to other surgical instruments of various structure, either as it is or with modifications within the same scope as needed.

Referring to FIGS. 31 to 33, an end tool 2100 of the present embodiment may include a jaw 2103, a fixed pulley 2121, a support guide 2144, and a forward-moving wire 2110.

The jaw 2103 may perform various functions, for example, a grip motion, and may include a pair of jaws, e.g., a first jaw 2101 and a second jaw 2102 as a specific example. Here, each of the first jaw 2101 and the second jaw 2102, or a component encompassing the first jaw 2101 and the second jaw 2102 may be referred to as the jaw 2103.

The first jaw 2101 and the second jaw 2102 may be disposed to face each other, may move closer to or move away from each other, and may be formed to rotationally move around, for example, one shaft JX.

A cartridge 2500 may be disposed to be accommodated in the first jaw 2101, and a plurality of staples are disposed inside the cartridge 2500. When an operation member 2140 receives a force through the forward-moving wire 2110 while the first jaw 2101 and the second jaw 2102 are close to each other, such as when the first jaw 2101 and the second jaw 2102 are closed with the body tissue interposed therebetween, the operation member 2140 may push and raise the staples while moving toward a distal end 2101d of the first jaw 2101, so that stapling may be performed. At this point, one or more clamps 2146 of the operation member 2140 may protrude to the outside of the first jaw 2101 or the second jaw 2102, allowing the operation member 2140 to move forward while applying pressure to an outer surface of the first jaw 2101 or the second jaw 2102 (an upper surface of the second jaw based on FIG. 31), which facilitates the smooth progression of a stapling process.

Meanwhile, the operation member 2140 may be used together with a wedge WDG. For example, the wedge WDG may be prepared separately from the operation member 2140 and then disposed adjacent to the operation member 2140 in the first jaw 2101. In addition, as another example, the operation member 2140 and the wedge WDG may be integrally formed. The wedge WDG may be disposed on at least one side of a body 2145 and may be formed to have a predetermined inclined surface. That is, the wedge WDG may be formed to be inclined by a certain degree with respect to the extension direction of the end tool 2100. In other words, the wedge WDG may be formed to have a greater height at a proximal end 2101p side of the first jaw 2101 than a distal end 2101d side of the first jaw 2101.

The wedge WDG may be formed to be sequentially in contact with withdrawal members (refer to 1535 of FIG. 28) or a plurality of staples (refer to 1530 of FIG. 28) disposed in the cartridge 2500, and may serve to sequentially push and raise the staples 1530 (refer to FIG. 28). More detailed description will be provided below.

The second jaw 2102 is formed in an elongated bar shape as a whole, for example, the second jaw 2102 may be formed in a rod shape to correspond to the first jaw 2101 in at least one region, and the second jaw 2102 is substantially identical to the second jaw 1102 of the above-described embodiment, and thus a further detailed description will be omitted.

The first jaw 2101 will now be described.

FIG. 34 is a perspective view schematically illustrating the first jaw of the end tool of FIG. 31. FIG. 35 is a plan view of FIG. 34 viewed in one direction.

FIG. 36 is a front view of FIG. 34 viewed in one direction.

Referring to FIGS. 34 to 36 and the like, the first jaw 2101 may be formed in an elongated bar shape as a whole, and the cartridge 2500 (refer to FIG. 31) may be accommodated in the first jaw 2101 at the distal end 2101d side. A rotation shaft may be disposed in the proximal end so that the first jaw 2101 is rotationally movable therearound, and the rotation shaft may correspond to the rotation shaft formed in the second jaw 2102.

The first jaw 2101 may entirely be formed in the form of a hollow box with one surface (upper surface) thereof is removed, such that a cartridge accommodation part 2101a capable of accommodating the cartridge 2500 may be formed inside the first jaw 2101. That is, the first jaw 2101 may be formed in an approximately “U” shape in cross section.

A guide groove 2101h may be formed in a bottom surface of the first jaw 2101, the bottom surface opposite to an upper open region formed by removing one surface. Specifically, the guide groove 2101h may be formed to guide a linear motion of the operation member 2140.

The guide groove 2101h may be formed to guide the operation member 2140, and may be a groove formed to pass through a region facing the operation member 2140. Through this, one region of the operation member 2140, such as at least one region of a body 2142 of the operation member 2140, or a second clamp 2147 connected thereto may pass through the guide groove 2101h to exit to the outside of the first jaw 2101. When the operation member 2140 moves forward, the second clamp 2147 may pass through the guide groove 2101h of the first jaw 2101 to be exposed to the outside of the first jaw 2101, and may come into contact with the lower surface of the first jaw 2101 or apply pressure thereto. As the operation member 2140 moves, the second clamp 2147 applies pressure on the lower surface of the first jaw 2101 and a first clamp 2146 applies pressure on the upper surface of the second jaw 2102 such that a gap between the second jaw 2102 and the first jaw 2101 decreases, allowing the second jaw 2102 to naturally remain in a closed state with respect to the first jaw 2101.

In an optional embodiment, the first jaw 2101 may include a window 2101b. After operating the operation member 2140 or using the end tool 2100, the second clamp 2147 of the operation member 2140 may be located corresponding to the window 2101b, and the coupled state of the first jaw 2101 and the operation member 2140 may be released.

The first jaw 2101 may include an extension groove 2101w in the bottom surface thereof. The extension groove 2101w may be formed in the bottom surface of the first jaw 2101, i.e., in the bottom surface on which the cartridge accommodation part 2101a accommodating the cartridge therein is formed.

The extension groove 2101w may be formed to overlap a movement path of the operation member 2140, and may have a predetermined length in the longitudinal direction of the first jaw 2101. For example, the extension groove 2101w may be formed to have a length equal to a length of the first jaw 2101.

In an optional embodiment, the extension groove 2101w may be formed to overlap the guide groove 2101h. For example, the extension groove 2101w may be formed in such a shape in which one region of the bottom surface of the first jaw 2101 in which the cartridge accommodation part 2101a is formed is recessed, and the guide groove 2101h overlapping the extension groove 2101w and having a width less than or equal to a width of the extension groove 2101w may be formed to pass through the bottom surface of the first jaw 2101.

Accordingly, the arrangement and motion of the forward-moving wire 2110 and the arrangement and motion of the operation member 2140 may be efficiently controlled.

A protrusion 2101M may be formed on the extension groove 2101w to be closer to the distal end 2101d than the guide groove 2101h, and may protrude to have a height from the bottom of the extension groove 2101w.

The protrusion 2101M may include a fixed region 2101MP and a pass-through region 2101MT. The fixed region 2101MP may include a region to which one end portion, e.g., one end portion region 2110p, of the forward-moving wire 2110 is fixed. For example, the fixed region 2101MP is formed in the shape of a pass-through part through which one end portion of the forward-moving wire 2110 is inserted or passes, and the forward-moving wire 2110 may be fixed after passing through the pass-through part. As a specific example, a crimp member may be attached or fixed to the end portion of the forward-moving wire 2110, or alternatively, one region of the forward-moving wire 2110 may be fixed to the fixed region 2101MP, through which the forward-moving wire 2110 passes, by various methods such welding, gluing, or the like instead of attaching the crimp member.

The pass-through region 2101MT may have the form of a pass-through part having a width greater than that of the forward-moving wire 2110 so that at least one region of the forward-moving wire 2110 moves therethrough.

One end portion of the forward-moving wire 2110 extends in a direction toward the distal end 2101d from the proximal end 2101p of the first jaw 2101, passes through a wire path 2140h (refer to FIGS. 37 to 46) of the operation member 2140, and is directed to the fixed pulley 2121. At this point, the forward-moving wire 2110 passes through the pass-through region 2101MT of the protrusion 2101M, emerges from an upper side of the fixed pulley 2121 after being wound around a lower side of the fixed pulley 2121 to extend in a direction toward the proximal end 2101p from the distal end 2101d of the first jaw 2101. Thereafter, the forward-moving wire 2110 emerges from a lower side of the support guide 2144 of the operation member 2140 after being wound around the support guide 2144 and then extends again toward the distal end 2101d from the proximal end 2101p of the first jaw 2101. Then, the one end portion region 2110p of the forward-moving wire 2110 is fixed to the fixed region 2101MP of the protrusion 2101M. Accordingly, when the forward-moving wire 2110 is pulled, the operation member 2140 may move forward, which will be described in more detail later.

In addition, in order to efficiently achieve and control such arrangement and movement of the forward-moving wire 2110, the fixed region 2101MP of the protrusion 2101M may be disposed above the pass-through region 2101MT. For example, the pass-through region 2101MT of the protrusion 2101M may be disposed closer to the bottom surface of the first jaw 2101 or a bottom surface of the extension groove 2101w than the fixed region 2101MP.

Since the fixed region 2101MP is disposed above the pass-through region 2101MT, the fixed pulley 2121 may increase in diameter. For example, since the lower end of the fixed pulley 2121 and the pass-through region 2101MT need to be on overlapping, identical, or similar lines, the fixed pulley 2121 may have a diameter that extends up to the pass-through region 2101MT located below the fixed region 2101MP, rather than extending up to the fixed region 2101MP.

In addition, since the fixed region 2101MP is disposed above the pass-through region 2101MT, interference that may affect the forward-moving wire 2110 may be reduced or prevented. This is because that, for example, in the opposite case where the fixed region 2101MP is located below the pass-through region 2101MT, the forward-moving wire 2110 passes through the fixed region 2101MP, the operation member 2140, the fixed pulley 2121, the operation member 2140, and the fixed pulley 2121, in that order, and thus, there may be overlap in the forward-moving wire 2110, resulting in interference.

The operation member 2140 will now be described in detail.

FIG. 37 is a perspective view illustrating the operation member of the end tool of FIG. 31. FIG. 38 is a perspective view of the operation member of FIG. 37 viewed from another direction. FIG. 39 is a perspective view for describing the support guide of the operation member of FIG. 37. FIG. 40 is a perspective view illustrating the wedge of the end tool of FIG. 31. FIG. 41 is a front view of FIG. 40.

The operation member 2140 may include the body 2142, the first clamp 2146, and the second clamp 2147. Meanwhile, the operation member 2140 may be used together with the wedge WDG. For example, the wedge WDG may be prepared separately from the operation member 2140 and then disposed adjacent to the operation member 2140 in the first jaw 2101. In addition, as another example, the operation member 2140 and the wedge WDG may be integrally formed. In the present specification, for convenience of description, the operation member 2140 and the wedge WDG will be described and illustrated in the drawings with the assumption that the operation member 2140 and the wedge WDG are prepared separately.

The wedge WDG may be disposed on at least one side of the body 2142 and may be formed to have a predetermined inclined surface WDG1. That is, the wedge WDG may be formed to be inclined by a certain degree with respect to the extension direction of the end tool 2100. In other words, the wedge WDG may be formed to have a greater height at a proximal end 2101p side of the first jaw 2101 than a distal end 2101d side of the first jaw 2101.

Meanwhile, a groove WDGh may be formed in a lower surface of the wedge WDG, and at least one region of the forward-moving wire 2110, such as at least a portion of the region of the forward-moving wire 2110, which emerges from being wound around the support guide 2144 of the operation member 2140 and extends toward the fixed region 2101MP, may correspond to the groove WDGh.

The wedge WDG may be formed to be in contact with the withdrawal members (refer to 1535 of FIG. 28 described above) or the plurality of staples (refer to 1530 of FIG. 28 described above), and may serve to sequentially push and raise the staples, and a detailed description thereof is substantially the same as that described in the above-described embodiment, and thus a detailed description thereof will be omitted.

The body 2142 may be in the form of an elongated column, such as a plate-shaped column. In addition, a blade region may be formed in one region of the body 2142, and an edge sharply formed to cut tissue may be formed in the blade region. The tissue disposed between the first jaw 2101 and the second jaw 2102 may be cut as at least a portion of the edge formed in one region of the body 2142 is withdrawn to the outside of the first jaw 2101 and the cartridge 2500.

The first clamp 2146 may be formed in one region of the body 2142, and the second clamp 2147 may be formed in another region different from the one region. For example, the body 2142 may be disposed between the first clamp 2146 and the second clamp 2147.

The first clamp 2146 and the second clamp 2147 may be formed to have a width at least greater than that of the body 2142.

When the operation member 2140 moves from the proximal end 2101p of the first jaw 2101 toward the distal end 2101d of the first jaw 2101, a motion of decreasing a distance between the second jaw 2102 and the first jaw 2101, i.e., a closing motion of the jaw 2103, may be naturally implemented through the first clamp 2146 and the second clamp 2147, and the specific contents of the first clamp 2146 and second clamp 2147 and the effects thereof are substantially the same as those described in the above-described embodiment, and thus detailed descriptions thereof will be omitted.

A support guide 2144 may be formed in one region of the body 2142. For example, the support guide 2144 may be formed on an inner side of the body 2142. As a specific example, the support guide 2144 may be integrally formed with the inner side of the body 2142. The support guide 2144 may include a groove 2144g for the forward-moving wire 2110 to be wound therearound.

In an optional embodiment, the support guide 2144 may include a curved outer circumferential surface, for example, a semicircular-shaped outer circumferential surface. At this point, the curved outer circumferential surface or semicircular-shaped outer circumferential surface may be disposed adjacent to the rear side, i.e., closer to the proximal end 2101p than to the distal end 2101d of the first jaw 2101. With this structure, friction that may occur when the forward-moving wire 2110 comes into contact with and is wound around the support guide 2144 may be reduced.

Further, although not shown in the drawings, in an optional embodiment, the support guide 2144 may have a cylindrical shape similar to a typical pulley, and may further include a rotatable rotation shaft. Of course, in this case, since the support guide 2144 moves together with the operation member 2140, the rotation shaft may be formed to be rotatable while being fixed to the operation member 2140 such that the rotation shaft can move together with the operation member 2140.

Further, as an example, the support guide 2144 may be prepared separately and then coupled to a space of the body 2142 instead of being formed integrally with the operation member 2140.

A first path 2140p1 and a second path 2140p2 having a width sufficient for the forward-moving wire 2110 to pass through and spaced apart from each other may be formed in the body 2142 to correspond to the support guide 2144. In addition, a space 2140PH corresponding to the outer circumferential surface of the support guide 2144 may be formed in the body 2142. With this structure, one region of the forward-moving wire 2110 may enter through the first path 2140p1, and exit through the second path 2140p2 while being correspondingly wound around the support guide 2144 at the space 2140PH. In addition, this may also be expressed in the reverse case, i.e., where the forward-moving wire 2110 enters through the second path 2140p2 and exits through the first path 2140p1.

The wire path 2140h may be formed to pass through one region of the body 2142, e.g., the front and rear sides of the body 2142. The wire path 2140h may be formed to have a greater width than the forward-moving wire 2110 so that at least one region of the forward-moving wire 2110 passes therethrough and moves.

The wire path 2140h may be formed to be spaced apart from the support guide 2144.

For example, the wire path 2140h may be disposed below the support guide 2144, and as a specific example, the wire path 2140h may be disposed closer to the bottom of the operation member 2140, such as the second clamp 2147, than the support guide 2144.

Accordingly, each of regions of the forward-moving wire 2110 may be efficiently disposed to efficiently transmit a force to the operation member 2140.

In an optional embodiment, the operation member 2140 may include a side part 2143.

The side part 2143 is formed in one region of the body 2142 or a region connected to the body 2142, and is a region corresponding to the wire path 2140h, and may have a greater width than at least the body 2142 and have a shape protruding from both sides of the body 2142 in a width direction as a specific example. By forming the side part 2143 to be wider than the body 2142, a decrease in rigidity of the operation member 2140 due to the width of the operation member 2140 being reduced by the wire path 2140h may be effectively prevented. Accordingly, the operation member 2140 may be improved in durability when moving using the forward-moving wire 2110.

The forward-moving wire 2110 and the fixed pulley 2121 for driving the operation member 2140 will now be described.

FIG. 42 is a schematic perspective view for describing the operation member, the fixed pulley, and the forward-moving wire of the end tool of FIG. 31. FIG. 43 is a schematic perspective view illustrating a state in which the first jaw is removed from FIG. 42. FIG. 44 is a side view of FIG. 43 viewed in one direction. FIGS. 45 and 46 illustrate enlarged views of different regions of FIG. 44, respectively.

The fixed pulley 2121 and the forward-moving wire 2110 may be disposed to move the operation member 2140.

The fixed pulley 2121 may be disposed in the first jaw 2101, for example, may be connected to a pulley shaft 2121JX formed in a direction traversing the width direction of the first jaw 2101, and may be coupled to or integrally formed with the pulley shaft 2121JX. In an optional embodiment, the fixed pulley 2121 may be formed distinct from the pulley shaft 2121JX so as to rotationally move around the pulley shaft 2121JX.

The forward-moving wire 2110 may be wound around an outer circumferential surface of the fixed pulley 2121, and to this end, a groove may be formed on the outer circumferential surface of the fixed pulley 2121.

The fixed pulley 2121 may be disposed closer the distal end 2101d of the first jaw 2101 at least than the operation member 2140.

The fixed pulley 2121 may have a vertically disposed shape, for example, may be disposed parallel to a direction from the first jaw 2101 to the second jaw 2102, and may be disposed to correspond to a center line of the first jaw 2101.

At least one region, such as a lower region, of the fixed pulley 2121 may be disposed to correspond to the extension groove 2101w of the first jaw 2101.

Since the forward-moving wire 2110 is correspondingly wound around the fixed pulley 2121, one region of the forward-moving wire 2110 may also be disposed to correspond to the extension groove 2101w of the first jaw 2101.

Accordingly, the forward-moving wire 2110 and the fixed pulley 2121 may be efficiently disposed while easily securing a space for moving the operation member 2140 of the first jaw 2101 and stapling.

The forward-moving wire 2110 may extend to have a length in the longitudinal direction of the first jaw 2101. In addition, one end portion region of the forward-moving wire 2110 may extend to the proximal end 2101p of the first jaw 2101 to be connected to the inside of a driving part, such as an operator (refer to, for example, 1200 of FIG. 1), and pulled through the manipulation of the operator.

Another end portion of the forward-moving wire 2110 extends in a direction toward the distal end 2101d of the first jaw 2101 in the longitudinal direction of the first jaw 2101, passes through the pass-through region 2101MT (refer to FIGS. 34 to 36) after passing through the wire path 2140h of the operation member 2140, and then, emerges from the upper side of the fixed pulley 2121 after being wound around the lower side of the fixed pulley 2121 to extend in a direction toward the proximal end 2101p of the first jaw 2101. Thereafter, the another end portion of the forward-moving wire 2110 enters through the first path 2140pl of the operation member 2140, enters the upper side of the support guide 2144 at the space 2140PH corresponding to the support guide 2144, emerges from the lower side of the support guide 2144 while being wound around the support guide 2144, and then exits through the second path 2140p2 to extend in a direction toward the distal end 2101d of the first jaw 2101. Then, the another end portion of the forward-moving wire 2110 is fixed to the fixed region 2101MP of the protrusion 2101M.

Further, each region of the forward-moving wire 2110 of the present embodiment is disposed so as not to deviate from the regions at which the forward-moving wire 2110 enters and is wound around the fixed pulley 2121, and the region at which the forward-moving wire 2110 emerges from the fixed pulley 2121. For example, the forward-moving wire 2110 may be disposed not to deviate from the width of the fixed pulley 2121.

As a specific example, the region of the forward-moving wire 2110, which emerges from being wound around the support guide 2144 and is directed toward the fixed region 2101MP, is placed between the region of the forward-moving wire 2110, which passes through the wire path 2140h and is directed toward the lower side of the fixed pulley 2121, and the region of the forward-moving wire 2110, which emerges from being wound around the upper side of the fixed pulley 2121 and is directed toward the operation member 2140.

By compactly arranging the forward-moving wire 2110, the efficiency of arranging the forward-moving wire 2110 may be improved, and a force pulling the forward-moving wire 2110 may be effectively transmitted to the operation member 2140.

In such an arrangement, the operation member 2140 may be controlled to move forward by half the distance the forward-moving wire 2110 is pulled, thereby allowing the operation member 2140 to be pulled with a relatively small force.

In an optional embodiment, the region of the forward-moving wire 2110, which extends toward the distal end 2101d of the first jaw 2101, passes through the wire path 2140h of the operation member 2140, and then directs toward the lower side of the fixed pulley 2121, may be parallel to the region of the forward-moving wire 2110, which emerges from the upper side of the fixed pulley 2121, extends toward the proximal end 2101p of the first jaw 2101, and directs toward the support guide 2144 of the operation member 2140, and the region of the forward-moving wire 2110 emerges from the lower side of the support guide 2144 and directs toward the fixed region 2101MP.

Accordingly, when pulling the forward-moving wire 2110 toward the proximal end 2101p, a pulling force may be effectively transmitted to the operation member 2140, and the distance the forward-moving wire 2110 is pulled and the distance the operation member 2140 moves forward may be precisely controlled. In addition, as an example, when pulling the forward-moving wire 2110 toward the proximal end 2101p, a ratio of the pulling force to the corresponding forward-moving distance of the operation member 2140 may be precisely controlled at a predetermined ratio. As a specific example, when pulling the forward-moving wire 2110 toward the proximal end 2101p, the ratio of the pulling force to the forward-moving distance of the operation member 2140 may be easily controlled to a value equal to, or substantially equal or similar to 1:1.

Accordingly, as shown in FIGS. 43 and 44, when the forward-moving wire 2110 is pulled in a first direction D1, one region of the forward-moving wire 2110 passes through the wire path 2140h of the operation member 2140 and moves in the first direction D1. Thus, the region of the forward-moving wire 2110, which is wound around the lower side of the fixed pulley 2121 and emerges from the upper side of the fixed pulley 2121, receives a force to move in a second direction D2, which is opposite to the first direction D1, and the region of the forward-moving wire 2110, which is fixed to the fixed region 2101MP after emerging from being wound around the support guide 2144, receives a force to move in the first direction D1. This movement of the forward-moving wire 2110 causes the forward-moving wire 2110 to receive a force that causes a distance between the support guide 2144 and the fixed region 2101MP to decrease, and due to this force, the operation member 2140 moves in a direction K1, which is the same direction as the second direction D2, i.e., moves forwards.

FIGS. 47A, 47B, and 47C are schematic perspective views for describing an operation of the operation member of the end tool of FIG. 31.

Referring to FIG. 47A, the first jaw 2101 is excluded for convenience of description, and the forward-moving wire 2110, the fixed pulley 2121, and the operation member 2140 are illustrated.

As shown in FIG. 47A, when the forward-moving wire 2110 is pulled in the first direction D1, one region of the forward-moving wire 2110 passes through the wire path 2140h of the operation member 2140 and is pulled in the first direction D1, so that, the region of the forward-moving wire 2110, which emerges from the upper side of the fixed pulley 2121 after being wound around the lower side of the fixed pulley 2121, receives a force in the direction of moving in the second direction D2, which is opposite to the first direction D1, and accordingly, the region of the forward-moving wire 2110, which enters the upper side of the support guide 2144 and emerges from the lower side of the support guide 2144, receives a force in the first direction D1. Since one end portion of the forward-moving wire 2110 is fixed to the fixed region 2101MP, such a movement of the forward-moving wire 2110 causes the forward-moving wire 2110 to receive a force that causes a distance between the support guide 2144 and the fixed region 2101MP to decrease, and due to the force, the operation member 2140 moves in the direction K1, which is the same direction as the second direction D2, i.e., moves forwards, thereby positioning the operation member 2140 in an advanced position shown in FIG. 47B.

Thereafter, as shown in FIG. 47B, when the forward-moving wire 2110 is further pulled in the first direction D1, one region of the forward-moving wire 2110 passes through the wire path 2140h of the operation member 2140 and is pulled further in the first direction D1, so that, the region of the forward-moving wire 2110, which emerges from the upper side of the fixed pulley 2121 after being wound around the lower side of the fixed pulley 2121, receives a force in the direction of moving further in the second direction D2, which is opposite to the first direction D1, and accordingly, the region of the forward-moving wire 2110, which enters the upper side of the support guide 2144 and emerges from the lower side of the support guide 2144, further receives the force in the first direction D1. Such a movement of the forward-moving wire 2110 causes the forward-moving wire 2110 to receive a force that causes a distance between the support guide 2144 and the fixed region 2101MP to decrease, and due to the force, the operation member 2140 moves in the direction K1, which is the same direction as the second direction D2, i.e., moves forwards, causing the operation member 2140 to move forward to a position further forward than that in FIG. 47B, as shown in FIG. 47C.

Although not shown in the drawings, the above-described state described in FIGS. 20A, 20B, and 20C may also be applied to the end tool 2100 of the present embodiment.

In addition, although not shown in the drawings, the modified example of the fixed pulley having an inclined shape of FIGS. 21 to 24 described above may be optionally applied to the end tool 2100 of the present embodiment.

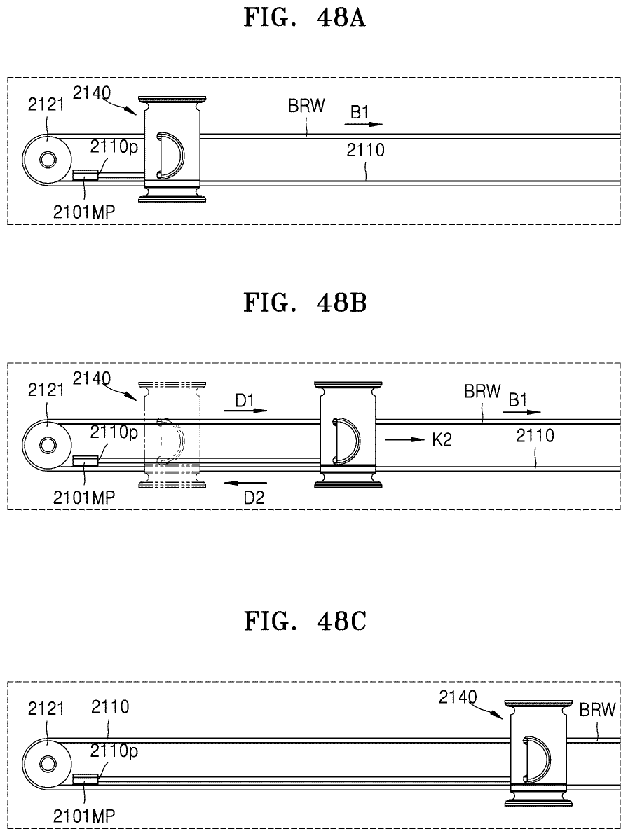

FIGS. 48A, 48B, and 48C are views for describing an optional embodiment in which a backward-moving wire is added to the end tool of FIG. 31.

Referring to FIGS. 48A, 48B, and 48C, the end tool of the present embodiment may further include a backward-moving wire BRW. The backward-moving wire BRW may be connected to one region of the operation member 2140 and may be connected, for example, to a rear side of the operation member, specifically, to a surface of the operation member 2140, which is opposite to the surface of the operation member 2140 facing the fixed pulley 2121.

A driving part or driving transmission part (e.g., a wire, a pulley, or the like) capable of pulling the backward-moving wire BRW may be connected to the backward-moving wire BRW, and the backward-moving wire BRW may be operated according to manual or automatic manipulation. For example, the backward-moving wire BRW may be pulled by the operator (refer to, for example, 1200 of FIG. 1).

By pulling the backward-moving wire BRW. The operation member 2140 may move backward.

For example, as shown in FIG. 48A, the backward-moving wire BRW is pulled in a reverse direction B1 in a state in which the operation member 2140 is located adjacent to the distal end 2101d of the first jaw 2101, the operation member 2140 connected to the backward-moving wire BRW moves backward in a direction K2, which is the same direction as the reverse direction B1. At this point, the forward-moving wire 2110 may be in a state where no pulling force is applied. Accordingly, when the backward-moving wire BRW moves, the operation member 2140 connected to the backward-moving wire BRW may stably move backward without interference from the forward-moving wire 2110. Accordingly, the operation member 2140 is in a position as shown in FIG. 48B, having moved backward to be closer to the proximal end 2101p compared to its position in FIG. 48A.

Thereafter, as shown in FIG. 48B, when the backward-moving wire BRW is pulled further in the reverse direction B1, the operation member 2140 connected to the backward-moving wire BRW moves backward in the direction K2, which is the same direction as the reverse direction B1. At this point, since the forward-moving wire 2110 may be in a state in which no pulling force is continuously applied, when the operation member 2140 moves backward (in the direction K2), the operation member 2140 connected to the backward-moving wire BRW may move forward in the direction K2, and thus the operation member 2140 is in a position as shown in FIG. 48C, having moved backward to be closer to the proximal end 2101p compared to its position in FIG. 48B. In the present embodiment, distances moved by the forward-moving wire 2110 and the backward-moving wire BRW may be different when the same force is applied, and specifically, the backward-moving wire BRW may move further, for example, a ratio of the moved distances may be a 1:2. In this case, pulleys that transmit driving forces to the forward-moving wire 2110 and the backward-moving wire BRW may be separately disposed.

In an optional embodiment, the forward-moving wire 2110 and the backward-moving wire BRW may be connected to a single common pulley (not shown) by adding a correction member that corrects for these different moving distances to be equal or nearly equal to each other. For example, when a pulley member is added or applied to the backward-moving wire BRW, similarly to the forward-moving wire 2110, such that a system is implemented so that the operation member 2140 moves ½ as much as pulling the backward-moving wire BRW, it is also possible to connect the forward-moving wire 2110 and the backward-moving wire BRW to a common pulley to form a loop. As a specific example, the pulley member may be implemented in the operation member 2140 or on the operation member 2140, or may be implemented adjacent to the common pulley. When the pulley member implemented adjacent to the common pulley is applied, one or more auxiliary pulleys may be added to safely implement the path.

In an optional embodiment, the forward-moving wire 2110 and the backward-moving wire BRW may not be both placed in a central plane at least the proximal end 2101p of the end tool 2100. The path of the forward-moving wire 2110 or the backward-moving wire BRW may be controlled through a pulley or the like to ensure that the forward-moving wire 2110 or the backward-moving wire BRW is not placed on a plane that crosses the center line of the end tool 2100, thereby facilitating the arrangement of the components for yaw and pitch motions of the end tool 2100.

Although not shown in the drawings, the structure of the above-described embodiment described with reference to FIGS. 26 and 27 may be optionally applied to the end tool 2100 of the present embodiment.

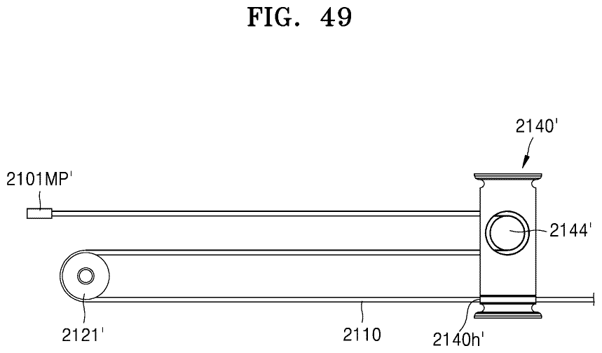

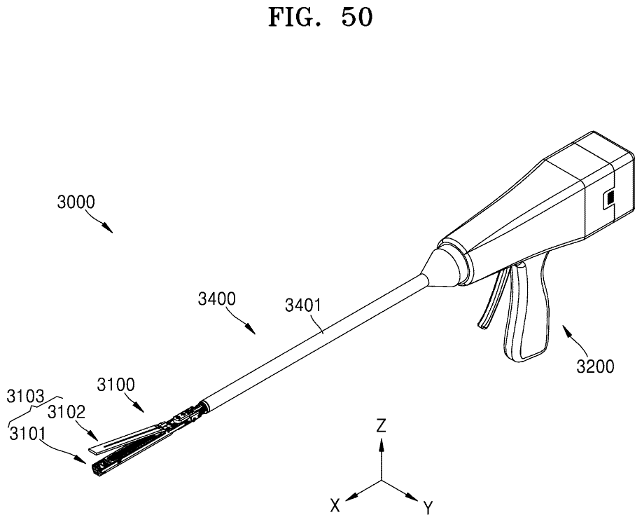

FIG. 49 is a view for describing one modified example of the end tool of FIG. 31.

Referring to FIG. 49, a configuration for arranging the forward-moving wire 2110 and the form of driving the forward-moving wire 2110 are different from those of the end tool 2100 of FIG. 31, and thus mainly described.

The forward-moving wire 2110 may extend to have a length in a longitudinal direction of a first jaw (refer to 2101 of FIGS. 48A, 48B, and 48C). In addition, one end portion region of the forward-moving wire 2110 may extend to the proximal end 2101p of the first jaw (refer to 2101 of FIGS. 48A, 48B, and 48C) to be connected to the inside of a driving part, such as an operator (refer to, for example, 1200 of FIG. 1), and pulled through the manipulation of the operator.

Another end portion of the forward-moving wire 2110 extends in a direction toward the distal end 2101d of the first jaw, passes through a wire path 2140h′ of an operation member 2140′, and then emerges from an upper side of the fixed pulley 2121′ after being wound around a lower side of the fixed pulley 2121′ to extend toward the proximal end 2101p of the first jaw. Thereafter, the another end portion of the forward-moving wire 2110 may emerge from an upper side of a support guide 2144′ after entering and being wound around a lower side of the support guide 2144′, and extend in a direction toward the distal end 2101d of the first jaw 2101 (refer to FIGS. 48A, 48B, and 48C) to be connected and fixed to a fixed region 2101MP′.

That is, in the above-described embodiment, the fixed region 2101MP is disposed between one region of the lower side of the forward-moving wire 2110 wound around the fixed pulley 2121 and one region of the upper side of the forward-moving wire 2110 wound around the fixed pulley 2121, whereas in the present modified example, the fixed region 2101MP′ is disposed further above one region of the lower side of the forward-moving wire 2110 wound around the fixed pulley 2121′ and one region of the upper side of the forward-moving wire 2110 wound around the fixed pulley 2121′.

With such arrangement, the design freedom of the movement path of the forward-moving wire 2110 may be improved, and the design convenience of the position at which the fixed region 2101MP′ configured to fix one end of the forward-moving wire 2110 is disposed in the first jaw 2101 may be improved.

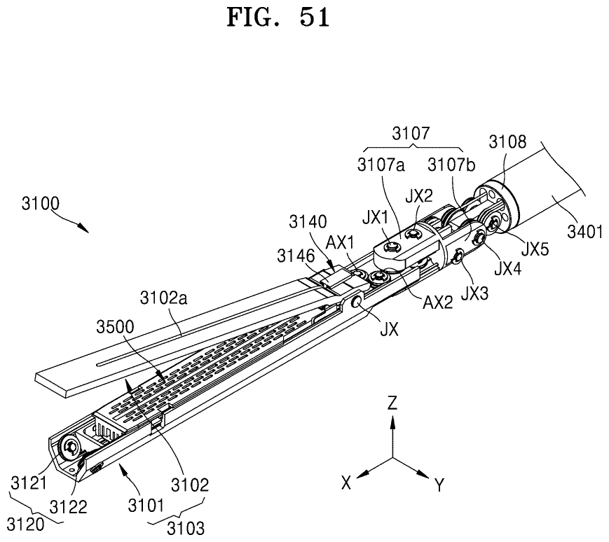

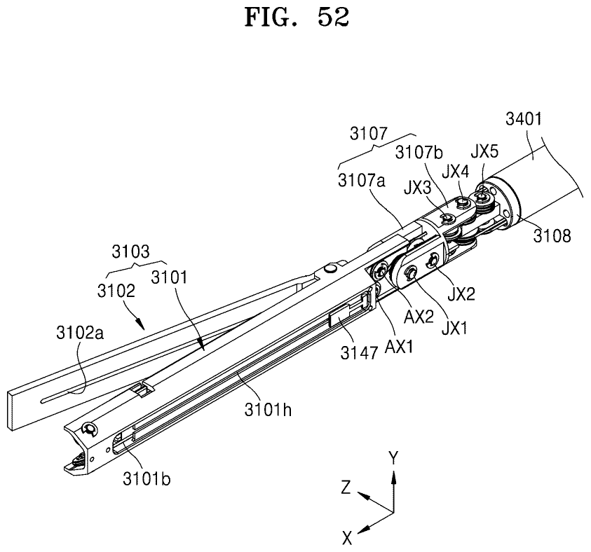

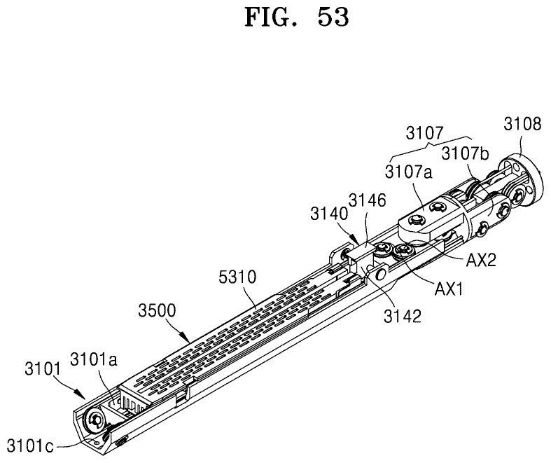

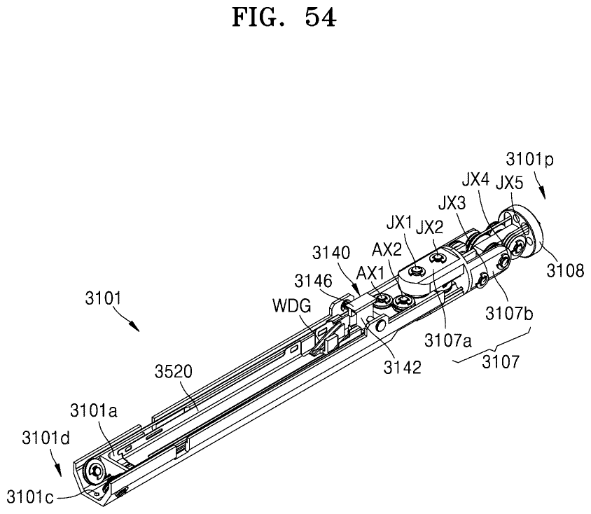

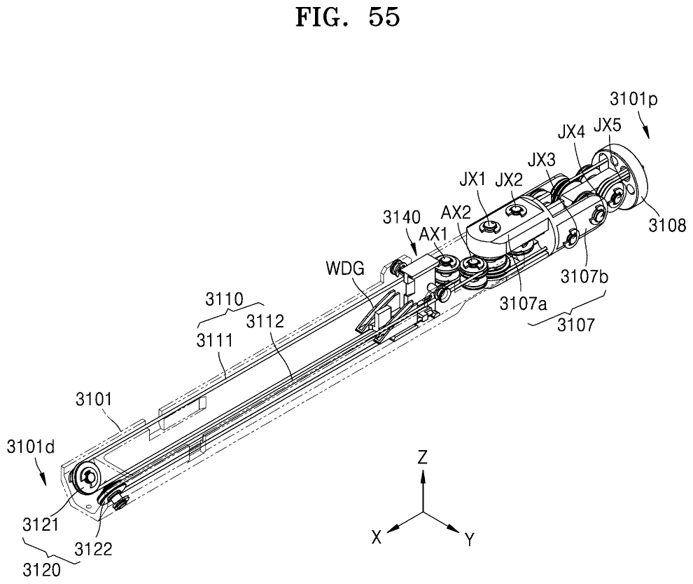

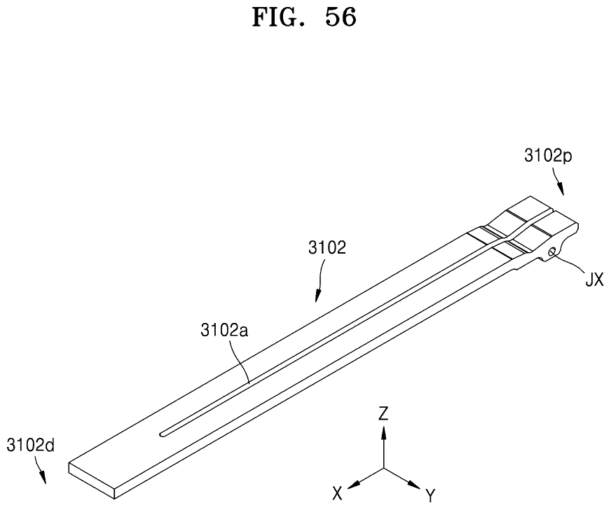

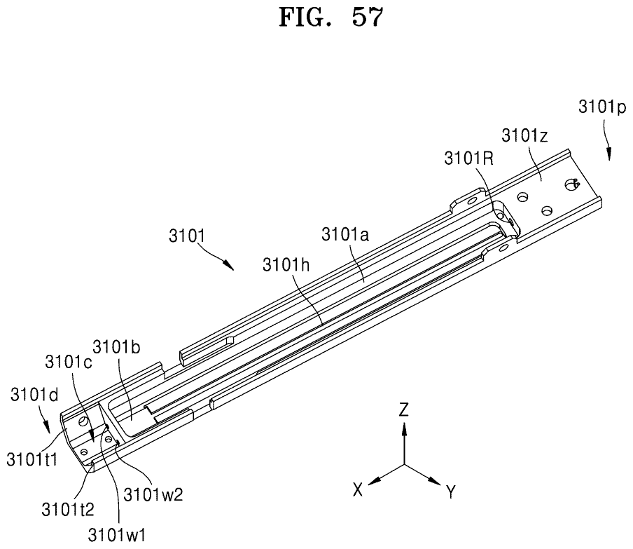

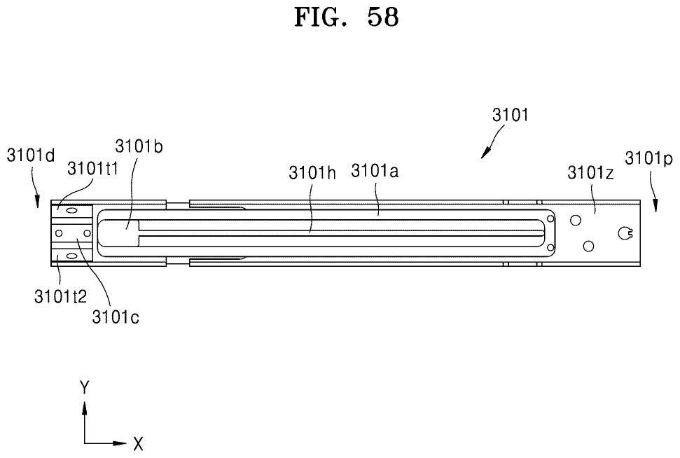

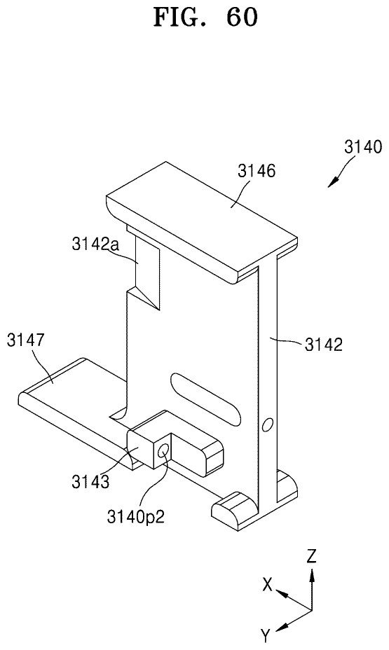

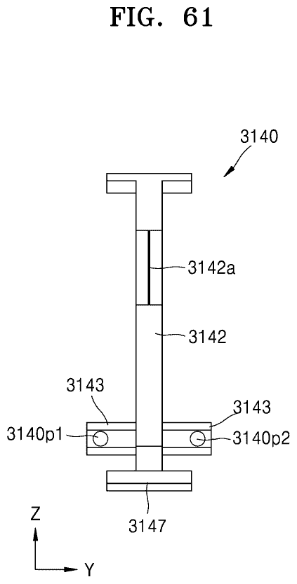

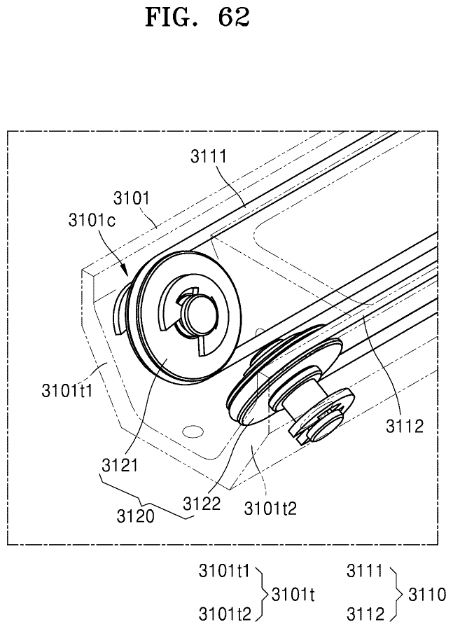

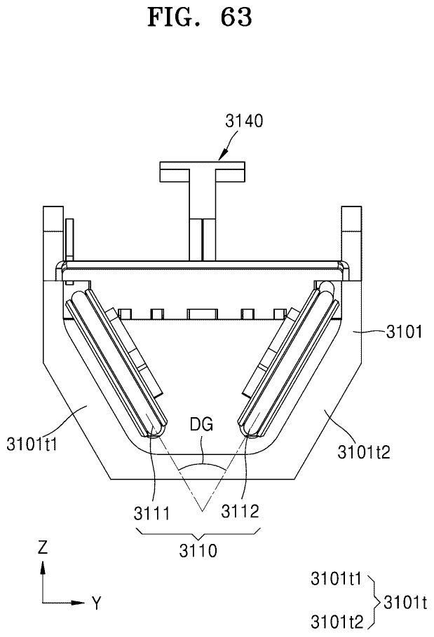

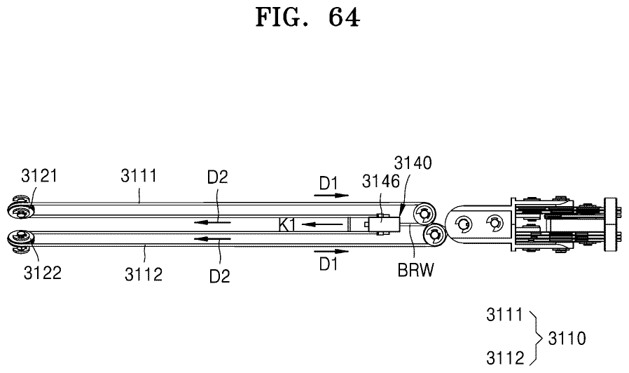

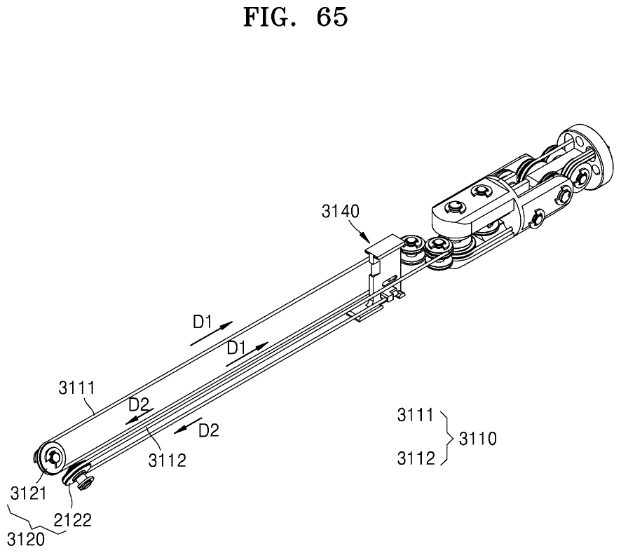

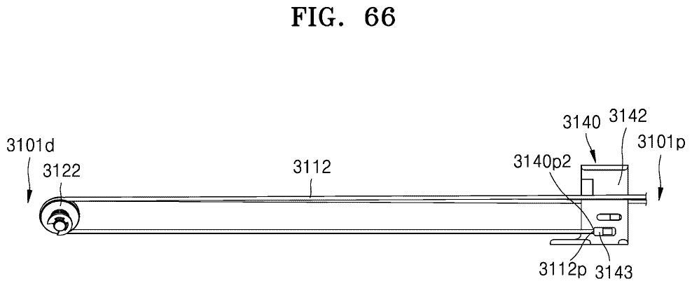

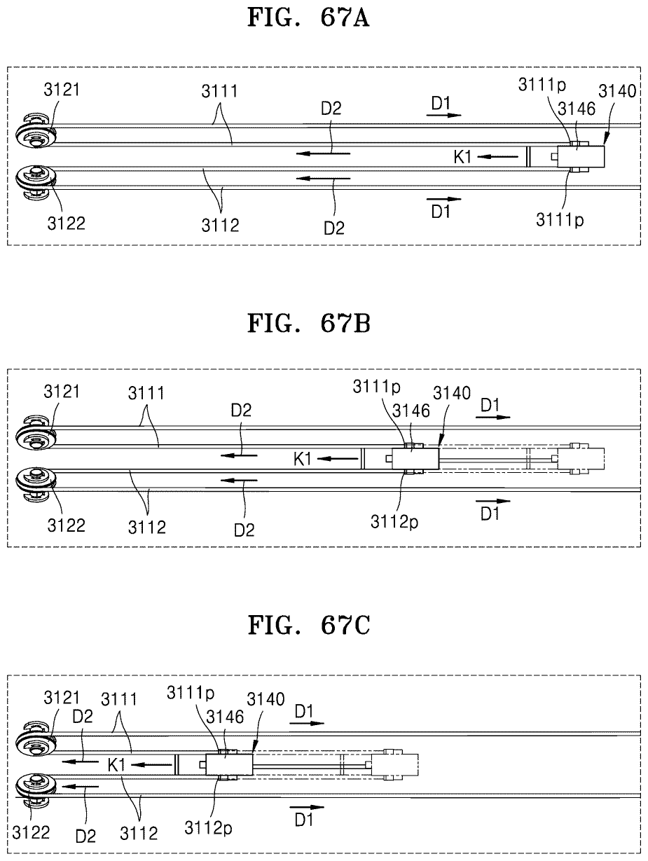

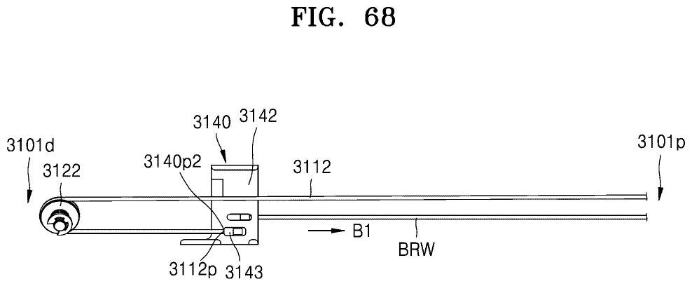

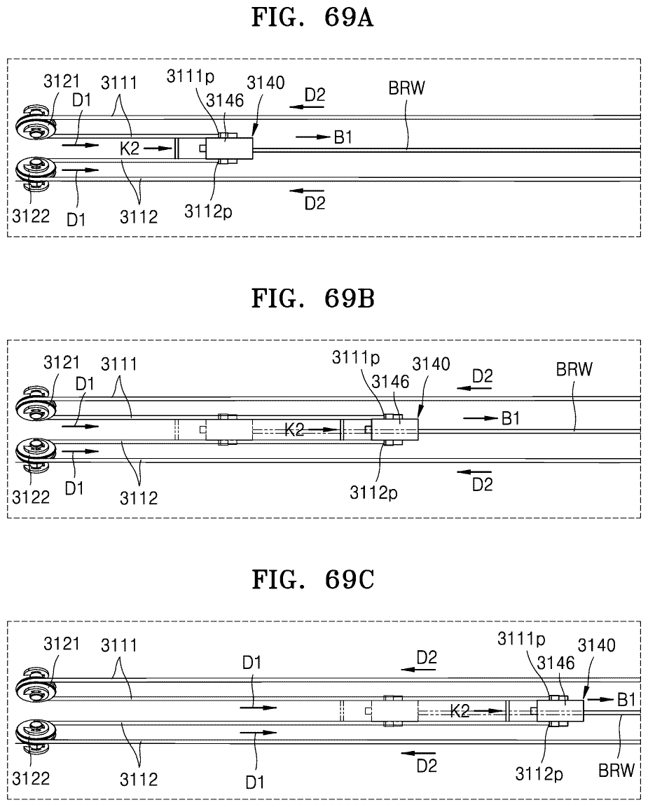

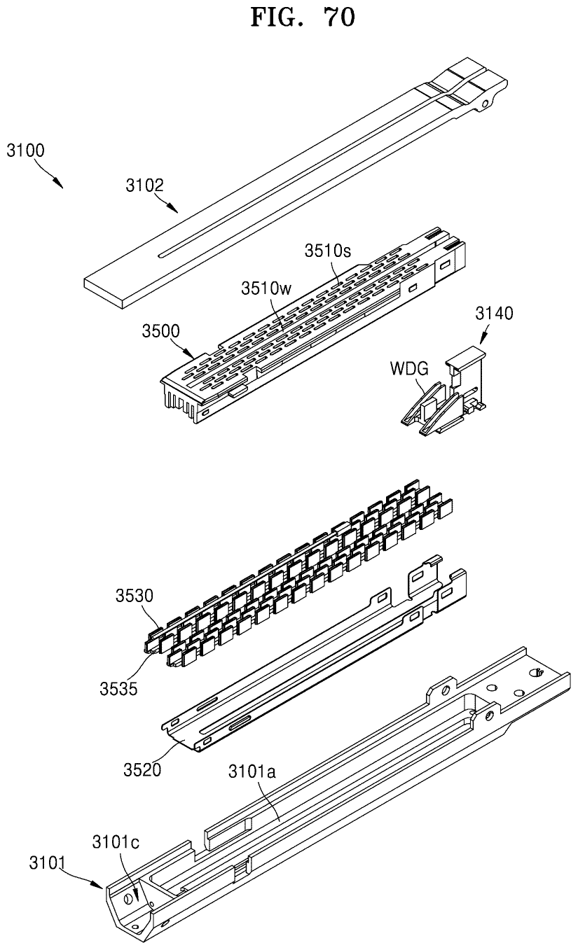

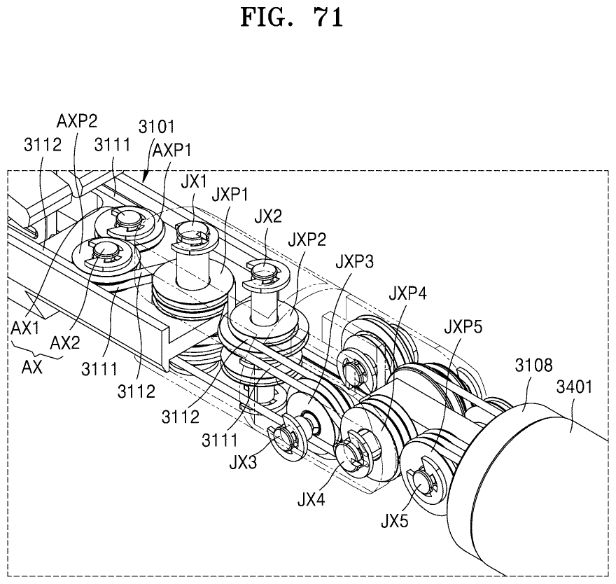

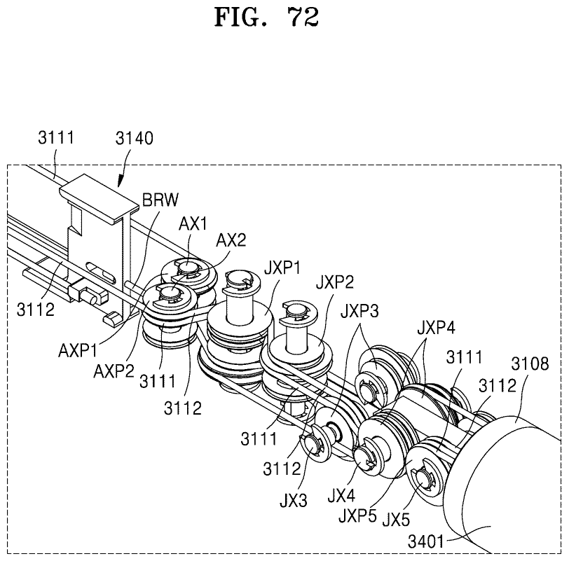

In addition, although not shown in the drawings, one or more pulleys may be further added, thereby efficiently moving the operation member forward while reducing the force pulling the forward-moving wire 2110. For example, by adding additional pulleys that move together with the operation member or in connection with another component, the operation member may move a distance D/X when the wire is pulled a distance D with the addition of X pulleys, thereby increasing the force of the wire pulling the operation member by a factor of X. FIG. 50 is a schematic perspective view of a surgical instrument according to another embodiment of the present disclosure. FIG. 51 is a schematic perspective view for describing an end tool of FIG. 50. FIG. 52 is a perspective view of the end tool of FIG. 51 viewed from another direction. FIG. 53 is a schematic perspective view of the end tool of FIG. 51 with a second jaw removed. FIG. 54 is a schematic perspective view of the end tool of FIG. 53 with a cartridge removed. FIG. 55 is a transparent perspective view of FIG. 54. FIG. 56 is a perspective view schematically illustrating the second jaw of the end tool of FIG. 51. FIG. 57 is a perspective view schematically illustrating a first jaw of the end tool of FIG. 51. FIG. 58 is a plan view schematically illustrating the first jaw of the end tool of FIG. 51. FIG. 59 is a perspective view illustrating an operation member of the end tool of FIG. 51. FIG. 60 is a perspective view of the operation member of FIG. 59 viewed from another direction. FIG. 61 is a front view of the operation member of FIG. 59 viewed in one direction. FIG. 62 is a schematic perspective view illustrating a portion of the end tool of FIG. 51. FIG. 63 is a front view of FIG. 62 viewed in one direction. FIG. 64 is a schematic plan view for describing the operation member, a fixed pulley, and a forward-moving wire of the end tool of FIG. 51. FIG. 65 is a schematic perspective view for describing the operation member, the fixed pulley, and the forward-moving wire of the end tool of FIG. 51. FIGS. 66 and 67A, 67B, and 67C are schematic views for describing an operation of the operation member of the end tool of FIG. 51. FIGS. 68 and 69A, 69B, and 69C are views for describing an optional embodiment in which a backward-moving wire is added to the end tool of FIG. 51. FIG. 70 is a perspective view illustrating the first jaw and a cartridge of the surgical instrument of FIG. 50. FIGS. 71 and 72 are views for describing a switching pulley, a yaw pulley, and a pitch pulley of the end tool of the surgical instrument of FIG. 50.

A surgical instrument 3000 according to the present embodiment may include an end tool 3100, an operator 3200, and a connector 3400.

Here, the connector 3400 is formed in the shape of a hollow shaft, and one or more wires and electric wires may be accommodated therein. The operator 3200 is coupled to one end portion of the connector 3400, the end tool 3100 is coupled to another end portion thereof, and the connector 3400 may serve to connect the operator 3200 to the end tool 3100. As an example, the connector 3400 may include a straight part 3401, and although not shown in the drawings, the connector 3400 may include one or more curved parts to increase ease of use and control the arrangement of components for manipulation.

The operator 3200 is formed at the one end portion of the connector 3400 and provided as an interface to be directly controlled by a medical doctor, for example, a tongs shape, a stick shape, a lever shape, or the like, and when the medical doctor controls the operator 3200, the end tool 3100, which is connected to the interface and inserted into the body of a surgical patient, performs a certain motion, thereby performing surgery. Here, the operator 3200 is illustrated in FIG. 50 as being formed in a handle shape allowing user's fingers to come into close contact therewith and perform one or more motions, such as pulling or pushing, but the concept of the present disclosure is not limited thereto, and various types of manipulation parts that can be connected to the end tool 3100 and manipulate the end tool 3100 may be possible.

The end tool 3100 is formed on another end portion of the connector 3400, and performs necessary motions for surgery by being inserted into a surgical site. As an example of the end tool 3100, a pair of jaws 3103 for performing a grip motion may be used. However, the concept of the present disclosure is not limited thereto, and various devices for performing surgery may be used as the end tool 3100. For example, a configuration of a cantilever cautery may also be used as the end tool. The end tool 3100 is connected to the operator 3200 by an operating force transmitter (not shown, e.g., a wire or the like), and receives a driving force of the operator 3200 through the operating force transmitter to perform a motion necessary for surgery, such as gripping, cutting, suturing, or the like.

Hereinafter, the end tool 3100 of the surgical instrument 3000 of FIG. 50 will be described in more detail.

FIG. 51 is a schematic perspective view for describing the end tool of FIG. 50. FIG. 52 is a perspective view of the end tool of FIG. 51 viewed from another direction. FIG. 53 is a schematic perspective view of the end tool of FIG. 51 with the second jaw removed. FIG. 54 is a schematic perspective view of the end tool of FIG. 53 with the cartridge removed. FIG. 55 is a transparent perspective view of FIG. 54.

The end tool 3100 may include the jaw 3103, a plurality of fixed pulleys 3120, and a plurality of forward-moving wires 3110. The plurality of fixed pulleys 3120 may include two or more pulleys, for example, a first fixed pulley 3121 and a second fixed pulley 3122. The plurality of forward-moving wires 3110 include two or more wires, and may include, for example, a first forward-moving wire 3111 and a second forward-moving wire 3112.

The jaw 3103 may perform various functions, for example, a grip motion, and may include a pair of jaws, e.g., a first jaw 3101 and a second jaw 3102 as a specific example. Here, each of the first jaw 3101 and the second jaw 3102, or a component encompassing the first jaw 3101 and the second jaw 3102 may be referred to as the jaw 3103.

The first jaw 3101 and the second jaw 3102 may be disposed to face each other, may move closer to or move away from each other, and may be formed to rotationally move around, for example, one shaft JX.

A cartridge 3500 may be disposed to be accommodated in the first jaw 3101, and a plurality of staples are disposed inside the cartridge 3500. When an operation member 3140 receives a force through the forward-moving wire 3110 while the first jaw 3101 and the second jaw 3102 are close to each other, such as when the first jaw 3101 and the second jaw 3102 are closed with the body tissue interposed therebetween, the operation member 3140 may push and raise the staples while moving toward a distal end 3101d of the first jaw 3101, so that stapling may be performed. At this point, one or more clamps 3146 and 3147 of the operation member 3140 may protrude to the outside of the first jaw 3101 and the second jaw 3102, allowing the operation member 3140 to move forward while applying pressure to an outer surface of the first jaw 3101 and the second jaw 3102, which facilitates the smooth progression of a stapling process. In an optional embodiment, the cartridge 3500 may include a case 3520 corresponding to the bottom, and the case 3520 is disposed in the first jaw 3101.

Meanwhile, the operation member 3140 may be used together with a wedge WDG. For example, the wedge WDG may be prepared separately from the operation member 3140 and then disposed adjacent to the operation member 3140 in the first jaw 3101. In addition, as another example, the operation member 3140 and the wedge WDG may be integrally formed. The wedge WDG may be disposed on at least one side of a body 3142 and may be formed to have a predetermined inclined surface. That is, the wedge WDG may be formed to be inclined by a certain degree with respect to the extension direction of the end tool 3100. In other words, the wedge WDG may be formed to have a greater height at a proximal end 3101p side of the first jaw 3101 than a distal end 3101d side of the first jaw 3101.

The wedge WDG may be formed to be sequentially in contact with withdrawal members 3535 (refer to FIG. 70) or a plurality of staples 3530 (refer to FIG. 70) disposed in the cartridge 3500, and may serve to sequentially push and raise the staples 3530.

The plurality of fixed pulleys 3120 may be disposed in the first jaw 3101 to be closer to the front of the cartridge 3500, i.e., to the distal end 3101d of the first jaw 3101, than the cartridge 3500. For example, a plurality of fixed pulleys 3120 may be disposed in a front space 3101c of the first jaw 3101, and details thereof will be described below.

In addition, the end tool 3100 of the surgical instrument of the present embodiment may include one or more members, such as joint members, that connect the jaw 3103 to the connector 3400. Further, in an optional embodiment, the end tool 3100 may include an end tool hub 3108 and a pitch hub 3107.

The end tool hub 3108 may be disposed to connect the end tool 3100 to the straight part 3401 of the connector 3400.

As an example, the end tool hub 3108 may have a pulley shaft JX4 corresponding thereto, and the pulley shaft JX4 may be a pitch rotation shaft. As a specific example, the end tool 3100 may perform a vertical rotational motion around the pulley shaft JX4 based on the drawing. In addition, one or more pulleys may be disposed to be adjacent to the pulley shaft JX4.

The end tool hub 3108 may be in the form of a bar extending from the center of a surface thereof that corresponds to the connector 3400, i.e., a bar extending from the center of a disk-shaped main region. The pulley shaft JX4 and a pulley shaft JX5 different from the pulley shaft JX4 may further correspond to a region of the bar.

The pitch hub 3107 is connected to the end tool hub 3108 and the jaw 3103. The pitch hub 3107 may be axially coupled to the end tool hub 3108 with respect to one pulley shaft, i.e., the pulley shaft JX4. The pitch hub 3107 may rotationally move around one pulley shaft, i.e., the pulley shaft JX4 while connected to the end tool hub 3108. That is, the end tool 3100 may perform a pitch motion as the pitch hub 3107 rotates around one pulley shaft, i.e., the pulley shaft JX4 with respect to the end tool hub 3108.

Further, the jaw 3103 of the end tool 3100 may be axially coupled to the pitch hub 3107 with respect to one pulley shaft, i.e., a pulley shaft JX1. The jaw 3103 may rotate around one pulley shaft, i.e., the pulley shaft JX1 while connected to the pitch hub 3107. That is, the jaw 3103 of the end tool 3100 may rotate around one pulley shaft, i.e., the pulley shaft JX1 with respect to the pitch hub 3107, thereby performing a yaw motion.

As a result, the yaw motion of the end tool 3100 includes a rotational motion of the jaw 3103 around one pulley shaft, i.e., the pulley shaft JX1 with respect to the pitch hub 3107, and the pitch motion of the end tool 3100 includes a rotational motion of the jaw 3103 coupled to the pitch hub 3107, which occurs as the pitch hub 3107 rotates around one pulley shaft, i.e., the pulley shaft JX4 together with the end tool hub 3108.

The pitch hub 3107 may include a first hub 3107a and a second hub 3107b.

The first hub 3107a of the pitch hub 3107 may be connected to the jaw 3103. As an example, the first hub 3107a may be elongated to connect to one region of the first jaw 3101, and specifically, may have two bars that are formed side by side to face each other and coupled to each other by placing one region of the first jaw 3101 therebetween.

The second hub 3107b of the pitch hub 3107 may be connected to the end tool hub 3108, for example, may have two bars that are formed side by side to face each other, and may be coupled to each other by placing one region of the end tool hub 3108 therebetween.

As described above, the pulley shaft JX5 is different from one pulley shaft, i.e., the pulley shaft JX4 may be disposed in the end tool hub 3108 to be spaced apart from the pulley shaft JX4 and closer to the connector 3400 (refer to FIG. 50) than the pulley shaft JX4. The pulley shaft JX4 and the pulley shaft JX5 may have axes in directions parallel to each other.

A pulley shaft JX2, which is different from the pulley shaft JX1, is disposed in the pitch hub 3107 in a direction adjacent to and parallel to the pulley shaft JX1. In addition, a pulley shaft JX3 and the pulley shaft JX4 may be formed in a direction different from (for example, intersecting or orthogonal to) the direction in which the pulley shaft JX1 and the pulley shaft JX2 are disposed, and may be sequentially disposed in a direction toward (or away from the operation member) the connector 1400.

The pulley shaft JX4 may be a pitch motion shaft of the end tool 3100, and the pulley shaft JX1 may be a yaw motion shaft.

The pulley shaft JX3 and the pulley shaft JX5 may be pitch auxiliary pulley shafts, and the pulley shaft JX2 may be a yaw auxiliary pulley shaft. One or more driving wires, such as a wire configured to transmit a driving force for a pitch motion or a yaw motion may have at least one region in contact with or wound around the pulley shafts JX1, JX2, JX3, JX4, and JX5.

The pulley shafts JX2, JX3, JX5 adjacent to the pulley shaft JX4, which is a pitch motion shaft, and the pulley shaft JX1, which is a yaw motion shaft, may control paths along which the driving wires are wound around the pulley shaft JX4 and the pulley shaft JX1 to secure the efficiency of the arrangement of the driving wires and stabilize the paths for transmitting forces through the driving wires.

In addition, at least one region of the forward-moving wire 3110 may be in contact with or wound around the pulley shafts JX1, JX2, JX3, JX4, and JX5.

A more detailed description of the arrangement of the pulley shafts JX1, JX2, JX3, JX4, and JX5 will be provided below.

One or more switching pulley shafts AX1 and AX2 may be disposed in the end tool 3100, and one or more pulleys corresponding to the switching pulley shafts AX1 and AX2 may be disposed.

For example, the switching pulley shafts AX1 and AX2 may be disposed in the jaw 3103, specifically, in the proximal end 3101p side of the first jaw 3101, and may be disposed closer to the distal end 3101d of the first jaw 3101 than at least the above-described pulley shafts JX1, JX2, JX3, JX4, and JX5.

The switching pulley shafts AX1 and AX2 may be shafts formed in parallel to each other, and may be disposed to have different backward and forward positions with respect to each other such that the switching pulley shaft AX1 and the switching pulley shaft AX2 are sequentially disposed with respect to the distal end 3101d of the first jaw 3101 and some regions of the switching pulley shaft AX1 and the switching pulley shaft AX2 are overlap each other.

The switching pulley shafts AX1 and AX2 may be regions where at least one region of the forward-moving wires 3110 is wound or comes into contact to organize and guide the path of the forward-moving wire 3110 before entering the pulley shafts JX1, JX2, JX3, JX4, and JX5. A more detailed description of the arrangement of the switching pulley shafts AX1 and AX2 will be provided below.

As shown in FIG. 55, the first forward-moving wire 3111 and the second forward-moving wire 3112 may be correspondingly wound around the first fixed pulley 3121 and the second fixed pulley 3122 in the first jaw 3101 to be redirected, and connected to the rear of the end tool 3100 via at least one region of each of the switching pulley shafts AX1 and AX2 and the pulley shafts JX1, JX2, JX3, JX4, and JX5. Furthermore, the first forward-moving wire 3111 and the second forward-moving wire 3112 may further extend to the operator 3200 (refer to FIG. 50) via the connector 3400 to be precisely controlled. Accordingly, precise motion control of the operation member 3140 may be easily implemented, and details thereof will be described below.

The jaw 3103 of the end tool 3100 will be described in more detail.

FIG. 56 is a perspective view schematically illustrating the second jaw of the end tool of FIG. 51.

The second jaw 3102 may be formed in an elongated bar shape as a whole, and for example, the second jaw 3102 may be formed in a rod shape to correspond to the first jaw 3101 in at least one region.

A proximal end 3102p of the second jaw 3102 may include a region that is coupled to the first jaw 3101. As an example, the proximal end 3102p is formed to be rotatable around the one shaft JX of the proximal end 3102p with respect to the first jaw 3101.

The second jaw 3102 may have various forms, and as a specific example, a plurality of anvil grooves may be formed in at least one region of a surface of the second jaw 3102 facing the first jaw 3101, and the anvil groove may have a shape corresponding to the shape of the staple 3530.

The anvil groove of the second jaw 3102 may serve as a support for allowing the staple 3530 to be bent when the operation member 3140 push and raise the staple 3530 during a staple motion.

The second jaw 3102 includes a guide groove 3102a. The guide groove 3102a may have a shape elongated in a longitudinal direction of the second jaw 3102.

The guide groove 3102a may be formed to guide the operation member 3140, and may be a groove passing through a region facing the operation member 3140. Through this, one region of the operation member 3140, such as at least one region of the body 3142 of the operation member 3140, or a first clamp 3146 connected thereto may pass through the guide groove 3102a to exit to the outside of the second jaw 3102. When the operation member 3140 moves forward, the first clamp 3146 may pass through the guide groove 3102a of the second jaw 3102 to be exposed to the outside of the second jaw 3102, and may come into contact with an upper surface of the second jaw 3102 or apply pressure thereto. As the operation member 3140 moves, the first clamp 3146 applies pressure on the upper surface of the second jaw 3102 and a second clamp 3147 to be described below applies pressure on a lower surface of the first jaw 3101 such that a gap between the second jaw 3102 and the first jaw 3101 decreases, allowing the second jaw 3102 to naturally remain in a closed state with respect to the first jaw 3101.

FIG. 57 is a perspective view schematically illustrating the first jaw of the end tool of FIG. 51. FIG. 58 is a plan view schematically illustrating the first jaw of the end tool of FIG. 51.

Referring to FIGS. 57 and 58 and the like, the first jaw 3101 is formed in an elongated bar shape as a whole, and a rotation shaft may be disposed in the proximal end such that the first jaw 3101 is rotationally movable, and such a rotation shaft may correspond to the rotation shaft JX formed in the second jaw 3102 described above. In addition, the cartridge 3500 (refer to FIG. 53) may be accommodated closer to the distal end 3101d side than the rotation shaft.

For example, the first jaw 3101 may be formed entirely in the form of a hollow box with one surface (upper surface) thereof is removed, such that a cartridge accommodation part 3101a capable of accommodating the cartridge 3500 may be formed inside the first jaw 3101. That is, the first jaw 3101 may be formed in a substantially “U” shape in cross section.

A guide groove 3101h may be formed in a bottom surface of the first jaw 3101, the bottom surface opposite to an upper open region formed by removing one surface. Specifically, the guide groove 3101h may be formed to guide a linear motion of the operation member 3140.

The guide groove 3101h may be formed to guide the operation member 3140, and may be a groove formed to pass through a region facing the operation member 3140. Through this, one region of the operation member 3140, such as at least one region of the body 3142 of the operation member 3140, or the second clamp 3147 connected thereto may pass through the guide groove 3101h to exit to the outside of the first jaw 3101. When the operation member 3140 moves forward, the second clamp 3147 may pass through the guide groove 3101h of the first jaw 3101 to be exposed to the outside of the first jaw 3101, and may come into contact with the lower surface of the first jaw 3101 or apply pressure thereto. As the operation member 3140 moves, the second clamp 3147 applies pressure on the lower surface of the first jaw 3101 and the first clamp 3146 applies pressure on the upper surface of the second jaw 3102 such that a gap between the second jaw 3102 and the first jaw 3101 decreases, allowing the second jaw 3102 to naturally remain in a closed state with respect to the first jaw 3101 (refer to FIGS. 20A, 20B, and 20C).

In an optional embodiment, the first jaw 3101 may include a window 3101b. After operating the operation member 3140 or using the end tool 3100, the second clamp 3147 of the operation member 3140 may be located corresponding to the window 3101b, and the coupled state of the first jaw 3101 and the operation member 3140 may be released.

The first jaw 3101 may include the front space 3101c located ahead of the cartridge accommodation part 3101a.

For example, the front space 3101c may be disposed closer to the distal end 3101d of the first jaw 3101 than the cartridge accommodation part 3101a. The plurality of fixed pulleys 3120 may be disposed in the front space 3101c, for example, the first fixed pulley 3121 and the second fixed pulley 3122 may be disposed in the front space 3101c (refer to, for example, FIGS. 51 to 55).

Two outer side surfaces of the front space 3101c include a first side surface 3101t1 and a second side surface 3101t2, and the first fixed pulley 3121 and the second fixed pulley 3122 may be disposed to correspond to the first side surface 3101t1 and the second side surface 3101t2, respectively.

Each of the first side surface 3101t1 and the second side surface 3101t2 may be formed to have an inclined shape. For example, the first side surface 3101t1 and the second side surface 3101t2 may be shaped such that a gap therebetween decreases as it moves downward, instead of being parallel to each other with the same gap. As a specific example, the gap between the first side surface 3101T1 and the second side surface 3101t2 may be formed to be smaller in a direction away from the second jaw 3102.

Since the first fixed pulley 3121 and the second fixed pulley 3122 are disposed to correspond to the first side surface 3101t1 and the second side surface 3101t2, respectively, the first fixed pulley 3121 and the second fixed pulley 3122 may be disposed to have an inclined shape such that a gap therebetween decreases in a direction away from the second jaw 3102 (refer to FIG. 51). The first fixed pulley 3121 and the second fixed pulley 3122 may be symmetrically shaped and may have the same size as each other.

In addition, a first path 3101w1 and a second path 3101w2 may be formed adjacent to the front space 3101c. For example, the first path 3101w1 and the second path 3101w2 may have the form of through holes formed in a barrier wall, and may be regions through which the first forward-moving wire 3111 and the second forward-moving wire 3112 pass, respectively.

In an optional embodiment, when a backward-moving wire (refer to, for example, FIG. 68) is disposed, the first jaw 3101 may include a rear side path 3101R corresponding to the backward-moving wire.

In addition, the first jaw 3101 may include a coupling region 3101z in a region adjacent to the proximal end 3101p. The coupling region 3101z is a region coupled to the pitch hub 3107 and may be in the form of a plate elongated to correspond to, for example, the first hub 3107a of the pitch hub 3107. The coupling region 3101z may be disposed and coupled between two bars formed on the first hub 3107a of the pitch hub 3107.

The operation member 3140 will now be described in detail.

FIG. 59 is a perspective view illustrating the operation member of the end tool of FIG. 51. FIG. 60 is a perspective view of the operation member of FIG. 59 viewed from another direction. FIG. 61 is a front view of the operation member of FIG. 59 viewed in one direction.

The operation member 3140 may include the body 3142, the first clamp 3146, and the second clamp 3147. Meanwhile, the operation member 3140 may be used together with the wedge WDG (refer to FIGS. 54 and 55). For example, the wedge WDG may be prepared separately from the operation member 3140 and then disposed adjacent to the operation member 3140 in the first jaw 3101. In addition, as another example, the operation member 3140 and the wedge WDG may be integrally formed. In the present specification, for convenience of description, the operation member 3140 and the wedge WDG will be described and illustrated in the drawings with the assumption that the operation member 3140 and the wedge WDG are prepared separately.

The wedge WDG may be disposed on at least one side of the body 3142 and may be formed to have a predetermined inclined surface. That is, the wedge WDG may be formed to be inclined by a certain degree with respect to the extension direction of the end tool 3100. In other words, the wedge WDG may be formed to have a greater height at a proximal end 3101p side of the first jaw 3101 than a distal end 3101d side of the first jaw 3101.

The wedge WDG may be formed to be sequentially in contact with the withdrawal members 3535 (refer to FIG. 70) or the plurality of staples 3530 (refer to FIG. 70) and may serve to sequentially push and raise the staples 3530.

The body 3142 may be in the form of an elongated column, such as a plate-shaped column. In addition, a blade region 3142a may be formed in one region of the body 3142, and an edge sharply formed to cut tissue may be formed in the blade region 3142a. The tissue disposed between the first jaw 3101 and the second jaw 3102 may be cut as at least a portion of the edge formed in the blade region 3142a of the body 3142 is withdrawn to the outside of the first jaw 3101 and the cartridge 3500.

The first clamp 3146 may be formed in one region of the body 3142, and the second clamp 3147 may be formed in another region different from the one region. For example, the body 3142 may be disposed between the first clamp 3146 and the second clamp 3147.

The first clamp 3146 and the second clamp 3147 may be formed to have a region with a width at least greater than that of the body 3142. Accordingly, the first clamp 3146 may be inserted into and pass through the guide groove 3102a formed in the second jaw 3102 in the longitudinal direction to be deposed or brought into contact with the upper surface of the second jaw 3102 and, at the same time, the second clamp 3147 may be inserted into and pass through the guide groove 3101h formed in the first jaw 3101 in the longitudinal direction to be deposed or brought into contact with the lower surface of the first jaw 3101, so that the first clamp 3146 and the second clamp 3147 may move. Thus, when the operation member 3140 moves, the first clamp 3146 and the second clamp 3147 may apply forces in directions that bring the second jaw 3102 and the first jaw 3101 closer to each other.

As a result, when the operation member 3140 moves from the proximal end 3101p of the first jaw 3101 toward the distal end 3101d of the first jaw 3101, a motion of decreasing a distance between the second jaw 3102 and the first jaw 3101, i.e., a closing motion of the jaw 3103, may be naturally implemented through the first clamp 3146 and the second clamp 3147.

The first clamp 3146 and the second clamp 3147 may be placed at different positions with respect to a forward-facing direction of the body 3142. For example, the second clamp 3147 may be located further forward than the first clamp 3146, for example, the second clamp 3147 may be located closer to the distal end 3101d of the first jaw 3101 than the first clamp 3146 when the operation member 3140 is disposed in the first jaw 3101. Accordingly, the operation member 3140 may move forward while the first jaw 3101 and second jaw 3102 are in the closed state, so that the first jaw 3101 and second jaw 3102 can be maintained with greater efficiency and stability while performing stapling.

A first connection region 3140p1 and a second connection region 3140p2 may be formed in one region of the body 3142, such as one region in a front side of the body 3142, specifically, a region of the body 3142 facing the distal end 3101d of the first jaw 3101.

The first connection region 3140p1 and the second connection region 3140p2 may be regions to which the first forward-moving wire 3111 and the second forward-moving wire 3112 are connected, respectively, and may each be in the form of a fixing groove such that respective one end portion region of the first forward-moving wire 3111 and the second forward-moving wire 3112 is accommodated or fixed thereto. When the first forward-moving wire 3111 and the second forward-moving wire 3112 are connected to the first connection region 3140p1 and the second connection region 3140p2 to pull the first forward-moving wire 3111 and the second forward-moving wire 3112, respectively, forces pulling the first forward-moving wire 3111 and the second forward-moving wire 3112 are transmitted to the operation member 3140 through the first connection region 3140p1 and the second connection region 3140p2 so that the operation member 3140 may move, i.e., move forward

In an optional embodiment, the first connection region 3140p1 and the second connection region 3140p2 may be formed on side portions 3143 of the operation member 3140, respectively. The side portions 3143 may be formed to protrude outward from both side surfaces of the body 3142, respectively. By forming the first connection region 3140p1 and the second connection region 3140p2 on the side portions 3143 formed to protrude from both sides of the body 3142, spaces for respectively connecting the first forward-moving wire 3111 and the second forward-moving wire 3112 to the first connection region 3140p1 and the second connection region 3140p2 can be easily secured.

Further, by forming the first connection region 3140p1 and the second connection region 3140p2 on the side portions 3143 formed on both sides of the body 3142, and connecting the first forward-moving wire 3111 and the second forward-moving wire 3112 to the first connection region 3140p1 and the second connection region 3140p2, the first forward-moving wire 3111 and the second forward-moving wire 3112 can be pulled from both sides of the body 3142, and also from symmetrical positions, so that the forward movement of the operation member 3140 can be precisely controlled.

In an optional embodiment, a connection region for the backward-moving wire may be formed in a rear side region of the body 3142.

FIG. 62 is a schematic perspective view illustrating a portion of the end tool of FIG. 51. FIG. 63 is a front view of FIG. 62 viewed in one direction.

Referring to FIGS. 62 and 63, the first fixed pulley 3121 and the second fixed pulley 3122 may be disposed to have an inclined shape without being disposed in parallel to each other. For example, as a specific example, based on the drawings, the first fixed pulley 3121 and the second fixed pulley 3122 may be disposed such that a gap therebetween decreases in a direction away from the second jaw 3102 (refer to FIG. 51).

Specifically, the first fixed pulley 3121 and the second fixed pulley 3122 may be disposed to face each other in the front space 3101c located further forward than the cartridge accommodation part 3101a of the first jaw 3101, and may be disposed symmetrically to each other as a specific example. In addition, the first fixed pulley 3121 and the second fixed pulley 3122 may be formed to have the same size.

Two outer side surfaces of the front space 3101c include the first side surface 3101t1 and the second side surface 3101t2, and the first fixed pulley 3121 and the second fixed pulley 3122 may be disposed to correspond to the first side surface 3101t1 and the second side surface 3101t2, respectively.

Each of the first side surface 3101t1 and the second side surface 3101t2 may be formed to have an inclined shape. For example, the first side surface 3101t1 and the second side surface 3101t2 may be shaped such that a gap therebetween decreases as it moves downward, instead of being parallel to each other with the same gap. As a specific example, the gap between the first side surface 3101t1 and the second side surface 3101t2 may be formed to be gradually smaller in a direction away from the second jaw 3102 (refer to FIG. 51). In addition, the first side surface 3101t1 and the second side surface 3101t2 may have shapes symmetrical to each other.

The first forward-moving wire 3111 and the second forward-moving wire 3112 may be correspondingly wound around the first fixed pulley 3121 and the second fixed pulley 3122, and the regions of the first forward-moving wire 3111 and the second forward-moving wire 3112 emerging from being wound around the lower sides of the first fixed pulley 3121 and the second fixed pulley 3122 may be directed to the first connection region 3140p1 and the second connection region 3140p2 of the operation member 3140 described above, respectively.

With such a shape, the characteristic of balanced arrangement of the first fixed pulley and the second fixed pulley 3121 and 3122 and the first forward-moving wire and the second forward-moving wire 3111 and 3112 with respect to the moving direction of the operation member 3140 may be improved, and for example, the symmetrical shape may be easily implemented. In addition, shaking or rotational moment generated when pulling the first forward-moving wire 3111 and the second forward-moving wire 3112 may be reduced, so that the end tool 3100 may be reduced or prevented from shaking.

In addition, the end tool 3100 may be shaped such that a width of, for example, one side of the first jaw 3101, which is one side of the jaw 3103, specifically, a width of a main region of the lower side is reduced, so that the end tool 3100 can be implemented in a compact structure as a whole.

Further, the first fixed pulley 3121 and the second fixed pulley 3122 may be disposed to have an inclined shape without being disposed in parallel to each other, which allows the two forward-moving wires 3111 and 3112 to emerge from being wound around the lower sides of the first fixed pulley 3121 and the second fixed pulley 3122 and be connected to the operation member 3140, i.e., disposed in a lower side region of the cartridge 3500 after passing through the first path 3101w1 and the second path 3101w2. Another regions of the two forward-moving wires 3111 and 3112 may emerge from being wound around the upper sides of the first fixed pulley 3121 and the second fixed pulley 3122 and may be respectively disposed on both sides of the cartridge 3500, i.e., in spaces between the cartridge 3500 and both side surfaces of the first jaw 3101.

By arranging the two forward-moving wires 3111 and 3112 in this manner, the forward-moving wires 3111 and 3112 are avoided from being fixed to the center line of the operation member 3140, so that the operation member 3140 can be implemented in a compact form while maintaining durability.

In addition, by arranging the two forward-moving wires 3111 and 3112 correspondingly to the first fixed pulley 3121 and the second fixed pulley 3122 formed in an inclined shape, such as a symmetrical shape, maximum tensions applied to the two forward-moving wires 3111 and 3112 can be equalized or made almost similar to each other, this may enhance the fatigue life of each wire.

In addition, one regions of the two forward-moving wires 3111 and 3112 can be controlled to symmetrically pass through the lower side of the cartridge 3500, and another regions thereof can be controlled to symmetrically pass through both sides of the cartridge 3500, and accordingly, unwanted moment or rotational forces generated when pulling the two forward-moving wires 3111 and 3112 may be reduced or prevented from causing unintended movement or shaking of the end tool 3100 or the surgical instrument 3000 including the same.

Meanwhile, referring to FIG. 63, the first fixed pulley 3121 and the second fixed pulley 3122 may be disposed in a slanted shape, i.e., an inclined shape, without being disposed in parallel to each other.

For example, each of the first fixed pulley 3121 and the second fixed pulley 3122 may be disposed to be inclined at an angle of 35 degrees (deg) to 51 degrees (deg) relative to a vertical line (a line parallel to a z-axis) based on FIG. 63.

Further, in other words, a central axis of the first fixed pulley 3121 and a central axis of the second fixed pulley 3122 may be inclined at an angle without being parallel to each other, and for example, the central axis of the first fixed pulley 3121 and the central axis of the second fixed pulley 3122 may be disposed to be inclined at an angle of 78 degrees to 110 degrees.

Further, in other words, as shown in FIG. 63, the first fixed pulley 3121 and the second fixed pulley 3122 may be shaped to be inclined, forming an adjacent angle DG.

The adjacent angle DG may range from 70 degrees (deg) to 102 degrees (deg).

When the adjacent angle DG is less than 70 degrees or more than 102 degrees, a diameter of each of the first fixed pulley 3121 and the second fixed pulley 3122 accommodated in a predetermined space of the first jaw 3101 becomes smaller, which causes the stress applied on the forward-moving wires 3111 and 3112 to be increased, and the maximum allowable tension and durability of the forward-moving wires 3111 and 3112 to be reduced, and thus, the adjacent angle DG may range from 70 degrees (deg) to 102 degrees (deg), in a specific example, the adjacent angle DG may be 86 degrees (deg). Meanwhile, the adjacent angle DG is applicable to embodiments to be described below without change.

The arrangement relationship of the operation member 3140, the plurality of forward-moving wires 3110, and the plurality of fixed pulleys 3120 will now be further described.

FIG. 64 is a schematic plan view for describing the operation member, the fixed pulley, and the forward-moving wire of the end tool of FIG. 51. FIG. 65 is a schematic perspective view for describing the operation member, the fixed pulley, and the forward-moving wire of the end tool of FIG. 51.

As described above, the first fixed pulley 3121 and the second fixed pulley 3122 are disposed in the front space 3101c of the first jaw 3101, and each of the first fixed pulley 3121 and the second fixed pulley 3122 may be fixed to the first jaw 3101 and immobile, or fixed so as to be rotatable about one shaft.

The first forward-moving wire 3111 and the second forward-moving wire 3112 may be wound around outer circumferential surfaces of the first fixed pulley 3121 and the second fixed pulley 3122, respectively, and a groove may be formed in each of the outer circumferential surfaces of the first fixed pulley 3121 and the second fixed pulley 3122.

The first fixed pulley 3121 and the second fixed pulley 3122 may be disposed closer to the distal end 3101d of the first jaw 3101 at least than the operation member 3140.

The first forward-moving wire 3111 may extend to have a length in the longitudinal direction of the first jaw 3101, and one end portion region thereof may extend to the proximal end 3101p of the first jaw 3101, and pass through the switching pulley shafts AX1 and AX2 or switching pulleys coupled thereto and the pulley shafts JX1, JX2, JX3, JX4, and JX5 or pulleys coupled thereto to be connected to the inside of the driving part, such as the operator 3200 (refer to FIG. 51), so that the first forward-moving wire 3111 can be pulled through manipulation of the operator 3200.

Another end portion of the first forward-moving wire 3111 may extend in a direction toward the distal end 3101d of the first jaw 3101 along the longitudinal direction of the first jaw 3101, emerge from the lower side of the first fixed pulley 3121 after being wound around the upper side of the first fixed pulley 3121 while coming into contact therewith, and extend in a direction toward proximal end 3101p of the first jaw 3101 to be connected and fixed to the first connection region 3140p1 of the operation member 3140.

In an optional embodiment, among the regions of the first forward-moving wire 3111, the region extending to the distal end 3101d of the first jaw 3101 and being directed to the upper side of the first fixed pulley 3121 may be parallel to the region emerging from the lower side of the first fixed pulley 3121, extending toward the proximal end 3101p of the first jaw 3101, and being directed toward the first connection region 3140p1 of the operation member 3140.

Accordingly, a pulling force can be effectively transmitted to the operation member 3140 when pulling the first forward-moving wire 3111 toward the proximal end 3101p. In addition, as an example, when pulling the first forward-moving wire 3111 toward the proximal end 3101p, a ratio of the pulling force to the corresponding forward-moving distance of the operation member 3140 may be precisely controlled at a predetermined ratio. As a specific example, when pulling the first forward-moving wire 3111 toward the proximal end 3101p, the ratio of the pulling force to the forward-moving distance of the operation member 3140 may be easily controlled to a value equal to, or substantially equal or similar to 1:1.

The second forward-moving wire 3112 may extend to have a length in the longitudinal direction of the first jaw 3101, and one end portion region thereof may extend to the proximal end 3101p of the first jaw 3101, and pass through the switching pulley shafts AX1 and AX2 or switching pulleys coupled thereto and the pulley shafts JX1, JX2, JX3, JX4, and JX5 or pulleys coupled thereto to be connected to the inside of the driving part, such as the operator 3200 (refer to FIG. 51), so that the second forward-moving wire 3112 can be pulled through manipulation of the operator 3200.

Another end portion of the second forward-moving wire 3112 may extend in a direction toward the distal end 3101d of the first jaw 3101 along the longitudinal direction of the first jaw 3101, emerge from the lower side of the second fixed pulley 3122 after being wound around the upper side of the second fixed pulley 3122 while coming into contact therewith, and extend in a direction toward proximal end 3101p of the first jaw 3101 to be connected and fixed to the second connection region 3140p2 of the operation member 3140.

In an optional embodiment, among the regions of the second forward-moving wire 3112, the region extending to the distal end 3101d of the first jaw 3101 and being directed to the upper side of the second fixed pulley 3122 may be parallel to the region emerging from the lower side of the second fixed pulley 3122, extending toward the proximal end 3101p of the first jaw 3101, and being directed toward the second connection region 3140p2 of the operation member 3140.

Accordingly, a pulling force can be effectively transmitted to the operation member 3140 when pulling the second forward-moving wire 3112 toward the proximal end 3101p.

As shown in FIGS. 64 and 65, when the first forward-moving wire 3111 and the second forward-moving wire 3112 are pulled in a first direction D1, one region of each of the first forward-moving wire 3111 and the second forward-moving wire 3112 may move in the first direction D1, and accordingly, the region of the forward-moving wire 3110 emerging from the lower sides of the first fixed pulley 3121 and the second fixed pulley 3122 after being wound around the upper sides thereof may move in a second direction D2, which is the opposite direction of the first direction D1. Accordingly, forces of the first forward-moving wire 3111 and the second forward-moving wire 3112 may be transmitted to the first connection region 3140p1 and the second connection region 3140p2 connected to the first forward-moving wire 3111 and the second forward-moving wire 3112, and the forces cause the operation member 3140 also move in a direction K1, which is the same direction as the second direction D2, i.e., move forward.

FIGS. 66 and 67A, 67B, and 67C are schematic views for describing an operation of the operation member of the end tool of FIG. 51.

Referring to FIGS. 66 and 67A, 67B, and 67C, for convenience of description, the first jaw 3101 is excluded, and the first forward-moving wire 3111, the second forward-moving wire 3112, the first fixed pulley 3121, the second fixed pulley 3122, and the operation member 3140 are illustrated.

Based on FIG. 66, the operation member 3140 may move in a leftward direction, i.e., move forward toward the distal end 3101d, and this forward movement is illustrated sequentially in FIGS. 67A, 67B, and 67C.

As shown in FIG. 67A, when the first forward-moving wire 3111 and the second forward-moving wire 3112 are pulled in the first direction D1, one region of each of the first forward-moving wire 3111 and the second forward-moving wire 3112 is pulled in the first direction D1, and thus the regions of the first forward-moving wire 3111 and the second forward-moving wire 3112 emerging from the lower sides of the first fixed pulley 3121 and the second fixed pulley 3122 after being wound around the upper sides thereof move in the second direction D2, which is the opposite direction of the first direction D1. Accordingly, forces of the first forward-moving wire 3111 and the second forward-moving wire 3112 are transmitted respectively to the first connection region 3140p1 and the second connection region 3140p2 respectively connected to the first forward-moving wire 3111 and the second forward-moving wire 3112, and the forces cause the operation member 3140 to move in the direction K1, which is the same direction as the second direction D2, i.e., move forward, thereby positioning the operation member 3140 in an advanced position shown in FIG. 67B.

Thereafter, as shown in FIG. 67B, when the first forward-moving wire 3111 and the second forward-moving wire 3112 are pulled further in the first direction D1, one region of the first forward-moving wire 3111 and the second forward-moving wire 3112 is pulled further in the first direction D1, and thus the regions of the first forward-moving wire 3111 and the second forward-moving wire 3112 emerging from the lower sides of the first fixed pulley 3121 and the second fixed pulley 3122 after being wound around the upper sides thereof move further in the second direction D2, which is the opposite direction of the first direction D1. Accordingly, forces of the first forward-moving wire 3111 and the second forward-moving wire 3112 are transmitted respectively to the first connection region 3140p1 and the second connection region 3140p2 connected to the first forward-moving wire 3111 and the second forward-moving wire 3112, and the forces cause the operation member 3140 to move further in the direction K1, which is the same direction as the second direction D2, i.e., move forward to a further advanced position than in FIG. 67B, thereby positioning the operation member 3140 in the further advanced position shown in FIG. 67C.

Although not shown in the drawings, it will be appreciated of course that the form corresponding to the side view illustrating the operation of the operation member of FIGS. 20A, 20B, and 20C may be applied to the end tool 3100 of the present embodiment either as is or with appropriate modifications.

FIGS. 68 and 69A, 69B, and 69C are views for describing an optional embodiment in which a backward-moving wire is added to the end tool of FIG. 51.

Referring to FIGS. 68 and 69A, 69B, and 69C, the end tool of the present embodiment may further include a backward-moving wire BRW.

For example, the structure of FIG. 68 may be the structure of FIG. 66 with the addition of the backward-moving wire BRW.

Referring to FIGS. 68 and 69A, 69B, and 69C, for convenience of description, the first jaw 3101 is excluded, and the first forward-moving wire 3111, the second forward-moving wire 3112, the first fixed pulley 3121, the second fixed pulley 3122, the operation member 3140, and the backward-moving wire BRW are illustrated.

Based on FIG. 68, the operation member 3140 may move in a rightward direction, i.e., move backward toward the proximal end 3101p, and this backward movement is illustrated sequentially in FIGS. 69A, 69B, and 69C.

The backward-moving wire BRW may be connected to one region of the operation member 3140 and may be connected, for example, to a rear side of the operation member, specifically, to a region of the blade region 3142a, which is opposite to the region in which the edge of the blade region 3142a is formed, among regions of the body 3142.

A driving part or driving transmission part (e.g., a wire, a pulley, or the like) capable of pulling the backward-moving wire BRW may be connected to the backward-moving wire BRW, and the backward-moving wire BRW may be operated according to manual or automatic manipulation. For example, the backward-moving wire BRW may be pulled by the operator 3200 (refer to FIG. 50).

By pulling the backward-moving wire BRW. The operation member 3140 may move backward.

For example, as shown in FIG. 69A, the backward-moving wire BRW is pulled in a reverse direction B1 in a state in which the operation member 3140 is located adjacent to the distal end 3101d of the first jaw 3101, the operation member 3140 connected to the backward-moving wire BRW moves backward in a direction K2, which is the same direction as the reverse direction B1.

At this point, the first forward-moving wire 3111 and the second forward-moving wire 3112 may be in a state in which a pulling force is not applied.

When the operation member 3140 moves backward (in the direction K2), the region of the first forward-moving wire 3111 and the second forward-moving wire 3112 connected to the first connection region 3140p1 and the second connection region 3140p2 of the operation member 3140 may move in the first direction D1, which is the same direction as the direction K2, and the regions of the first forward-moving wire 3111 and the second forward-moving wire 3112, which are wound around the lower sides of the first fixed pulley 3121 and the second fixed pulley 3122 and disposed on the upper sides thereof, may move in the second direction D2, which is the opposite direction of the direction K2. Accordingly, the operation member 3140 is in a position as shown in FIG. 69B, having moved backward to be closer to the proximal end 3101p compared to its position in FIG. 69A.

Thereafter, as shown in FIG. 69B, when the backward-moving wire BRW is pulled further in the reverse direction B1, the operation member 3140 connected to the backward-moving wire BRW moves backward in the direction K2, which is the same direction as the reverse direction B1. At this point, the first forward-moving wire 3111 and the second forward-moving wire 3112 may be in a state in which a pulling force is not applied. The regions of the first forward-moving wire 3111 and the second forward-moving wire 3112 connected to the first connection region 3140p1 and the second connection region 3140p2 of the operation member 3140 move in the first direction D1, which is the same direction as the direction K2, and the regions of the first forward-moving wire 3111 and the second forward-moving wire 3112, which are wound around the lower sides of the first fixed pulley 3121 and the second fixed pulley 3122 and disposed on the upper sides thereof, may move in the second direction D2, which is the opposite direction of the direction K2. Accordingly, the operation member 3140 is in a position as shown in FIG. 69C, having moved backward to be closer to the proximal end 3101p compared to its position in FIG. 69B.

Further, although not shown in the drawings, it is of course possible that the configuration of FIG. 27 described above may be optionally applied to the end tool 3100 of the present embodiment.

The cartridge 3500 accommodated in the end tool 3100 of FIG. 51 and a stapling motion will now be described in more detail.

FIG. 70 is a perspective view illustrating the first jaw and the cartridge of the surgical instrument of FIG. 50.

Referring to FIGS. 50, 51, 52, 53, and 70 and the like, the cartridge 3500 may be disposed in the first jaw 3101, and for example, the cartridge 3500 may be disposed by being coupled to the cartridge accommodation part 3101a of the first jaw 3101. For example, the cartridge 3500 may be integrally formed with the first jaw 3101 while the operation member 3140 is disposed in the first jaw 3101. In addition, in an optional embodiment, the cartridge 3500 may be formed to be mountable to and dismountable from the first jaw 3101.

The cartridge 3500 includes a plurality of staples 3530 therein to perform suturing of tissue, and performs cutting through the operation member 3140. Here, the cartridge 3500 may include a cover 3510, the staples 3530, and the withdrawal members 3535.

The cover 3510 may be formed to cover an upper portion of the cartridge accommodation part 3101a of the first jaw 3101. Staple holes 3510s through which the plurality of staples 3530 may be ejected to the outside may be formed in the cover 3510. As the staples 3530, which are accommodated inside the cartridge accommodation part 3101a before a stapling operation, are pushed and raised upward by the operation member 3140 during a stapling motion, and pass through the staple holes 3510s of the cover 3510 to be withdrawn to the outside of the cartridge 3500, stapling may be performed.

Meanwhile, a slit 3510w may be formed in the cover 3510 along a longitudinal direction of the cover 3510. The blade region 3142a of the body 3142 of the operation member 3140 may protrude out of the cartridge 3500 through the slit 3510w. As the blade of the body 3142 of the operation member 3140 passes along the slit 3510w, staple-completed tissue may be cut.

In an optional embodiment, the cartridge 3500 may include a case 3520, and the cartridge 3500 may be disposed in the case 3520 after the case 3520 is disposed in the cartridge accommodation part 3101a of the first jaw 3101.

The plurality of staples 3530 may be disposed inside the cartridge accommodation part 3101a of the first jaw 3101. As the operation member 3140 linearly moves in one direction, the plurality of staples 3530 are sequentially pushed and raised from the inside of the cartridge accommodation part 3101a of the first jaw 3101 to the outside, thereby performing suturing, that is, stapling. Here, the staples 3530 may include a material that is durable and does not have an abnormal effect on the human body, such as titanium, stainless steel, or the like.

Meanwhile, the withdrawal members 3535 may be further disposed between the cartridge accommodation part 3101a of the first jaw 3101 and the staples 3530. In other words, it may be said that the staple 3530 is disposed above the withdrawal member 3535. In this case, the operation member 3140 linearly moves in one direction to push and raise the withdrawal member 3535, and the withdrawal member 3535 may push and raise the staple 3530.

As such, the operation member 3140 may be described as pushing and raising the staples 3530 in both the case in which the operation member 3140 directly pushes and raises the staples 3530 and the case in which the operation member 3140 pushes and raises the withdrawal members 3535 and the withdrawal members 3535 pushes and raises the staples 3530 (i.e., the operation member 3140 indirectly pushes and raises the staples 3530).

As described above, the operation member 3140 may be disposed inside the cartridge accommodation part 3101a of the first jaw 3101. In addition, the operation member 3140 may include the wedge WDG or may be used in conjunction with wedge WDG, and when the operation member 3140 moves, the wedge WDG may move together therewith so that the wedge WDG may directly push and raise the staple 3530, or the wedge WDG may push and raise the withdrawal member 3535 to push and raise the staple 3530.

As described above, the movement of the first forward-moving wire 3111 and the second forward-moving wire 3112, i.e., the pulling of the first forward-moving wire 3111 and the second forward-moving wire 3112 allows the operation member 3140 connected thereto to move forward toward the distal end 3101d of the first jaw 3101.

The forward movement of the operation member 3140 may cause the wedge WDG to push and raise the withdrawal member 3535, which may also cause the staple 3530 to rise, and at the same time, cutting using the blade region 3142a of the operation member 3140 may be performed. In addition, in an optional embodiment, in the case of the end tool in which the backward-moving wire BRW is connected to the operation member 3140, the backward-moving wire BRW may be pulled to cause the operation member 3140 to move toward the proximal end 3101p of the first jaw 3101.

FIGS. 71 and 72 are views for describing a switching pulley, a yaw pulley, and a pitch pulley of the end tool of the surgical instrument of FIG. 50.

As described above, the end tool 3100 may be connected to the connector 3400, and the end tool 3100 may rotationally move around one shaft and also rotationally move around another shaft with respect to the connector 3400.

For example, the end tool 3100 may perform a pitch motion, i.e., a vertical rotational motion based on FIGS. 51 and 70, and the end tool 3100 may perform a yaw motion, i.e., a horizontal rotational motion based on FIGS. 51 and 70. A rotation shaft of the pitch motion and a rotation shaft of the yaw motion may be located in directions that may intersect or be perpendicular to each other.

As an example, the end tool 3100 may include one or more members, such as joint members, that connect the jaw 3103 to the connector 3400, and may include the end tool hub 3108 and the pitch hub 3107.

The end tool hub 3108 may be disposed to connect the end tool 3100 to the straight part 3401 of the connector 3400. As an example, the end tool hub 3108 may have the pulley shaft JX4 corresponding thereto, and the pulley shaft JX4 may be a rotation shaft of the pitch motion. As a specific example, the end tool 3100 may rotationally move around the pulley shaft JX4, the pitch hub 3107 may rotationally move around the pulley shaft JX4, and the jaw 3103 may be connected to the pitch hub 3107 to perform a rotational motion, i.e., a pitch motion, around the pulley shaft JX4 integrally with the pitch hub 3107.

The pitch hub 3107 is connected to the end tool hub 3108 and the jaw 3103, and may rotationally move around the pulley shaft JX4 by being axially coupled to the end tool hub 3108 by the pulley shaft JX4. Further, the jaw 3103 may be axially coupled to the pitch hub 3107 with respect to one pulley shaft, i.e., the pulley shaft JX1. The jaw 3103 may perform a rotational motion, i.e., a yaw motion, around one pulley shaft, i.e., the pulley shaft JX1 while connected to the pitch hub 3107.

An auxiliary pulley shaft may be additionally disposed together with these rotation shafts, i.e., the pulley shafts for joint motion of the end tool 3100, such as the pulley shaft JX4 for pitch motion and the pulley shaft JX1 for yaw motion.

For example, the pulley shaft JX2 which is different from the pulley shaft JX1 is disposed in the pitch hub 3107 to be adjacent to and parallel to the pulley shaft JX1. The pulley shaft JX2 may have an axis oriented parallel to the pulley shaft JX1, and may be disposed further away from the operation member 3140 than the pulley shaft JX1, i.e., closer to the connector 3400.

In addition, the pulley shaft JX3 may be disposed adjacent to the pulley shaft JX4. In addition, the pulley shaft JX5 may be further disposed.

For example, the pulley shaft JX3 and the pulley shaft JX5 may be disposed on both sides of the pulley shaft JX4 interposed therebetween, and the pulley shaft JX3 and the pulley shaft JX5 may have axes oriented parallel to the pulley shaft JX4.

As a specific example, the pulley shaft JX3 may be disposed between the pulley shaft JX2 and the pulley shaft JX4, and may have an axis intersecting or orthogonal to the pulley shaft JX2 and the pulley shaft JX1. The pulley shaft JX5 may be disposed further away from the operation member 3140 than the pulley shaft JX4, i.e., closer to the connector 3400.

One or more switching pulley shafts AX1 and AX2, i.e., a first switching pulley shaft AX1 and a second switching pulley shaft AX2, may be disposed closer to the operation member 3140 than the pulley shafts JX1, JX2, JX3, JX4, and JX5

The first switching pulley shaft AX1 and the second switching pulley shaft AX2 may be shafts formed parallel to each other. The first switching pulley shaft AX1 and the second switching pulley shaft AX2 may be sequentially disposed in a direction toward the distal end 3101d of the first jaw 3101 so as to be offset from each other based on a width direction of the first jaw 3101, and may be disposed with some regions overlapping.

One or more pulleys may be disposed on the pulley shafts JX1, JX2, JX3, JX4, and JX5 and the switching pulley shafts AX1 and AX2.

When description is given in the order from the proximal end of the first jaw 3101 towards the connector 3400, one or more switching pulleys AXP1 corresponding to the first switching pulley shaft AX1 and one or more switching pulleys AXP2 corresponding to the second switching pulley shaft AX2 are disposed.

The one or more switching pulleys AXP1 corresponding to the first switching pulley shaft AX1, and the one or more switching pulleys AXP2 corresponding to the second switching pulley shaft AX2 may all be disposed in a place overlapping the first jaw 3101, for example, may be disposed in one region of the coupling region 3101z of the first jaw 3101 so as not to overlap the pitch hub 3107.

The first forward-moving wire 3111 and the second forward-moving wire 3112 may come into contact with the switching pulleys AXP1 and the switching pulley AXP2, respectively, in at least one region, and thus may be guided in path.

For example, the first forward-moving wire 3111 may enter the switching pulleys AXP1 from the outside, wind inward, continue winding into one region of the switching pulleys AXP2 adjacent to the switching pulleys AXP1, and then exit from the switching pulleys AXP2.

The second forward-moving wire 3112 may come into contact with the switching pulleys AXP2 in at least one region, and may be guided in path. The first forward-moving wire 3111 and the second forward-moving wire 3112, which are collected in the switching pulley AXP2, may be wound together around one or more pulleys JXP1 corresponding to the pulley shaft JX1, which will be described below, in the same direction.

Accordingly, the first forward-moving wire 3111 and the second forward-moving wire 3112 can be moved in one direction, and can be organized on one side of the shaft and pulley for each joint motion, such as the pulley shaft and pulleys for pitch motion and the pulley shaft and pulleys for yaw motion, rather than on both sides of the shaft and pulley for each joint motion, thus facilitating precise implementation of simultaneous and straightforward control over the first forward-moving wire 3111 and the second forward-moving wire 3112.

For example, when the end tool 3100 performs yaw and pitch motions, the wires wound around the yaw pulley (for yaw motion) or the pitch pulley (for pitch motion) may be wound or unwound further to one side, whereas the first forward-moving wire 3111 and the second forward-moving wire 3112 of the present embodiment can be gathered and organized on one side, i.e., wound in the same direction on the yaw pulley and the pitch pulley, and thus may be wound or unwound the same distance. Accordingly, the two wires can be easily controlled together by a single driving unit (e.g., an actuator) since the first forward-moving wire 3111 and the second forward-moving wire 3112 need only be wound or unwound by the same distance in order to make yaw and/or pitch manipulation to prevent the operation member 3140 from moving.

In an optional embodiment, the first forward-moving wire 3111 and the second forward-moving wire 3112 may be wound in different directions around the yaw pulley and the pitch pulley, respectively. When the first forward-moving wire 3111 or the second forward-moving wire 3112 is pulled, an undesired rotational force may be generated in the end tool 3100, which may occur because each of the first forward-moving wire 3111 and the second forward-moving wire 3112 is offset by a radius of the yaw pulley or the pitch pulley, causing asymmetrical forces to be applied to the end tool 3100. At this point, when the first forward-moving wire 3111 and the second forward-moving wire 3112 are wound in opposite directions around the yaw pulley and the pitch pulley, respectively, the rotational forces in the end tool 3100 may be canceled through application of symmetrical forces, thereby reducing a change in position/posture of the end tool 3100 during a stapling process. In this case, the first forward-moving wire 3111 and the second forward-moving wire 3112 may be wound around one side and unwound around another side, and thus a compensating member may be further disposed to compensate for this. As an example, the first forward-moving wire 3111 and the second forward-moving wire 3112 may be formed into a single loop by placing a common pulley in one region of the end tool 3100, or the connector 3400 or the operator 3200 adjacent thereto. As another example, the first forward-moving wire 3111 and the second forward-moving wire 3112 may each be manipulated by a separate driving unit (e.g., an actuator).

In order to facilitate the path guidance for the first forward-moving wire 3111 and the second forward-moving wire 3112, the switching pulley AXP1 and the switching pulley AXP2 may have structures symmetrical to each other with respect to an extension line of the operation member 3140, that is, may have structures offset by the same distance with respect to the extension line of the operation member 3140. This allows the size of each of the switching pulley AXP1 and the switching pulley AXP2 to be increased, thereby improving the efficiency and stability of the path guidance for the first forward-moving wire 3111 and the second forward-moving wire 3112.

One or more pulleys JXP1 are disposed to correspond to the pulley shaft JX1, and one or more pulleys JXP2 corresponding to the pulley shaft JX2 are disposed adjacent to the one or more pulleys JXP1. The pulley JXP1 and the pulley JXP2 may have axes parallel to each other.

For example, the pulley JXP1 and the pulley JXP2 are disposed in the first hub 3107a (refer to FIG. 54) of the pitch hub 3107. One or more pulleys JXP2 guide the paths along which the wires, which are disposed to correspond to one or more pulleys JXP1, are driven, ensuring that the wires have a clear path to the pulley shaft JX4, or more closely, to the pulley shaft JX3 and a pulley JXP3 corresponding thereto.

Further, one or more pulleys JXP3 are disposed to correspond to the pulley shaft JX3, and one or more pulleys JXP4 corresponding to the pulley shaft JX4 are disposed adjacent to the one or more pulleys JXP4. For example, the pulley JXP3 and the pulley JXP4 are disposed in the second hub 3107b (refer to FIG. 54) of the pitch hub 3107. In addition, one or more pulleys JXP5 may be disposed to correspond to the pulley shaft JX5. The pulley JXP3, the pulley JXP4, and the pulley JXP5 may have axes that are parallel to each other and intersecting or orthogonal to the axes of the pulley JXP1 and the pulley JXP2.

Meanwhile, by precisely controlling the paths of the first forward-moving wire 3111 and the second forward-moving wire 3112 as shown in FIGS. 71 and 72, the drive efficiency and control characteristics of the operation member 3140 can be maximized through the first forward-moving wire 3111 and the second forward-moving wire 3112.

As described above, the first switching pulley AXP1 and the second switching pulleys AXP2 are disposed further forward than the pulley shaft JX4 and the pulley shaft JX1, which are respectively for two joint motions such as a pitch motion and a yaw motion of the end tool 3100, i.e., disposed closer to the operation member 3140 than the above pulleys.

As a specific example, the first switching pulley AXP1 and the second switching pulleys AXP2 are disposed further forward than the pulley JXP4 corresponding to the pulley shaft JX4 for a pitch motion, the pulley JXP1 corresponding to the pulley shaft JX1 for a yaw motion, the pulley JXP3 and the pulley JXP5 corresponding to pitch auxiliary pulleys, and the pulley JXP2 that is a yaw auxiliary pulley, i.e., disposed closer to the operation member 3140 than the above pulleys.

As a result, the first switching pulley AXP1 and the second switching pulleys AXP2 are disposed further forward than the pulley JXP1, the pulley JXP2, the pulley JXP3, the pulley JXP4, and the pulley JXP5, i.e., disposed closer to the operation member 3140 than the above pulleys.

Accordingly, the first forward-moving wire 3111 may enter the switching pulleys AXP1 from the outside, wind inward, continue winding into one region of the switching pulleys AXP2 adjacent to the switching pulleys AXP1, and then exit from the switching pulleys AXP2. In addition, the first forward-moving wire 3111 and the second forward-moving wire 3112 may be gathered together on one side (e.g., an outer side) of the switching pulley AXP2.

In addition, the first forward-moving wire 3111 and the second forward-moving wire 3112, which are gathered together on the outer side of the switching pulley AXP2, may be directed to the connector 3400 after being simultaneously rerouted at an outer side of the pulley JXP1 corresponding to the pulley shaft JX1, which is a yaw pulley shaft, being wound around the pulley JXP2 corresponding to the pulley shaft JX2, which is a yaw auxiliary pulley shaft, to be rerouted, being controlled in path height by the pulley JXP3 corresponding to the pulley shaft JX3, being stably placed on a lower side of the pulley JXP4 corresponding to the pulley shaft JX4, which is a pitch shaft, and passing through the pulley JXP5.

That is, by first gathering the first forward-moving wire 3111 and the second forward-moving wire 3112 together through the switching pulley AXP1 and the switching pulley AXP2, the paths of the first forward-moving wire 3111 and the second forward-moving wire 3112 can be easily guided by simultaneously corresponding to the rotation shafts and the pulleys and their auxiliary pulleys for the joint motion of the end tool 3100, so that the accuracy and stability of the forward movement of the operation member 3140 can be improved.

In addition, by controlling heights of the paths of the first forward-moving wire 3111 and the second forward-moving wire 3112, which are gathered together, using the pulley JXP3 prior to directing to the pulley JXP4 corresponding to the pulley shaft JX4, which is a pitch shaft, the first forward-moving wire 3111 and the second forward-moving wire 3112 can be stably wound around the pulley JXP4 corresponding to the pulley shaft JX4, which is a pitch shaft, and the freedom of size, design and arrangement of the pulley JXP4 can be improved.

In an optional embodiment, the backward-moving wire BRW may be further disposed as described above, in which case the backward-moving wire BRW may pass between the switching pulley AXP1 and the switching pulley AXP2, or pass in contact with a common region of the switching pulley AXP1 and the switching pulley AXP2, and then pass while corresponding to the other pulleys JXP1, JXP2, JXP3, JXP4, and JXP5.

For example, the backward-moving wire BRW may correspond to the switching pulley disposed on the shaft parallel to or the same shaft as at least one of the switching pulley shafts AX1 and AX2, and specifically, may pass to correspond to the one or more pulleys disposed on the switching pulley shafts AX1 and AX2 and opposite to the switching pulley AXP1 or the switching pulley AXP2.

Meanwhile, the backward-moving wire BRW may be wound around the pulleys JXP1 and JXP2 in a direction opposite to the direction in which the first forward-moving wire 3111 and the second forward-moving wire 3112 are wound. For example, when the first forward-moving wire 3111 and the second forward-moving wire 3112 are wound around a rear side of the pulley JXP1 and wound around a front side of the pulley JXP2, the backward-moving wire BRW may be wound around a front side of the pulley JXP1 and wound around a rear side of the pulley JXP2. This allows the first forward-moving wire 3111 and the second forward-moving wire 3112 to be wound around one side of one or more pulley shafts, and the backward-moving wire BRW to be wound around another side thereof, and when at least one of the first forward-moving wire 3111 and the second forward-moving wire 3112 and the backward-moving wire BRW form a close loop, tension of the entire wire can be easily maintained.

For example, when the end tool is manipulated for a yaw motion or a pitch motion, the wire wound around the shaft corresponding to the motion may be further wound or unwound depending on the direction in which the wire is wound, in this case, when the forward-moving wires 3111 and 3112 and the backward-moving wire BRW are wound in opposite directions, the backward-moving wire BRW may be unwound as much as the forward-moving wires 3111 and 3112 is wound, or the forward-moving wires 3111 and 3112 may be unwound as much as the backward-moving wire BRW is wound, and consequently the overall length of the close loop of the forward-moving wires 3111 and 3112 and the backward-moving wire BRW may be maintained, thereby facilitating the maintenance of tension. At this point, a driving unit (e.g., an actuator) may be used to pull the forward-moving wires 3111 and 3112 or the backward-moving wire BRW to further limit unnecessary movement of the operation member 3140.

As a further example, the close loop of the forward-moving wires 3111 and 3112 and the backward-moving wire BRW may be formed by winding the forward-moving wires 3111 and 3112 and the backward-moving wire BRW around a common pulley fixing the common pulley to a rotation shaft of the rotating driving unit (e.g., an actuator).

Meanwhile, pulling the first forward-moving wire 3111 and the second forward-moving wire 3112 of the present embodiment may cause some rotational force on the end tool 3100 due to the tension thereof, which may cause the jaw 3103 biting the body tissue to lose balance, or cause unnecessary external forces to be applied to the body tissue. In this case, the pulleys and the corresponding pulley shafts of the end tool 3100 may tilt or move slightly, as a result, inner diameter edges of the pulleys may dig into the pulley shafts, thereby increasing friction and tightening the coupling of the pulley and the pulley shaft. Accordingly, friction forces are generated to resist abnormal rotational forces on the end tool 3100, which can improve the reliability and usability of the end tool 3100. In addition, this is applicable even when the backward-moving wire BRW is used, and is applicable to the embodiments described below and the embodiments described above without change.

Meanwhile, when the tension applied to the first forward-moving wire 3111 and the second forward-moving wire 3112 or the backward-moving wire BRW is released, the frictional force may be eliminated together.

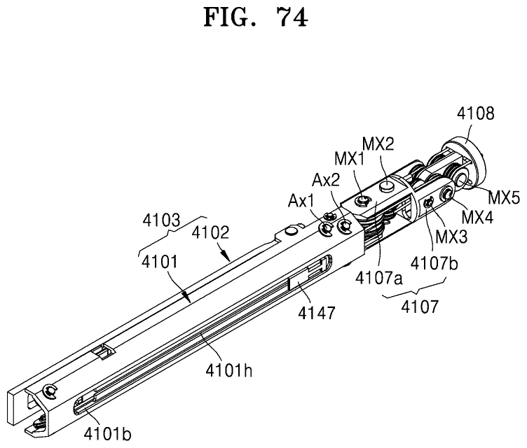

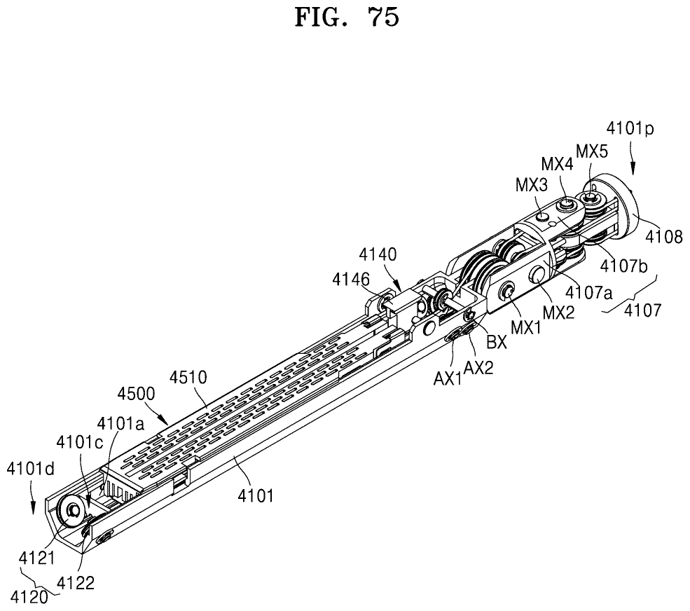

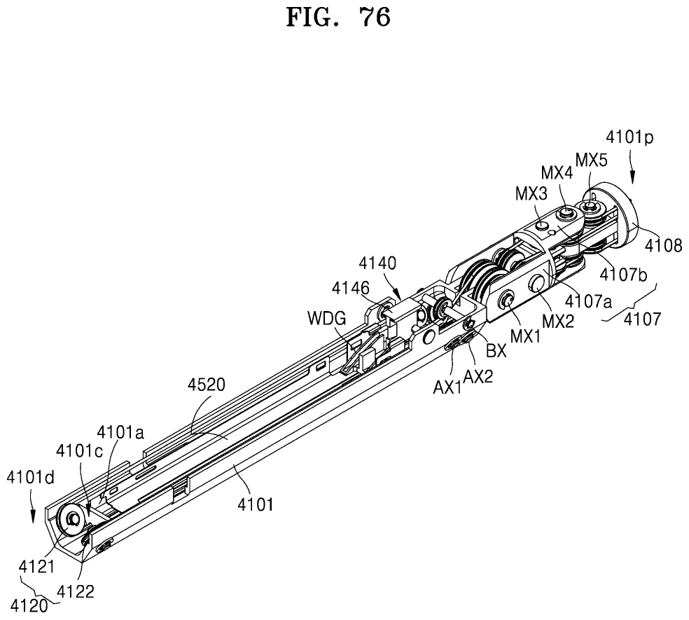

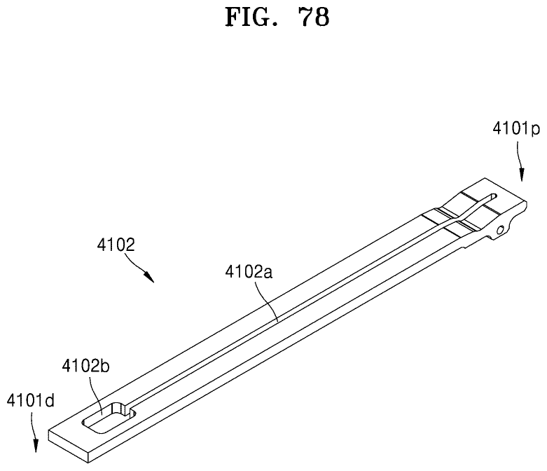

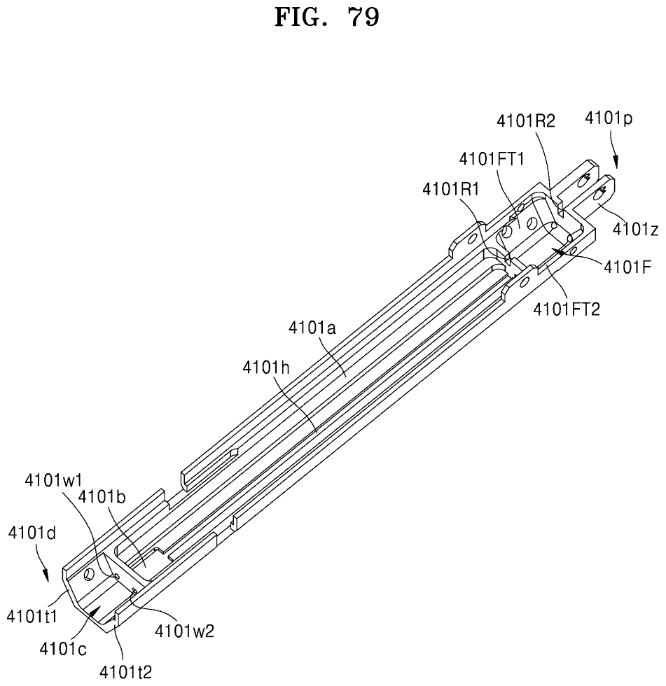

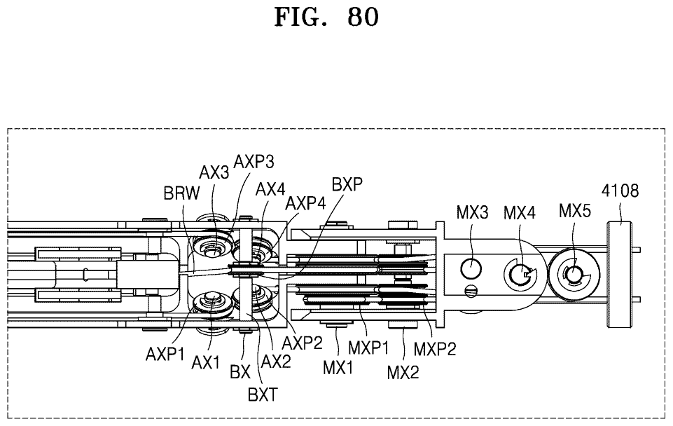

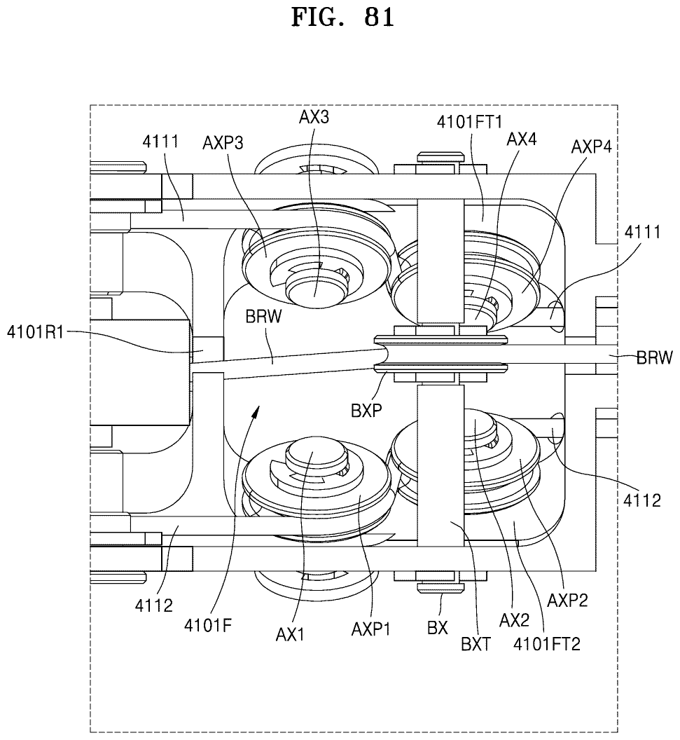

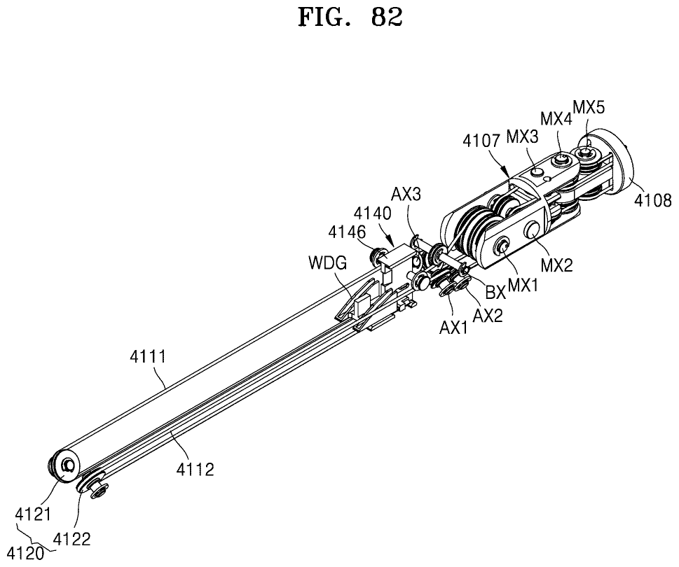

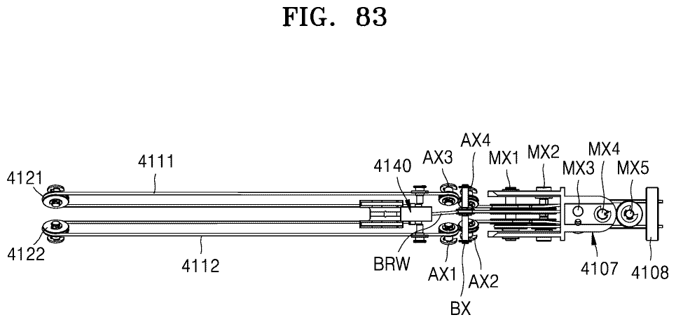

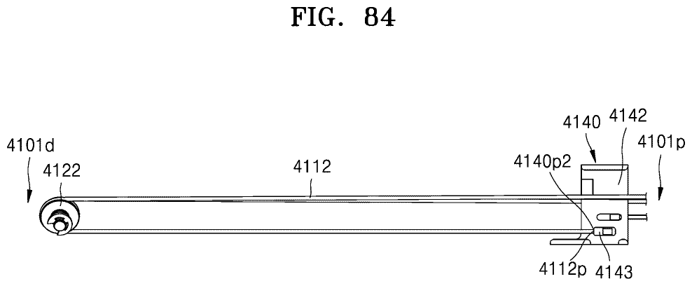

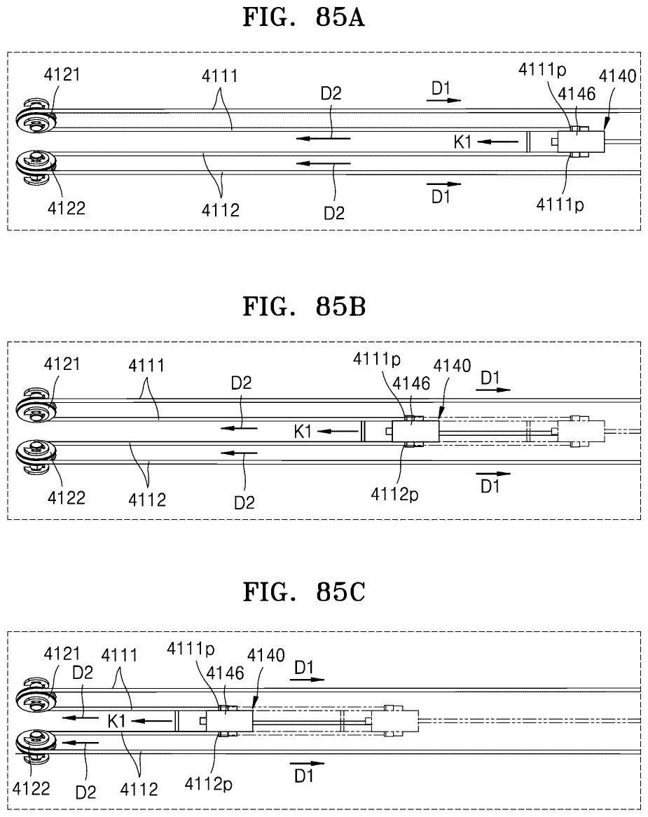

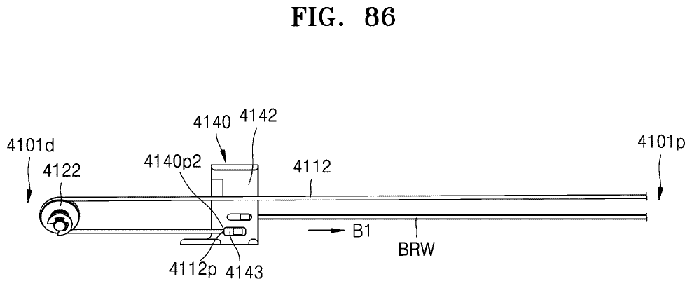

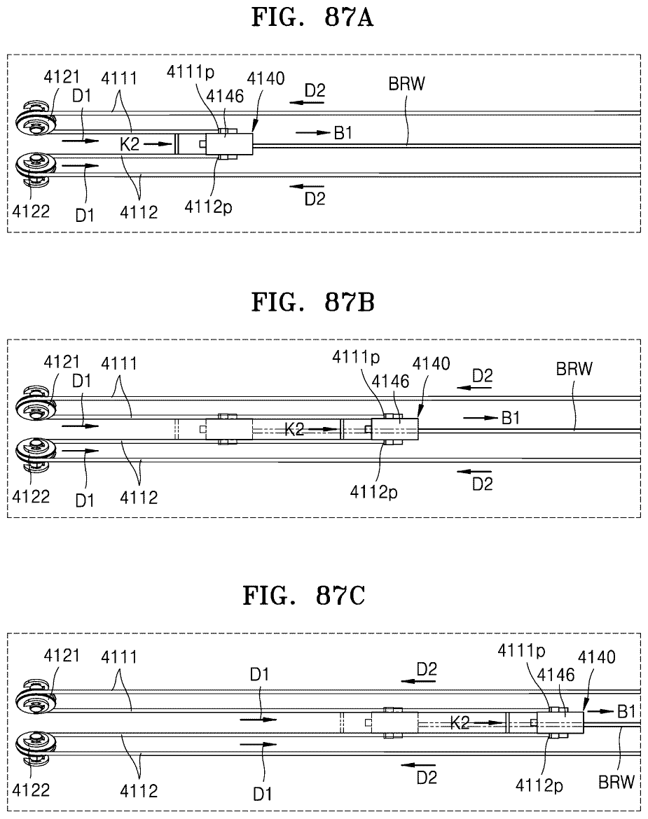

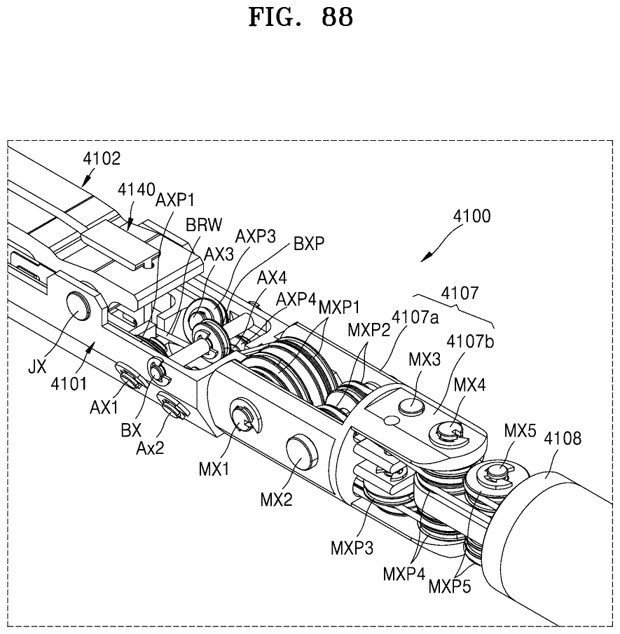

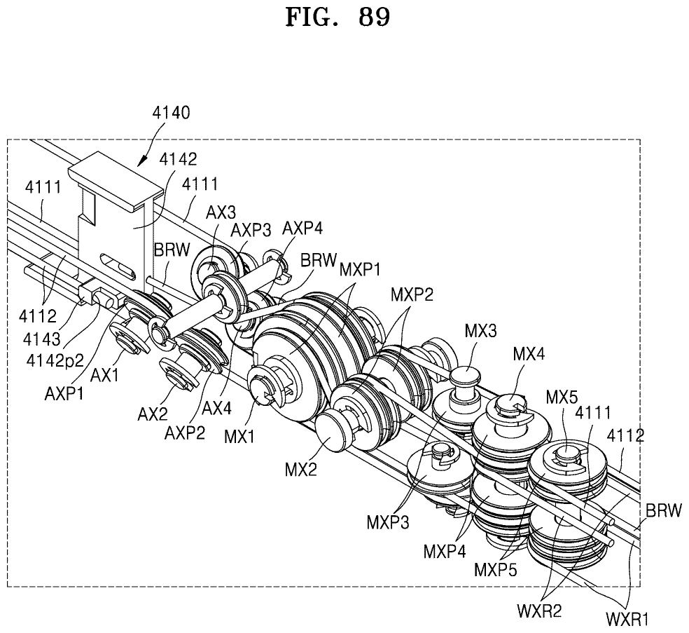

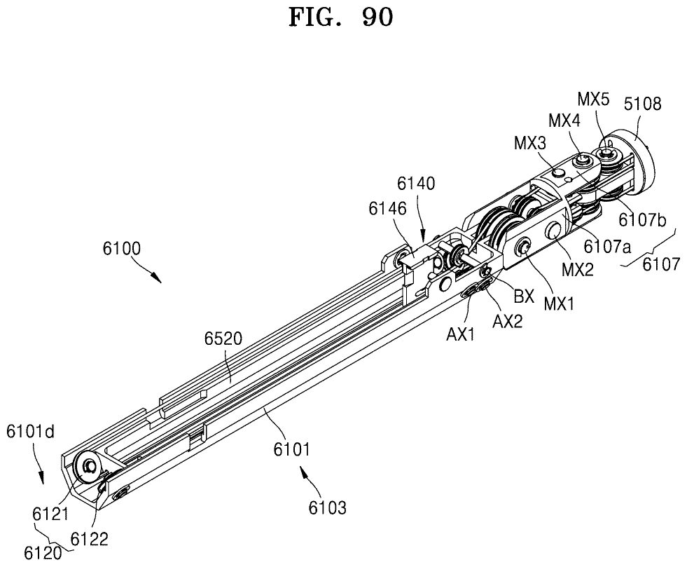

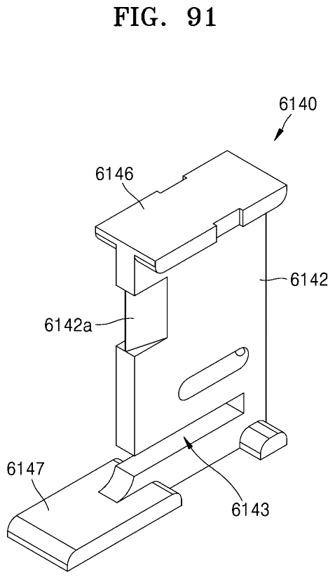

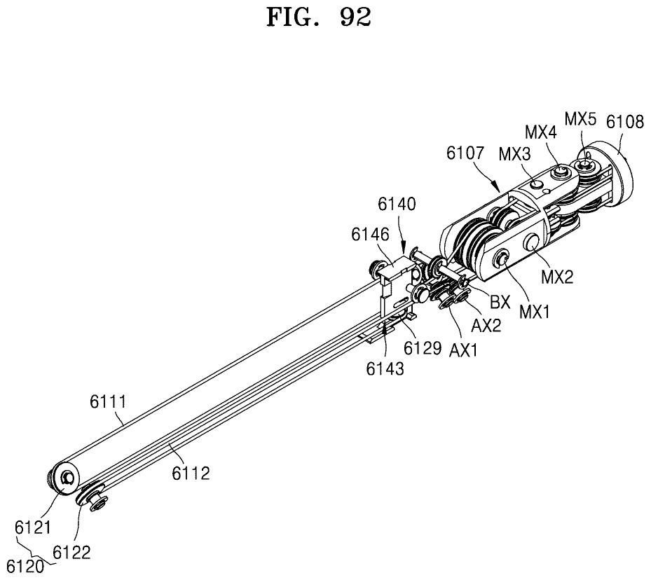

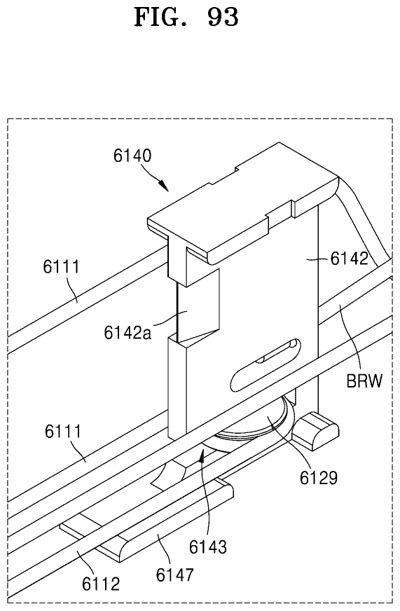

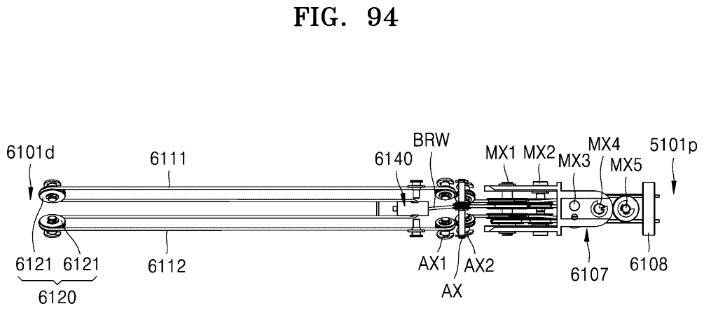

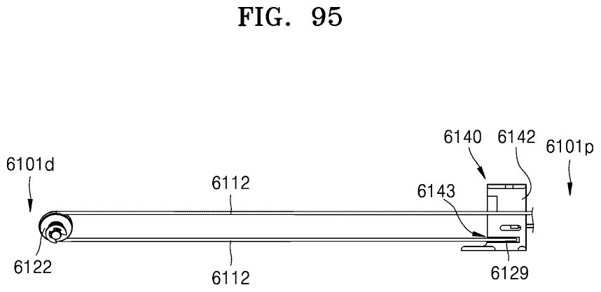

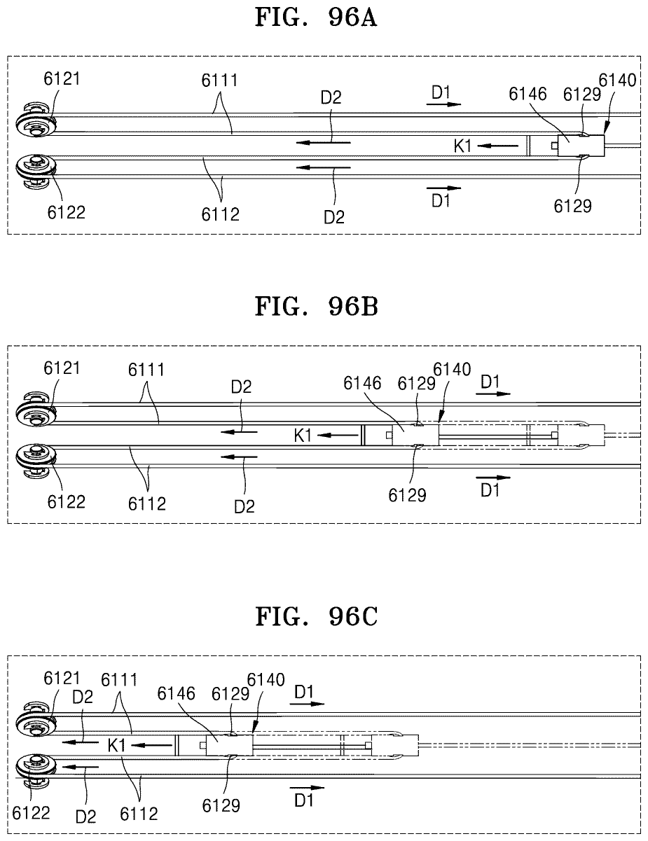

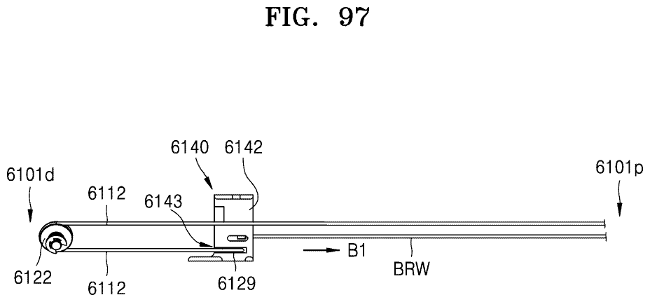

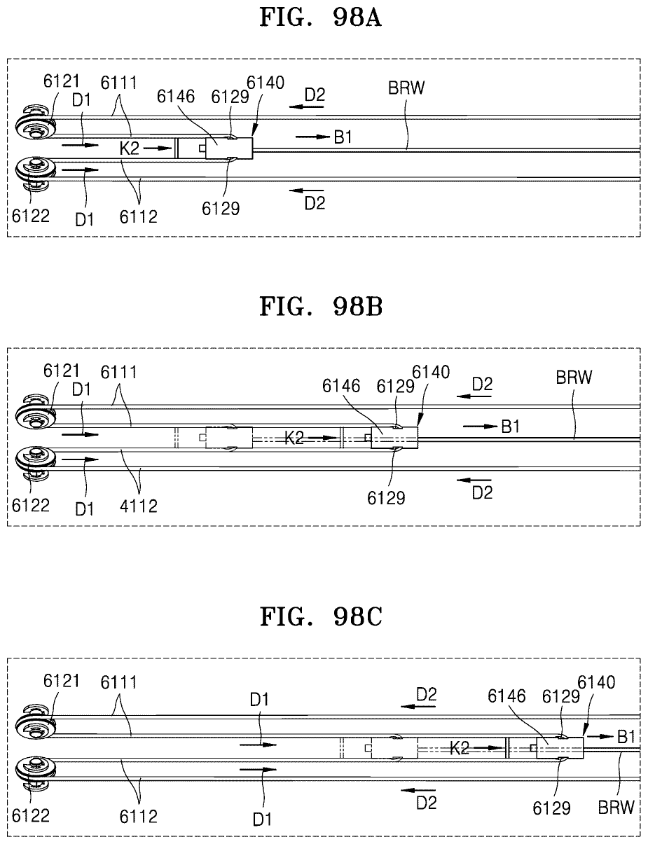

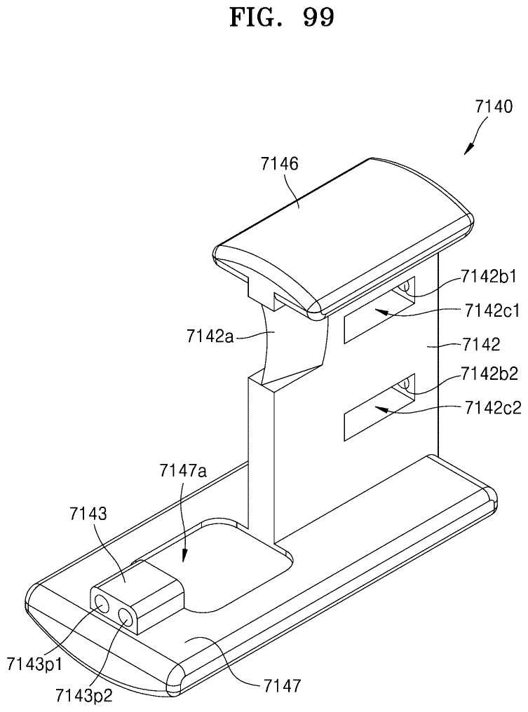

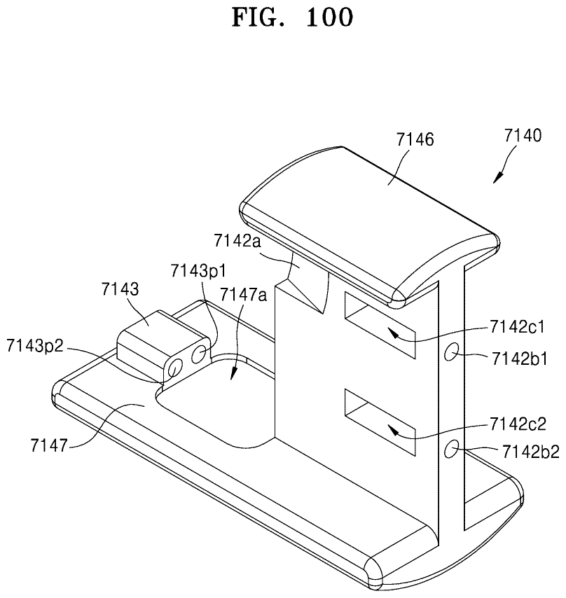

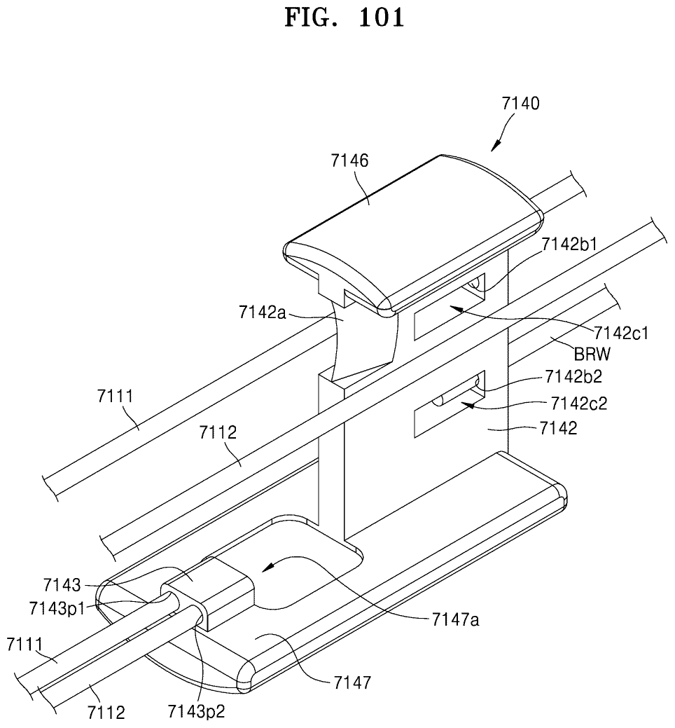

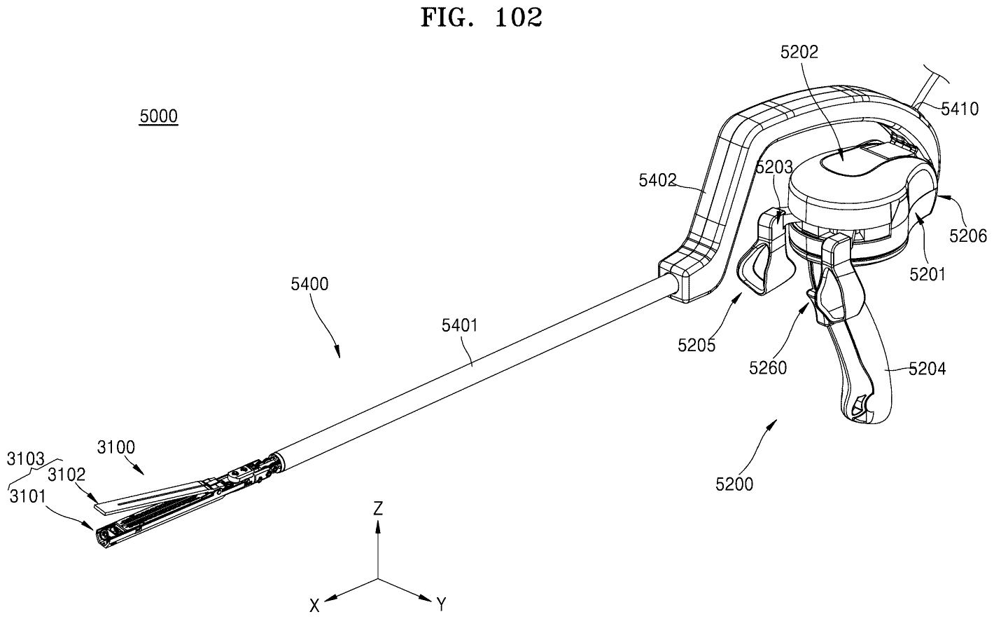

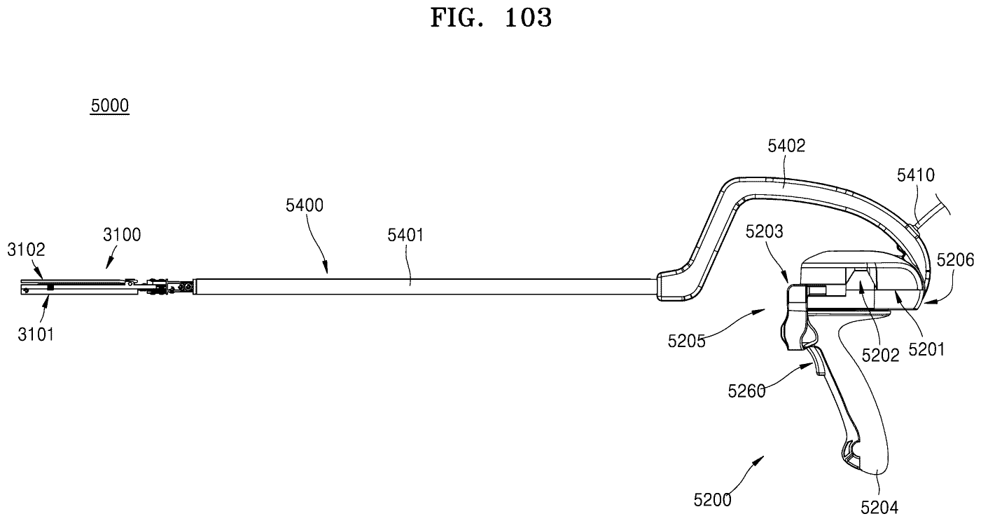

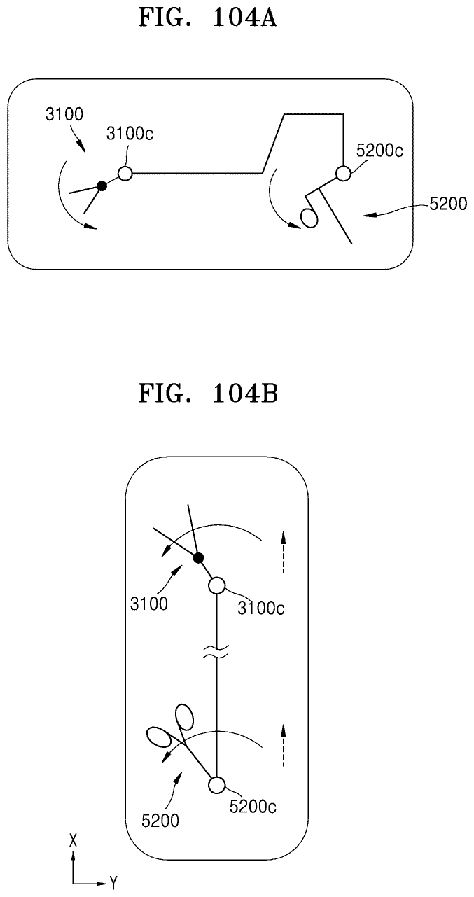

FIG. 73 is a schematic perspective view illustrating an end tool of a surgical instrument according to another embodiment of the present disclosure. FIG. 74 is a perspective view of the end tool of FIG. 73 viewed from another direction. FIG. 75 is a schematic perspective view of the end tool of FIG. 73 with a second jaw removed. FIG. 76 is a schematic perspective view of the end tool of FIG. 75 with a cartridge removed. FIG. 77 is a partial perspective view illustrating one region of FIG. 76. FIG. 78 is a perspective view schematically illustrating the second jaw of the end tool of FIG. 73. FIG. 79 is a perspective view schematically illustrating a first jaw of the end tool of FIG. 73. FIG. 80 is a schematic plan view illustrating a portion of the end tool of FIG. 77. FIG. 81 is a magnified view of a portion of FIG. 80. FIG. 82 is a schematic perspective view for describing an operation member, a fixed pulley, and a forward-moving wire of the end tool of FIG. 73. FIG. 83 is a plan view of FIG. 82. FIGS. 84 and 85A, 85B, and 85C are schematic views for describing an operation of the operation member of the end tool of FIG. 73. FIGS. 86 and 87A, 87B, and 87C are views for describing an optional embodiment in which a backward-moving wire is added to the end tool of FIG. 73. FIGS. 88 and 89 are views for describing a switching pulley, a yaw pulley, and a pitch pulley of the end tool of the surgical instrument of FIG. 73.

An end tool 4100 of a surgical instrument according to the present embodiment may be applied to various surgical instruments and used, for example, may be applied to surgical instruments including the operator 3200 and the connector 3400, such as the surgical instrument 3000 of the above-described embodiment, and to other surgical instruments of various structures, either as it is or with modifications within the same scope as needed.

The end tool 4100 of the present embodiment may include a jaw 4103, a plurality of fixed pulleys 4120, and a plurality of forward-moving wires 4110. The plurality of fixed pulleys 4120 may include two or more pulleys, for example, a first fixed pulley 4121 and a second fixed pulley 4122. The plurality of forward-moving wires 4110 include two or more wires, and may include, for example, a first forward-moving wire 4111 and a second forward-moving wire 4112.

The jaw 4103 may perform various functions, for example, a grip motion, and may include a pair of jaws, e.g., a first jaw 4101 and a second jaw 4102 as a specific example. Here, each of the first jaw 4101 and the second jaw 4102, or a component encompassing the first jaw 4101 and the second jaw 4102 may be referred to as the jaw 4103.

The first jaw 4101 and the second jaw 4102 may be disposed to face each other, may move closer to or move away from each other, and may be formed to rotationally move around, for example, one shaft.

A cartridge 4500 may be disposed to be accommodated in the first jaw 4101, and a plurality of staples are disposed inside the cartridge 4500. When an operation member 4140 receives a force through the forward-moving wire 4110 while the first jaw 4101 and the second jaw 4102 are close to each other, such as when the first jaw 4101 and the second jaw 4102 are closed with the body tissue interposed therebetween, the operation member 4140 may push and raise the staples while moving toward a distal end 4101d of the first jaw 4101, so that stapling may be performed. At this point, one or more clamps 4146 and 4147 of the operation member 4140 may protrude to the outside of the first jaw 4101 and the second jaw 4102, allowing the operation member 4140 to move forward while applying pressure to an outer surface of the first jaw 4101 and the second jaw 4102, which facilitates the smooth progression of a stapling process. In an optional embodiment, the cartridge 4500 may include a case 4520 corresponding to the bottom, and the case 4520 is disposed in the first jaw 4101.

Meanwhile, the operation member 4140 may be used together with a wedge WDG, and the details thereof are the same as those described in the above-described embodiment, and thus a more detailed description thereof will be omitted.

The plurality of fixed pulleys 4120 may be disposed in the first jaw 4101 to be closer to the front of the cartridge 4500, i.e., to the distal end 4101d of the first jaw 4101, than the cartridge 4500.

For example, the first fixed pulley 4121 and the second fixed pulley 4122 may be disposed in a front space 4101c of the first jaw 4101.

In addition, the end tool 4100 of the surgical instrument of the present embodiment may include one or more members, such as joint members, connecting the jaw 4103 to a connector (refer to, for example, 3400 in FIG. 50).

Further, in an optional embodiment, the end tool 4100 may include an end tool hub 4108 and a pitch hub 4107.

The end tool hub 4108 may be disposed at a proximal end 4101p of the end tool 4100 and may be coupled to the connector (refer to, for example, 3400 in FIG. 50).

As an example, the end tool hub 4108 may have a pulley shaft MX4 corresponding thereto, and the pulley shaft MX4 may be a yaw rotation shaft. As a specific example, the end tool 4100 may perform a horizontal rotational motion around the pulley shaft MX4. In addition, one or more pulleys may be disposed to be adjacent to the pulley shaft MX4.

The end tool hub 4108 may be in the form of a bar extending from the center of a surface that corresponds to the connector, for example, a main region with a disk shape, and the pulley shaft MX4 and a pulley shaft MX5 different from the pulley shaft MX4 may further correspond to the bar region.

The pitch hub 4107 is connected to the end tool hub 4108 and the jaw 4103. The pitch hub 4107 may be axially coupled the end tool hub 4108 with respect to one pulley shaft, i.e., the pulley shaft MX4. The pitch hub 4107 may rotationally move around one pulley shaft, i.e., the pulley shaft MX4 while connected to the end tool hub 4108. That is, the end tool 4100 may perform a yaw motion as the pitch hub 4107 rotates around one pulley shaft, i.e., the pulley shaft MX4 with respect to the end tool hub 4108.

Further, the jaw 4103 of the end tool 4100 may be axially coupled to the pitch hub 4107 with respect to one pulley shaft, i.e., a pulley shaft MX1. The jaw 4103 may rotate around one pulley shaft, i.e., the pulley shaft MX1 while connected to the pitch hub 4107. That is, the jaw 4103 of the end tool 4100 may rotate around one pulley shaft, i.e., the pulley shaft MX1 with respect to the pitch hub 4107, thereby performing a pitch motion.

As a result, the pitch motion of the end tool 4100 includes a vertical rotational motion of the jaw 4103 around one pulley shaft, i.e., the pulley shaft MX1 with respect to the pitch hub 4107, and the yaw motion of the end tool 4100 includes a rotational motion of the jaw 4103 coupled to the pitch hub 4107 together with the end tool hub 4108, which occurs as the pitch hub 4107 horizontally rotates around one pulley shaft, i.e., the pulley shaft MX4.

The pitch hub 4107 may include a first hub 4107a and a second hub 4107b.

The first hub 4107a of the pitch hub 4107 may be connected to the jaw 4103. As an example, the first hub 4107a may be elongated to connect to one region of the first jaw 4101, and specifically, may have two bars that are formed side by side to face each other and coupled to each other by placing one region of the first jaw 4101 therebetween.

The second hub 4107b of the pitch hub 4107 may be connected to the end tool hub 4108, for example, may have two bars that are formed side by side to face each other, and may be coupled to each other by placing one region of the end tool hub 4108 therebetween.

As described above, the pulley shaft MX5 is different from one pulley shaft, i.e., the pulley shaft MX4 may be disposed in the end tool hub 4108 to be spaced apart from the pulley shaft MX4 and closer to the connector (refer to, for example, 3400 in FIG. 50) than the pulley shaft MX4. The pulley shaft MX4 and the pulley shaft MX5 may have axes in directions parallel to each other.

A pulley shaft MX2, which is different from the pulley shaft MX1, is disposed on the pitch hub 4107 in a direction adjacent to and parallel to the pulley shaft MX1. In addition, a pulley shaft MX3 and the pulley shaft MX4 may be formed in a direction different from (for example, intersecting or orthogonal to) the direction in which the pulley shaft MX1 and the pulley shaft MX2 are disposed, and may be sequentially disposed in a direction toward (or away from the operation member) the connector (refer to, for example, 3400 in FIG. 50).

The pulley shaft MX4 may be a yaw motion shaft of the end tool 4100, and the pulley shaft MX1 may be a pitch motion shaft.

The pulley shaft MX3 and the pulley shaft MX5 may be yaw auxiliary pulley shafts, and the pulley shaft MX2 may be a pitch auxiliary pulley shaft. One or more driving wires, such as a wire configured to transmit a driving force for a pitch motion or a yaw motion may have at least one region in contact with or wound around the pulley shafts MX1, MX2, MX3, MX4, and MX5.

The pulley shafts MX2, MX3, and MX5 adjacent to the pulley shaft MX4, which is a yaw motion shaft, and the pulley shaft MX1, which is a pitch motion shaft, may control paths along which the driving wires are wound around the pulley shaft MX4 and the pulley shaft MX1 to secure the efficiency of the arrangement of the driving wires and stabilize the paths for transmitting forces through the driving wires.

In addition, at least one region of the forward-moving wire 4110 may be in contact with or wound around the pulley shafts MX1, MX2, MX3, MX4, and MX5.

A more detailed description of the arrangement of the pulley shafts MX1, MX2, MX3, MX4, and MX5 will be provided below.

A plurality of switching pulley shafts AX1, AX2, AX3, and AX4 may be disposed in the end tool 4100, and one or more pulleys corresponding to the switching pulley shafts AX1, AX2, AX3, and AX4 may be disposed.

For example, the switching pulley shafts AX1, AX2, AX3, and AX4 may be disposed in the jaw 4103, specifically, in the proximal end 4101p side of the first jaw 4101, and may be disposed closer to the distal end 4101d of the first jaw 4101 than at least the above-described pulley shafts MX1, MX2, MX3, MX4, and MX5.

The switching pulley shafts AX1, AX2, AX3, and AX4 may have axes that are at least not parallel to each other. For example, first and second switching pulley shafts AX1 and AX2 and third and fourth switching pulley shafts AX3 and AX4 may be disposed in directions intersecting each other, rather than being parallel to each other. The switching pulley shaft AX1 and the switching pulley shaft AX2 may have axes parallel to each other, and the switching pulley shaft AX3 and the switching pulley shaft AX4 may have axes parallel to each other.

The switching pulley shafts AX1, AX2, AX3, and AX4 may be regions where at least one region of the forward-moving wires 4110 is wound or comes into contact to organize and guide the path of the forward-moving wire 4110 before entering the pulley shafts MX1, MX2, MX3, MX4, and MX5. A more detailed description of the arrangement of the switching pulley shafts AX1, AX2, AX3, and AX4 will be provided below.

The first forward-moving wire 4111 and the second forward-moving wire 4112 may be correspondingly wound around the first fixed pulley 4121 and the second fixed pulley 4122 in the first jaw 4101 to be redirected, and connected to the rear of the end tool 4100 via at least one region of each of the switching pulley shafts AX1, AX2, AX3, and AX4 and the pulley shafts MX1, MX2, MX3, MX4, and MX5. Furthermore, the first forward-moving wire 4111 and the second forward-moving wire 4112 may extend to an operator (refer to 3200 of FIG. 50) through the connector (refer to 3400 in FIG. 50) to be precisely controlled. Accordingly, precise motion control of the operation member 4140 may be easily implemented, and details thereof will be described below.

The jaw 4103 of the end tool 4100 will be described in more detail.

FIG. 78 is a perspective view schematically illustrating the second jaw of the end tool of FIG. 73. FIG. 79 is a perspective view schematically illustrating the first jaw of the end tool of FIG. 73.

The second jaw 4102 may be formed in an elongated bar shape as a whole, and for example, the second jaw 4102 may be formed in a rod shape to correspond to the first jaw 4101 in at least one region.

The second jaw 4102 includes a guide groove 4102a. The guide groove 4102a may have a shape elongated in a longitudinal direction of the second jaw 4102.

In an optional embodiment, the second jaw 4102 may include a window 4102b. After operating the operation member 4140 or using the end tool 4100, a first clamp 4146 of the operation member 4140 may be located corresponding to the window 4102b, and the coupled state of the second jaw 4102 and the operation member 4140 may be released. Other more detailed descriptions except for the window 4102b are substantially the same as those of the second jaw of the above-described embodiment, and thus a detailed description thereof will be omitted.

The first jaw 4101 is formed in an elongated bar shape as a whole, and a rotation shaft may be disposed in a proximal end so as to be rotationally movable, and the rotation shaft may correspond to the rotation shaft formed in the second jaw 4102 described above. In addition, the cartridge 4500 (refer to FIG. 75) may be accommodated closer to the distal end 4101d side than the rotation shaft.

For example, the first jaw 4101 may be formed entirely in the form of a hollow box with one surface (upper surface) thereof is removed, such that a cartridge accommodation part 4101a capable of accommodating the cartridge may be formed inside the first jaw 4101. That is, the first jaw 4101 may be formed in a substantially “U” shape in cross section.

A guide groove 4101h may be formed in a bottom surface of the first jaw 4101, the bottom surface opposite to an upper open region formed by removing one surface. Specifically, the guide groove 4101h may be formed to guide a linear motion of the operation member 4140.

The guide groove 4101h may be formed to guide the operation member 4140, and may be a groove formed to pass through a region facing the operation member 4140. Through this, one region of the operation member 4140, such as at least one region of a body 4142 of the operation member 4140, or a second clamp 4147 connected thereto may pass through the guide groove 4101h to exit to the outside of the first jaw 4101. When the operation member 4140 moves forward, the second clamp 4147 may pass through the guide groove 4101h of the first jaw 4101 to be exposed to the outside of the first jaw 4101, and may come into contact with the lower surface of the first jaw 4101 or apply pressure thereto. As the operation member 4140 moves, the second clamp 4147 applies pressure on the lower surface of the first jaw 4101 and the first clamp 4146 applies pressure on the upper surface of the second jaw 4102 such that a gap between the second jaw 4102 and the first jaw 4101 decreases, allowing the second jaw 4102 to naturally remain in a closed state with respect to the first jaw 4101 (refer to FIGS. 20A, 20B, and 20C).

In an optional embodiment, the first jaw 4101 may include a window 4101b. After operating the operation member 4140 or using the end tool 4100, the second clamp 4147 of the operation member 4140 may be located corresponding to the window 4101b, and the coupled state of the first jaw 4101 and the operation member 4140 may be released.

The first jaw 4101 may include the front space 4101c located ahead of the cartridge accommodation part 4101a.

For example, the front space 4101c may be disposed closer to the distal end 4101d of the first jaw 4101 than the cartridge accommodation part 4101a. The plurality of fixed pulleys 4120 may be disposed in the front space 4101c, for example, the first fixed pulley 4121 and the second fixed pulley 4122 may be disposed in the front space 4101c (refer to, for example, FIGS. 73 to 75).

Two outer side surfaces of the front space 4101c include a first side surface 4101t1 and a second side surface 4101t2, and the first fixed pulley 4121 and the second fixed pulley 4122 may be disposed to correspond to the first side surface 4101t1 and the second side surface 4101t2, respectively.

Each of the first side surface 4101t1 and the second side surface 4101t2 may be formed to have an inclined shape. For example, the first side surface 4101t1 and the second side surface 4101t2 may be shaped such that a gap therebetween decreases as it moves downward, instead of being parallel to each other with the same gap. As a specific example, the gap between the first side surface 4101t1 and the second side surface 4101t2 may be formed to be gradually smaller in a direction away from the second jaw 4102. In addition, the first side surface 4101t1 and the second side surface 4101t2 may have shapes symmetrical to each other.

Since the first fixed pulley 4121 and the second fixed pulley 4122 are disposed to correspond to the first side surface 4101t1 and the second side surface 4101t2, respectively, the first fixed pulley 4121 and the second fixed pulley 4122 may be disposed to have an inclined shape such that a gap therebetween decreases in a direction away from the second jaw 4102 (refer to FIG. 73). The first fixed pulley 4121 and the second fixed pulley 4122 may be symmetrically shaped and may have the same size as each other.

In addition, a first path 4101w1 and a second path 4101w2 may be formed adjacent to the front space 4101c. For example, the first path 4101w1 and the second path 4101w2 may have the form of through holes formed in a barrier wall, and may be regions through which the first forward-moving wire 4111 and the second forward-moving wire 4112 pass, respectively.

In an optional embodiment, when a backward-moving wire (refer to, for example, FIG. 68) is disposed, the first jaw 4101 may include one or more rear side paths 4101R1 and 4101R2 corresponding to the backward-moving wire.

In addition, the first jaw 4101 may include a coupling region 4101z in a region adjacent to the proximal end 4101p. The coupling region 4101z is a region coupled to the pitch hub 4107 and may be formed in one or more elongated plates corresponding to, for example, the first hub 4107a of the pitch hub 4107. The coupling region 4101z may be disposed and coupled between two bars formed on the first hub 4107a of the pitch hub 4107.

The first jaw 4101 may include a rear space 4101F reward of the cartridge accommodation part 4101a.

For example, the rear space 4101F may be disposed closer to the proximal end 4101p of the first jaw 4101 than the cartridge accommodation part 4101a. In the rear space 4101F, the switching pulley shafts AX1, AX2, AX3, and AX4 and the plurality of switching pulleys AXP1, AXP2, AXP3, and AXP4 (refer to FIG. 80) corresponding thereto may be disposed.

Two outer side surfaces corresponding to the rear space 4101F include a first side surface 4101FT1 and a second side surface 4101FT2, and the first side surface 4101FT1 and the second side surface 4101FT2 may be formed to face each other and have an inclined shape. For example, the first side surface 4101FT1 and the second side surface 4101FT2 may be shaped such that a gap therebetween decreases as it moves downward, instead of being parallel to each other with the same gap. As a specific example, the gap between the first side surface 4101FT1 and the second side surface 4101FT2 may be formed to be gradually smaller in a direction away from the second jaw 4102. In addition, the first side surface 4101FT1 and the second side surface 4101FT2 may have shapes symmetrical to each other.

The third and fourth switching pulley shafts AX3 and AX4 are disposed in the first side surface 4101FT1, and the first and second switching pulley shafts AX1 and AX2 may be disposed in the second side surface 4101FT2. Further details of the first and second switching pulley shafts AX1 and AX2 and the third and fourth switching pulley shafts AX3 and AX4 disposed in the first side surface 4101FT1 and the second side surface 4101FT2 will be described below.

The operation member 4140 may include the body 4142, the first clamp 4146, and the second clamp 4147. Meanwhile, the operation member 4140 may be used together with the wedge WDG (refer to FIG. 76).

The operation member 4140 is substantially the same as the operation member 3140 of the above-described embodiment, and thus a more detailed description thereof will be omitted.

The rear space 4101F and the first and second switching pulley shafts AX1 and AX2 and the third and fourth switching pulley shafts AX3 and AX4 corresponding thereto will be described in more detail.

FIG. 80 is a schematic plan view illustrating a portion of the end tool of FIG. 77. FIG. 81 is a magnified view of a portion of FIG. 80.

As described above, the first side surface 4101FT1 and the second side surface 4101FT2, which are two outer side surfaces corresponding to the rear space 4101F, may face each other, may each be formed in an inclined shape, and may be shaped such that a gap therebetween decreases as it moves downward, and specifically, may have a symmetrical shape.

The third and fourth switching pulley shafts AX3 and AX4 may be disposed in the first side surface 4101FT1. Accordingly, the third switching pulley shaft AX3 and the fourth switching pulley shaft AX4 may have axes parallel to each other, and the third switching pulley shaft AX3 may be disposed closer to the operation member 4140 than the fourth switching pulley shaft AX4.

The third switching pulley shaft AX3 and the fourth switching pulley shaft AX4 may be disposed at different positions, rather than next to each other based on the heights. For example, the third switching pulley shaft AX3 may be disposed higher than the fourth switching pulley shaft AX4, and as a specific example, the third switching pulley shaft AX3 may be disposed closer to the second jaw 4102 than the fourth switching pulley shaft AX4.

In addition, one or more switching pulleys AXP3 and one or more switching pulleys AXP4 may correspond to the third switching pulley shaft AX3 and the fourth switching pulley shaft AX4, respectively.

A path of the first forward-moving wire 4111 may be guided through the switching pulley AXP3 and the switching pulley AXP4. For example, the first forward-moving wire 4111 may be wound around an upper side of the switching pulley AXP3, and the path of the first forward-moving wire 4111 may progress horizontally or close to horizontally toward a lower side of the switching pulley AXP4, and may sequentially pass through the pulley shaft MX1, which is a pitch rotation shaft, and the pulley shaft MX4, which is a yaw rotation shaft, and may further pass through other pulley shafts MX2, MX3, and MX5 adjacent to the pulley shafts MX1 and MX4 and the plurality of pulleys, including MXP1 and MXP2, corresponding to the pulley shafts MX1, MX2, MX3, MX4, and MX5.

The first and second switching pulley shafts AX1 and AX2 may be disposed in the second side surface 4101FT2. Accordingly, the first switching pulley shaft AX1 and the second switching pulley shaft AX2 may have axes parallel to each other, and the first switching pulley shaft AX1 may be disposed closer to the operation member 4140 than the second switching pulley shaft AX2.

The first switching pulley shaft AX1 and the second switching pulley shaft AX2 may be disposed at different positions, rather than next to each other based on the heights. For example, the first switching pulley shaft AX1 may be disposed higher than the second switching pulley shaft AX2, and as a specific example, the first switching pulley shaft AX1 may be located closer to the second jaw 4102 than the second switching pulley shaft AX2.

In addition, one or more switching pulleys AXP1 and one or more switching pulleys AXP2 may correspond to the first switching pulley shaft AX1 and the second switching pulley shaft AX2, respectively.

A path of the second forward-moving wire 4112 may be guided through the switching pulley AXP1 and the switching pulley AXP2. For example, the second forward-moving wire 4112 may be wound around an upper side of the switching pulley AXP1, and the path of the second forward-moving wire 4112 may progress horizontally or close to horizontally toward a lower side of the switching pulley AXP2, and then, may sequentially pass through the pulley shaft MX1, which is a pitch rotation shaft, and the pulley shaft MX4, which is a yaw rotation shaft, and may further pass through other pulley shafts MX2, MX3, and MX5 adjacent to the pulley shafts MX1 and MX4 and the plurality of pulleys, including MXP1 and MXP2, corresponding to the pulley shafts MX1, MX2, MX3, MX4, and MX5.

In an optional embodiment, when the end tool 4100 of the present embodiment includes a backward-moving wire BRW, a backward-moving wire switching pulley shaft BX may be formed in the rear space 4101F. For example, the backward-moving wire switching pulley shaft BX may be formed to correspond to a support shaft BXT having a shape passing through two outer side surfaces, i.e., the first side surface 4101FT1 and the second side surface 4101FT2.

One or more backward-moving wire switching pulleys BXP corresponding to the backward-moving wire switching pulley shaft BX may be disposed. For example, the backward-moving wire switching pulley BXP may be disposed between the first side surface 4101FT1 and the second side surface 4101FT2 to be spaced apart from the first side surface 4101FT1 and the second side surface 4101FT2.

A path of the backward-moving wire BRW may be guided through the backward-moving wire switching pulley BXP. For example, the backward-moving wire BRW may be wound upwardly around a lower side of the backward-moving wire switching pulley BXP, and then, may sequentially pass through the pulley shaft MX1, which is a pitch rotation shaft, and the pulley shaft MX4, which is a yaw rotation shaft, and may further pass through the other pulley shafts MX2, MX3, and MX5 adjacent to the pulley shafts MX1 and MX4 and the plurality of pulleys, including MXP1 and MXP2, corresponding to the pulley shafts MX1, MX2, MX3, MX4, and MX5.

Accordingly, based on the pulley shaft MX1, which is a pulley shaft related to the joint motion of the end tool 4100, and the pulley MXP1 corresponding thereto, the paths of the first forward-moving wire 4111 and the second forward-moving wire 4112 are guided to the lower side of the pulley MXP1, and the path of the backward-moving wire BRW is guided to the upper side of the pulley MXP1, thereby easily implementing the arrangement of wires that control forward and backward movement of the operation member 4140.

In addition, in an optional embodiment, when the backward-moving wire BRW forms a close loop with the first forward-moving wire 4111 and the second forward-moving wire 4112, tension may be easily maintained across the wire, which is the same as described above.

The arrangement relationship of the operation member 4140, the plurality of forward-moving wires 4110, and the plurality of fixed pulleys 4120 will now be further described.

FIGS. 84 and 85A, 85B, and 85C are schematic views for describing an operation of the operation member of the end tool of FIG. 73.

Referring to FIGS. 84 and 85A, 85B, and 85C, for convenience of description, the first jaw 4101 is excluded, and the first forward-moving wire 4111, the second forward-moving wire 4112, the first fixed pulley 4121, the second fixed pulley 4122, and the operation member 4140 are illustrated.

Based on FIG. 84, the operation member 4140 may move in a leftward direction, i.e., move forward toward the distal end 4101d, and this forward movement is illustrated sequentially in FIGS. 85A, 85B, and 85C.

As shown in FIG. 85A, when the first forward-moving wire 4111 and the second forward-moving wire 4112 are pulled in a first direction D1, one region of each of the first forward-moving wire 4111 and the second forward-moving wire 4112 is pulled in the first direction D1, and thus the regions of the first forward-moving wire 4111 and the second forward-moving wire 4112 emerging from the lower sides of the first fixed pulley 4121 and the second fixed pulley 4122 after being wound around the upper sides thereof move in a second direction D2, which is the opposite direction of the first direction D1. Accordingly, forces of the first forward-moving wire 4111 and the second forward-moving wire 4112 are transmitted respectively to a first connection region and a second connection region of the operation member 4140, which are respectively connected to the first forward-moving wire 4111 and the second forward-moving wire 4112, and the forces cause the operation member 4140 to move in a direction K1, which is the same direction as the second direction D2, i.e., move forward, thereby positioning the operation member 4140 in an advanced position shown in FIG. 85B.

Thereafter, as shown in FIG. 85B, when the first forward-moving wire 4111 and the second forward-moving wire 4112 are pulled further in the first direction D1, one region of the first forward-moving wire 4111 and the second forward-moving wire 4112 is pulled further in the first direction D1, and thus the regions of the first forward-moving wire 4111 and the second forward-moving wire 4112 emerging from the lower sides of the first fixed pulley 4121 and the second fixed pulley 4122 after being wound around the upper sides thereof move further in the second direction D2, which is the opposite direction of the first direction D1. Accordingly, forces of the first forward-moving wire 4111 and the second forward-moving wire 4112 are transmitted respectively to the first connection region and the second connection region of the operation member 4140 connected to the first forward-moving wire 4111 and the second forward-moving wire 4112, and the forces cause the operation member 4140 to move further in the direction K1, which is the same direction as the second direction D2, i.e., move forward to a further advanced position than in FIG. 85B, thereby positioning the operation member 4140 in the further advanced position shown in FIG. 85C.

Although not shown in the drawings, it will be appreciated of course that the form corresponding to the side view illustrating the operation of the operation member of FIGS. 20A, 20B, and 20C may be applied to the end tool 4100 of the present embodiment either as is or with appropriate modifications.

FIGS. 86 and 87A, 87B, and 87C are views for describing an optional embodiment in which the backward-moving wire is added to the end tool of FIG. 73.

Referring to FIGS. 86 and 87A, 87B, and 87C, the end tool of the present embodiment may further include the backward-moving wire BRW.

For example, the structure of FIG. 86 may be the structure of FIG. 84 with the addition of the backward-moving wire BRW.

Referring to FIGS. 86 and 87A, 87B, and 87C, for convenience of description, the first jaw 4101 is excluded, and the first forward-moving wire 4111, the second forward-moving wire 4112, the first fixed pulley 4121, the second fixed pulley 4122, the operation member 4140, and the backward-moving wire BRW are illustrated.

Based on FIG. 86, the operation member 4140 may move in a rightward direction, i.e., move backward toward the proximal end 4101p, and this backward movement is illustrated sequentially in FIGS. 87A, 87B, and 87C.

The backward-moving wire BRW may be connected to one region of the operation member 4140 and may be connected, for example, to a rear side of the operation member, specifically, to a surface of the body 4142, which is opposite the region in which an edge of a blade region 4142a is formed.

A driving part or driving transmission part (e.g., a wire, a pulley, or the like) capable of pulling the backward-moving wire BRW may be connected to the backward-moving wire BRW, and the backward-moving wire BRW may be operated according to manual or automatic manipulation. For example, the backward-moving wire BRW may be pulled by the operator (refer to, for example, 3200 of FIG. 50).

By pulling the backward-moving wire BRW. The operation member 4140 may move backward.

For example, as shown in FIG. 87A, the backward-moving wire BRW is pulled in a reverse direction B1 in a state in which the operation member 4140 is located adjacent to the distal end 4101d of the first jaw 4101, the operation member 4140 connected to the backward-moving wire BRW moves backward in a direction K2, which is the same direction as the reverse direction B1.

At this point, the first forward-moving wire 4111 and the second forward-moving wire 4112 may be in a state in which a pulling force is not applied.

When the operation member 4140 moves backward (in the direction K2), the region of the first forward-moving wire 4111 and the second forward-moving wire 4112 connected to the first connection region and the second connection region of the operation member 4140 may move in the first direction D1, which is the same direction as the direction K2, and the regions of the first forward-moving wire 4111 and the second forward-moving wire 4112, which are wound around the lower sides of the first fixed pulley 4121 and the second fixed pulley 4122 and disposed on the upper sides thereof, may move in the second direction D2, which is the opposite direction of the direction K2. Accordingly, the operation member 4140 is in a position as shown in FIG. 87B, having moved backward to be closer to the proximal end 4101p compared to its position in FIG. 87A.

Thereafter, as shown in FIG. 87B, when the backward-moving wire BRW is pulled further in the reverse direction B1, the operation member 4140 connected to the backward-moving wire BRW moves backward in the direction K2, which is the same direction as the reverse direction B1. At this point, the first forward-moving wire 4111 and the second forward-moving wire 4112 may be in a state in which a pulling force is not applied. The regions of the first forward-moving wire 4111 and the second forward-moving wire 4112 connected to the first connection region and the second connection region of the operation member 4140 move in the first direction D1, which is the same direction as the direction K2, and the regions of the first forward-moving wire 4111 and the second forward-moving wire 4112, which are wound around the lower sides of the first fixed pulley 4121 and the second fixed pulley 4122 and disposed on the upper sides thereof, may move in the second direction D2, which is the opposite direction of the direction K2. Accordingly, the operation member 4140 is in a position as shown in FIG. 87C, having moved backward to be closer to the proximal end 4101p compared to its position in FIG. 87B.

Further, although not shown in the drawings, it is of course possible that the configuration of FIG. 27 described above may be optionally applied to the end tool 4100 of the present embodiment.

The cartridge 4500 accommodated in the end tool 4100 and a staple motion are substantially the same as those described in the end tool 3100 of the above-described embodiment, and thus detailed descriptions thereof will be omitted.

FIGS. 88 and 89 are views for describing the switching pulley, the yaw pulley, and the pitch pulley of the end tool of the surgical instrument of FIG. 73.

As described above, the end tool 4100 may be connected to the connector (refer to, for example, 3400 in FIG. 50), and the end tool 4100 may rotationally move around one shaft and also rotationally move around another shaft with respect to the connector.

For example, the end tool 4100 may perform a pitch motion, i.e., a vertical rotational motion, and the end tool 4100 may perform a yaw motion, i.e., a horizontal rotational motion. A rotation shaft of the pitch motion and a rotation shaft of the yaw motion may be located in directions that may intersect or be perpendicular to each other.

As an example, the end tool 4100 may include one or more members, such as joint members, that connect the jaw 4103 to the connector, and may include the end tool hub 4108 and the pitch hub 4107.

The end tool hub 4108 may be disposed to connect the end tool 4100 to a straight part (refer to, for example, 3401 in FIG. 50) of the connector (refer to, for example, 3400 in FIG. 50). As an example, the end tool hub 4108 may have the pulley shaft MX4 corresponding thereto, and the pulley shaft MX4 may be a rotation shaft of the yaw motion. As a specific example, the end tool 4100 may rotationally move around the pulley shaft MX4, the pitch hub 4107 may rotationally move around the pulley shaft MX4, and the jaw 4103 may be connected to the pitch hub 4107 to perform a rotational motion, i.e., a yaw motion, around the pulley shaft MX4 integrally with the pitch hub 4107.

The pitch hub 4107 is connected to the end tool hub 4108 and the jaw 4103, and may rotationally move around the pulley shaft MX4 by being axially coupled to the end tool hub 4108 by the pulley shaft MX4. Further, the jaw 4103 may be axially coupled to the pitch hub 4107 with respect to one pulley shaft, i.e., the pulley shaft MX1. The jaw 4103 may perform a rotational motion, i.e., a pitch motion, around one pulley shaft, i.e., the pulley shaft MX1 while connected to the pitch hub 4107.

An auxiliary pulley shaft may be additionally disposed together with these rotation shafts, i.e., the pulley shafts for joint motion of the end tool 4100, such as the pulley shaft MX4 for yaw motion and the pulley shaft MX1 for pitch motion.

For example, the pulley shaft MX2 different from the pulley shaft MX1 is disposed in the pitch hub 4107 to be adjacent to and parallel to the pulley shaft MX1. The pulley shaft MX2 may have an axis oriented parallel to the pulley shaft MX1, and may be disposed further away from the operation member 4140 than the pulley shaft MX1, i.e., closer to the pulley shaft MX4

In addition, the pulley shaft MX3 may be disposed adjacent to the pulley shaft MX4. In addition, the pulley shaft MX5 may be further disposed.

For example, the pulley shaft MX3 and the pulley shaft MX5 may be disposed on both sides of the pulley shaft MX4 interposed therebetween, and the pulley shaft MX3 and the pulley shaft MX5 may have axes oriented parallel to the pulley shaft MX4.

As a specific example, the pulley shaft MX3 may be disposed between the pulley shaft MX2 and the pulley shaft MX4, and may have an axis intersecting or orthogonal to the pulley shaft MX2 and the pulley shaft MX1. The pulley shaft MX5 may be disposed further away from the operation member 4140 than the pulley shaft MX4.

As described above, the plurality of switching pulley shafts AX1, AX2, AX3, and AX4 may be disposed in the end tool 4100, for example, may be disposed in the rear space 4101F of the first jaw 4101 to be closer to the operation member 4140 than the pulley shafts MX1, MX2, MX3, MX4, and MX5.

One or more pulleys may be disposed on the plurality of switching pulley shafts AX1, AX2, AX3, and AX4 and the pulley shafts MX1, MX2, MX3, MX4, and MX5.

The third switching pulley shaft AX3 and the fourth switching pulley shaft AX4 may be disposed in the first side surface 4101FT1 of the first jaw 4101, and one or more switching pulleys AXP3 and one or more switching pulleys AXP4 may correspond to the third switching pulley shaft AX3 and the fourth switching pulley shaft AX4, respectively.

The first switching pulley shaft AX1 and the second switching pulley shaft AX2 may be disposed in the second side surface 4101FT2 of the first jaw 4101, and one or more switching pulleys AXP1 and one or more switching pulleys AXP2 may correspond to the first switching pulley shaft AX1 and the second switching pulley shaft AX2, respectively.

The first forward-moving wire 4111 may enter the upper side of the switching pulley AXP3 to be wound around the switching pulleys AXP3, and enter the lower side of the switching pulley AXP4. The path of the first forward-moving wire 4111 may progress horizontally or close to horizontally and may sequentially pass through the pulley shaft MX1, which is a pitch rotation shaft.

The second forward-moving wire 4112 may enter the upper side of the switching pulley AXP1 to be wound around the switching pulleys AXP1, and enter the lower side of the switching pulley AXP2. The path of the second forward-moving wire 4112 may progress horizontally or close to horizontally and may sequentially pass through the pulley shaft MX1, which is a pitch rotation shaft.

Accordingly, the first forward-moving wire 4111 and the second forward-moving wire 4112 are spaced apart from each other and have positions parallel to each other, and paths of the first forward-moving wire 4111 and the second forward-moving wire 4112 transition from the upper side to the lower side, becoming horizontal in at least one region corresponding to the lower side of the pulley shaft MX1. In addition, such a path structure also allows the first forward-moving wire 4111 and the second forward-moving wire 4112 to parallelly pass through the sequentially placed pulley shafts and pulleys, facilitating precise simultaneous control of the first forward-moving wire 4111 and the second forward-moving wire 4112.

The switching pulleys AXP1 and AXP2 may be shaped to be symmetrical with the switching pulleys AXP3 and AXP4 to facilitate path guidance of the first forward-moving wire 4111 and the second forward-moving wire 4112.

One or more pulleys MXP1 are disposed to correspond to the pulley shaft MX1, and one or more pulleys MXP2 correspond to the pulley shaft MX2 are disposed to adjacent the one or more pulleys MXP1. The pulley MXP1 and the pulley MXP2 may have axes parallel to each other.

For example, the pulley MXP1 and the pulley MXP2 are disposed in the first hub 4107a of the pitch hub 4107. One or more pulleys MXP2 guide the paths along which the wires, which are disposed to correspond to one or more pulleys MXP1, are driven, ensuring that the wires have a clear path to the pulley shaft MX3 and a pulley MXP3 corresponding thereto.

Further, one or more pulleys MXP3 are disposed to correspond to the pulley shaft MX3, and one or more pulleys MXP4 corresponding to the pulley shaft MX4 are disposed adjacent to the one or more pulleys MXP4. For example, the pulley MXP3 and the pulley MXP4 are disposed in the second hub 4107b of the pitch hub 4107. In addition, one or more pulleys MXP5 may be disposed to correspond to the pulley shaft MX5.

The pulley MXP3, the pulley MXP4, and the pulley MXP5 may have axes that are parallel to each other and intersect or orthogonal the axes of the pulley MXP1 and the pulley MXP2.

Meanwhile, by precisely controlling the paths of the first forward-moving wire 4111 and the second forward-moving wire 4112 as shown in FIGS. 88 and 89, the drive efficiency and control characteristics of the operation member 4140 can be maximized through the first forward-moving wire 4111 and the second forward-moving wire 4112.

As described above, the plurality of switching pulleys AXP1, AXP2, AXP3, and AXP4 are disposed further forward than the pulley shaft MX1 and the pulley shaft MX4, which are respectively for two joint motions such as a pitch motion and a yaw motion of the end tool 4100, i.e., disposed closer to the operation member 4140 than the above pulleys.

As a specific example, the plurality of switching pulleys AXP1, AXP2, AXP3, and AXP4 are disposed further forward than the pulley MXP1, which is a pulley corresponding to a pitch motion, the pulley MXP4, which is a pulley corresponding to a yaw motion, pulley MXP3 and the pulley MXP5, which are pulleys corresponding to the yaw auxiliary pulley, and the pulley MXP2 that is a pitch auxiliary pulley, i.e., disposed closer to the operation member 4140 than the above pulleys.

As a result, the plurality of switching pulleys AXP1, AXP2, AXP3, and AXP4 are disposed further forward than the pulley MXP1, the pulley MXP2, the pulley MXP3, the pulley MXP4, and the pulley MXP5, i.e., disposed closer to the operation member 4140 than the above pulleys.

Accordingly, the paths of the first forward-moving wire 4111 and the second forward-moving wire 4112 transition from the upper side to the lower side at positions parallel to each other, becoming horizontal in one region of the lower side of the pulley MXP1. In addition, the first forward-moving wire 4111 and the second forward-moving wire 4112 may emerge from being wound around the upper side of the pulley MXP2 after passing through the lower side of the pulley MXP1 and the lower side of the pulley MXP2, and may be directed toward the connector (refer to, for example, 3400 in FIG. 50) while crossing each other through different regions of the upper sides of the pulley MXP3, the pulley MXP4, and the pulley MXP5.

That is, by first allowing the first forward-moving wire 4111 and the second forward-moving wire 4112 to be placed side by side corresponding to one side (e.g., the lower side) of the pulley MXP1 through the plurality of switching pulleys AXP1, AXP2, AXP3, and AXP4, the paths of the first forward-moving wire 4111 and the second forward-moving wire 4112 can be easily guided by simultaneously corresponding to the rotation shafts and the pulleys and their auxiliary pulleys for the joint motion of the end tool 4100, so that the accuracy and stability of the forward movement of the operation member 4140 can be improved.

In addition, through the structure in which the paths of the first forward-moving wire 4111 and the second forward-moving wire 4112, which are collected together, transition from the upper side to the lower side, the pulley shaft MX1, which is a pitch rotational motion shaft, and the pulley shaft MX4, which is a yaw motion shaft can be sequentially disposed around the operation member 4140, and this arrangement facilitates the easy implementation and modification of the structure, allowing for sequential disposition of the end tool, the pitch joint, and the yaw joint.

In an optional embodiment, as described above, the backward-moving wire BRW may be further disposed, in which case the backward-moving wire BRW corresponds to the backward-moving wire switching pulley BXP. The backward-moving wire switching pulley BXP may be disposed between and spaced apart from the plurality of switching pulleys AXP1, AXP2, AXP3, and AXP4. For example, the backward-moving wire switching pulley BXP may be disposed to correspond to a space between the plurality of switching pulleys AXP1 and AXP2 and the plurality of switching pulleys AXP3 and AXP4.

The backward-moving wire BRW corresponds to the backward-moving wire switching pulley BXP and is guided so as not to overlap the paths of the first forward-moving wire 4111 and the second forward-moving wire 4112. For example, the backward-moving wire BRW may emerge from being wound around the upper side of the pulley MXP2 after passing through the upper side of the pulley MXP1 and the lower side of the pulley MXP2, and may be directed toward the connector (refer to, for example, 3400 in FIG. 50) while passing through the lower sides of the pulley MXP3, the pulley MXP4, and the pulley MXP5. This allows the paths of the backward-moving wire BRW and the first forward-moving wire and the second forward-moving wire 4111 and 4112 to be placed in different planes, so that the efficiency and stability of the arrangement of the backward-moving wire BRW and the first forward-moving wire and the second forward-moving wire 4111 and 4112 can be improved, and precise control over the application of forces to the backward-moving wire BRW and the first forward-moving wire and the second forward-moving wire 4111 and 4112 can be achieved.

In addition, in an optional embodiment, when the backward-moving wire BRW and at least one of the first forward-moving wire 4111 and the second forward-moving wire 4112 form a close loop, maintaining tension throughout the entire wire becomes easier.

In addition, by arranging the first forward-moving wire 4111, the second forward-moving wire 4112, and the backward-moving wire BRW in a stable and balanced manner, uneven tension on the first forward-moving wire and the second forward-moving wire 4111 and 4112 and the backward-moving wire BRW may be reduced or prevented during the arrangement of driving wires WXR1 and WXR2 for a pitch or yaw motion and force application to the driving wires WXR1 and WXR2 for movement.

FIG. 90 is a schematic perspective view illustrating an end tool of a surgical instrument according to another embodiment of the present disclosure. FIG. 91 is a perspective view schematically illustrating an operation member disposed in the end tool of FIG. 90. FIG. 92 is a schematic perspective view for describing the operation member, a fixed pulley, and a forward-moving wire of the end tool of FIG. 90. FIG. 93 is a magnified view illustrating one region of FIG. 92. FIG. 94 is a plan view of FIG. 92. FIGS. 95 and 96A, 96B, and 96C are schematic views for describing an operation of the operation member of the end tool of FIG. 90. FIGS. 97 and 98A, 98B, and 98C are views for describing an optional embodiment in which a backward-moving wire is added to the end tool of FIG. 90.

An end tool 6100 of a surgical instrument according to the present embodiment may be applied to various surgical instruments and used, for example, may be applied to surgical instruments including the operator 3200 and the connector 3400, such as the surgical instrument 3000 of the above-described embodiment, and to other surgical instruments of various structures, either as it is or with modifications within the same scope as needed.

In addition, in the end tool 6100 of the present embodiment, the interrelationships between an operation member 6140 and a first forward-moving wire 6111 and a second forward-moving wire 6112 differ somewhat from those in the previously described embodiments, and thus these differences will be mainly described.

The end tool 6100 of the present embodiment may include a jaw 6103, a plurality of fixed pulleys 6120, and a plurality of forward-moving wires 6110. The plurality of fixed pulleys 6120 may include two or more pulleys, for example, a first fixed pulley 6121 and a second fixed pulley 6122. The plurality of forward-moving wires 6110 include two or more wires, and may include, for example, the first forward-moving wire 6111 and the second forward-moving wire 6112.

The jaw 6103 may perform various functions, for example, a grip motion, and may include a pair of jaws, e.g., a first jaw 6101 and a second jaw (not shown) as a specific example. Here, each of the first jaw 6101 and the second jaw (not shown), or a component encompassing the first jaw 6101 and the second jaw (not shown) may be referred to as the jaw 6103.

A cartridge (not shown) may be disposed to be accommodated in the first jaw 6101. For example, the cartridge may include a case 6520 corresponding to the bottom, and the case 6520 may be disposed in the first jaw 6101. The cartridge may be substantially the same as the cartridge in the previously described embodiments, or may be a variation of the cartridge described in the previously described embodiments within a similar scope, and thus will not be described in detail.

The plurality of fixed pulleys 6120 may be disposed in the first jaw 6101. For example, the first fixed pulley 6121 and the second fixed pulley 6122 may be disposed in a front space 6101c of the first jaw 6101. The contents of the first fixed pulley 6121 and the second fixed pulley 6122 are substantially the same as those described in the above-described embodiment, and thus detailed descriptions thereof will be omitted.

Further, in an optional embodiment, the end tool 6100 may include an end tool hub 6108 and a pitch hub 6107.

As an example, the end tool hub 6108 may have a pulley shaft MX4 corresponding thereto, and the pulley shaft MX4 may be a yaw rotation shaft. As a specific example, the end tool 6100 may perform a horizontal rotational motion around the pulley shaft MX4. In addition, one or more pulleys may be disposed to be adjacent to the pulley shaft MX4.

Further, the jaw 6103 of the end tool 6100 may be axially coupled to the pitch hub 6107 with respect to one pulley shaft, i.e., a pulley shaft MX1. A pulley shaft MX2, which is different from the pulley shaft MX1, is disposed in the pitch hub 6107 in a direction adjacent to and parallel to the pulley shaft MX1. In addition, a pulley shaft MX3 and the pulley shaft MX4 may be formed in a direction different from (for example, intersecting or orthogonal to) the direction in which the pulley shaft MX1 and the pulley shaft MX2 are disposed, and may be sequentially disposed in a direction toward (or away from the operation member) the connector (refer to, for example, 3400 in FIG. 50).

The contents related to the pitch hub 6107, the end tool hub 6108, the pitch hub 6107, the pulley shaft MX1, the pulley shaft MX2, the pulley shaft MX3, the pulley shaft MX4, and the pulley shaft MX5 are substantially the same as those in the previously described embodiments, and thus detailed descriptions thereof will be omitted.

A plurality of switching pulley shafts AX1, AX2, AX3, and AX4 may be disposed in the end tool 6100, and one or more pulleys corresponding to the switching pulley shafts AX1, AX2, AX3, and AX4 may be disposed in the end tool 6100. The contents of the pulleys corresponding to the switching pulley shaft are substantially the same as those in the previously described embodiments, and thus detailed descriptions thereof will be omitted.

The first forward-moving wire 6111 and the second forward-moving wire 6112 may be correspondingly wound around the first fixed pulley 6121 and the second fixed pulley 6122 in the first jaw 6101 to be redirected, and connected to the rear of the end tool 6100 via at least one region of each of the switching pulley shafts AX1, AX2, AX3, and AX4 and the pulley shafts MX1, MX2, MX3, MX4, and MX5. Furthermore, the first forward-moving wire 6111 and the second forward-moving wire 6112 may further extend to an operator (refer to 3200 of FIG. 50) via the connector (refer to 3400 in FIG. 50) to be precisely controlled. Accordingly, precise motion control of the operation member 6140 can be easily implemented.

For example, the first jaw 6101 may be formed entirely in the form of a hollow box with one surface (upper surface) thereof is removed, such that a cartridge accommodation part 6101a capable of accommodating the cartridge may be formed inside the first jaw 6101. In addition, the first jaw 6101 may include the front space 6101c located ahead of the cartridge accommodation part 6101a.

Other contents of the jaw 6103 including the first jaw 6101 are substantially the same as those in the previously described embodiments, or may be partially modified to the extent necessary, and thus detailed descriptions thereof will be omitted.”

An operation member 6140 will now be described in detail.

FIG. 91 is a perspective view schematically illustrating the operation member disposed in the end tool of FIG. 90. FIG. 92 is a schematic perspective view for describing the operation member, the fixed pulley, and the forward-moving wire of the end tool of FIG. 90. FIG. 93 is a magnified view illustrating one region of FIG. 92. FIG. 94 is a plan view of FIG. 92.

The operation member 6140 may include a body 6142, a first clamp 6146, and a second clamp 6147. Meanwhile, the operation member 6140 may be used together with a wedge (refer to WDG of FIGS. 54 and 55).

The body 6142 may be in the form of an elongated column, such as a plate-shaped column. In addition, a blade region 6142a may be formed in one region of the body 6142, and an edge sharply formed to cut tissue may be formed in the blade region 6142a. The tissue disposed between the first jaw 6101 and the second jaw (not shown) may be cut as at least a portion of the edge formed in one region of the body 6142 is withdrawn to the outside of the first jaw 6101 and the cartridge (not shown).

The first clamp 6146 may be formed in one region of the body 6142, and the second clamp 6147 may be formed in another region different from the one region. For example, the body 6142 may be disposed between the first clamp 6146 and the second clamp 6147, and the contents of which are substantially the same as those in the previously described embodiments.

A connection pulley disposition region 6143 may be formed in one region of the body 6142. The connection pulley disposition region 6143 may be formed in one region of the body 6142, such as one region in a front side of the body 6142, specifically, one region of the body 6142 facing a distal end 6101d of the first jaw 6101. The connection pulley disposition region 6143 may be formed in various shapes, including, for example, a groove structure, which is open in a forward-facing direction, in one region of the body 6142.

A connection pulley 6129 may be disposed in the connection pulley disposition region 6143.

When the connection pulley 6129 is disposed in the connection pulley disposition region 6143, the connection pulley 6129 may be disposed in the connection pulley disposition region 6143 in a direction intersecting the body 6142, for example, in a direction orthogonal to a longitudinal direction of the body 6142. That is, in other words, a central axis of the connection pulley 6129 may be parallel to or coincide with the longitudinal direction of the body 6142.

Further, the connection pulley 6129 may be connected or fixed to the operation member 6140. For example, the central axis of the connection pulley 6129 may be connected or fixed to the operation member 6140. Accordingly, a driving force passing through the first forward-moving wire 6111 and the second forward-moving wire 6112 may be transmitted to the operation member 6140 through the connection pulley 6129.

The first forward-moving wire 6111 and the second forward-moving wire 6112 may be connected to the connection pulley 6129. This connection may be adaptable to various forms. As an example, the first forward-moving wire 6111 and the second forward-moving wire 6112 may have a structure of being wound around the connection pulley 6129 as a single wire, and based on FIG. 93, the left-wound region of the single wire may correspond to the first forward-moving wire 6111, while the right-wound region of the single wire may correspond to the second forward-moving wire 6112.

In addition, in an optional embodiment, it is also possible to have a structure in which the first forward-moving wire 6111 and the second forward-moving wire 6112 are formed as separate wires, with each end connected to the connection pulley 6129.

With the above configuration, when the first forward-moving wire 6111 and the second forward-moving wire 6112 are pulled, a force for pulling the first forward-moving wire 6111 and the second forward-moving wire 6112 is transmitted to the operation member 6140 through the connection pulley 6129, so that the operation member 6140 may move, i.e., move forward.

In an optional embodiment, when the connection pulley 6129 is disposed in the connection pulley disposition region 6143, the central axis of the connection pulley 6129 may be parallel to or coincide with a height direction of the body 6142. Accordingly, the connection pulley 6129 may be horizontally disposed, such as being perpendicular to the height direction of the operation member 6140. Accordingly, it is easy to implement a structure in which the first forward-moving wire 6111 and the second forward-moving wire 6112 are symmetrical to both sides around the operation member 6140.

In addition, in an optional embodiment, the connection pulley 6129 may be disposed below the first jaw 6101, that is, below the cartridge when the cartridge is disposed in the first jaw 6101, and one region of each of the first forward-moving wire 6111 and the second forward-moving wire 6112 may be easily disposed below the cartridge.

In an optional embodiment, a connection region for a backward-moving wire BRW may be formed in a rear side region of the body 6142.

FIGS. 95 and 96A, 96B, and 96C are schematic views for describing an operation of the operation member of the end tool of FIG. 90.

Referring to FIGS. 95 and 96A, 96B, and 96C, for convenience of description, the first jaw 6101 is excluded, and the first forward-moving wire 6111, the second forward-moving wire 6112, the first fixed pulley 6121, the second fixed pulley 6122, and the operation member 6140 are illustrated.

Based on FIG. 95, the operation member 6140 may move in a leftward direction, i.e., move forward toward the distal end 6101d, and this forward movement is illustrated sequentially in FIGS. 96A, 96B, and 96C.

As shown in FIG. 96A, when the first forward-moving wire 6111 and the second forward-moving wire 6112 are pulled in a first direction D1, one region of each of the first forward-moving wire 6111 and the second forward-moving wire 6112 is pulled in the first direction D1, and thus the regions of the first forward-moving wire 6111 and the second forward-moving wire 6112 emerging from the lower sides of the first fixed pulley 6121 and the second fixed pulley 6122 after being wound around the upper sides thereof move in a second direction D2, which is the opposite direction of the first direction D1. Accordingly, a driving force of each of the first forward-moving wire 6111 and the second forward-moving wire 6112 is transmitted to the operation member 6140 through the connection pulley 6129, and the force causes the operation member 6140 to move in a direction K1, which is the same direction as the second direction D2, i.e., move forward, thereby positioning the operation member 6140 in an advanced position shown in FIG. 96B.

Thereafter, as shown in FIG. 96B, when the first forward-moving wire 6111 and the second forward-moving wire 6112 are pulled further in the first direction D1, one region of the first forward-moving wire 6111 and the second forward-moving wire 6112 is pulled further in the first direction D1, and thus the regions of the first forward-moving wire 6111 and the second forward-moving wire 6112 emerging from the lower sides of the first fixed pulley 6121 and the second fixed pulley 6122 after being wound around the upper sides thereof move further in the second direction D2, which is the opposite direction of the first direction D1. Accordingly, a driving force of each of the first forward-moving wire 6111 and the second forward-moving wire 6112 is transmitted to the operation member 6140 through the connection pulley 6129, and the force causes the operation member 6140 to move further in the direction K1, which is the same direction as the second direction D2, i.e., move forward to a further advanced position than in FIG. 96B, thereby positioning the operation member 6140 in the further advanced position shown in FIG. 96C.

FIGS. 97 and 98A, 98B, and 98C are views for describing an optional embodiment in which a backward-moving wire is added to the end tool of FIG. 90.

Referring to FIGS. 97 and 98A, 98B, and 98C, the end tool of the present embodiment may further include the backward-moving wire BRW.

Referring to FIGS. 97 and 98A, 98B, and 98C, for convenience of description, the first jaw 6101 is excluded, and the first forward-moving wire 6111, the second forward-moving wire 6112, the first fixed pulley 6121, the second fixed pulley 6122, the operation member 6140, and the backward-moving wire BRW are illustrated.

Based on FIG. 97, the operation member 6140 may move in a rightward direction, i.e., move backward toward a proximal end 6101p, and this backward movement is illustrated sequentially in FIGS. 98A, 98B, and 98C.

The backward-moving wire BRW may be connected to one region of the operation member 6140 and may be connected, for example, to a rear side of the operation member, specifically, to a region of the blade region 6142a, which is opposite to the region in which the edge of the blade region 6142a is formed, among regions of the body 6142. The backward-moving wire BRW may be disposed to be placed between the connection pulley 6129 and a region of the first forward-moving wire and the second forward-moving wire 6111 and 6112 that extends to the proximal end of the jaw after passing through the operation member 6140.

A driving part or driving transmission part (e.g., a wire, a pulley, or the like) capable of pulling the backward-moving wire BRW may be connected to the backward-moving wire BRW, and the backward-moving wire BRW may be operated according to manual or automatic manipulation. For example, the backward-moving wire BRW may be pulled by the operator (refer to, for example, 3200 of FIG. 50).

By pulling the backward-moving wire BRW. The operation member 6140 may move backward.

For example, as shown in FIG. 98A, the backward-moving wire BRW is pulled in a reverse direction B1 in a state in which the operation member 6140 is located adjacent to the distal end 6101d of the first jaw 6101, the operation member 6140 connected to the backward-moving wire BRW moves backward in the direction K2, which is the same direction as the reverse direction B1.

At this point, the first forward-moving wire 6111 and the second forward-moving wire 6112 may be in a state in which a driving force, i.e., a pulling force is not applied.

When the operation member 6140 moves backward (in the direction K2), the region of the first forward-moving wire 6111 and the second forward-moving wire 6112 directed toward the first fixed pulley 6121 and the second fixed pulley 6122 from the connection pulley 6129 may move in the first direction D1, which is the same direction as the direction K2, and the regions of the first forward-moving wire 6111 and the second forward-moving wire 6112, which are wound around the lower sides of the first fixed pulley 6121 and the second fixed pulley 6122 and disposed on the upper sides thereof, may move in the second direction D2, which is the opposite direction of the direction K2. Accordingly, the operation member 6140 is in a position as shown in FIG. 98B, having moved backward to be closer to the proximal end 6101p compared to its position in FIG. 98A.

Thereafter, as shown in FIG. 98B, when the backward-moving wire BRW is pulled further in the reverse direction B1, the operation member 6140 connected to the backward-moving wire BRW moves backward in the direction K2, which is the same direction as the reverse direction B1. At this point, the first forward-moving wire 6111 and the second forward-moving wire 6112 may be in a state in which a pulling force is not applied. The region of the first forward-moving wire 6111 and the second forward-moving wire 6112 directed toward the first fixed pulley 6121 and the second fixed pulley 6122 from the connection pulley 6129 moves in the first direction D1, which is the same direction as the direction K2, and the regions of the first forward-moving wire 6111 and the second forward-moving wire 6112, which are wound around the lower sides of the first fixed pulley 6121 and the second fixed pulley 6122 and disposed on the upper sides thereof, may move in the second direction D2, which is the opposite direction of the direction K2. Accordingly, the operation member 6140 is in a position as shown in FIG. 98C, having moved backward to be closer to the proximal end 6101p compared to its position in FIG. 98B.

In the case of the present embodiment, as an example, the first forward-moving wire 6111 and the second forward-moving wire 6112 are connected to the connection pulley 6129 instead of being directly connected to or fixed to the operation member 6140, and the connection pulley 6129 is connected to the operation member 6140, thereby transmitting the driving force of the first forward-moving wire 6111 and the second forward-moving wire 6112 to the operation member 6140 through the connection pulley 6129.

With this configuration, damage or deformation of the first forward-moving wire 6111 and the second forward-moving wire 6112 may be reduced, thereby improving durability and performing stable driving. For example, if one of the first forward-moving wire 6111 and the second forward-moving wire 6112 are directly fixed to the operation member 6140, when one of the first forward-moving wire 6111 and the second forward-moving wire 6112 is pulled more than another one, the operation member 6140 may be twisted and deformed in position, and tension may be concentrated on the wire that is pulled more than another one, increasing the risk of breakage or disconnection. But, in the present embodiment, the first forward-moving wire 6111 and the second forward-moving wire 6112 are connected to the operation member 6140 through the connection pulley 6129, and thus, even when one of the two wires is pulled more than another one, the tolerance between the first forward-moving wire 6111 and the second forward-moving wire 6112 may be compensated for by a rotational motion of the connection pulley 6129.

In other words, even when a greater force is applied to one of the first forward-moving wire 6111 and the second forward-moving wire 6112, the connection pulley 6129 may rotate in a direction that temporarily releases the wire under more severe tension to relieve the tension acting on one wire, thereby reducing or preventing an imbalance in the tension of the first forward-moving wire 6111 and the second forward-moving wire 6112.

Meanwhile, when the first forward-moving wire 6111 and the second forward-moving wire 6112 are wound in opposite directions around the yaw pulley shaft or the pitch pulley shaft, during a yaw motion or a pitch motion of the end tool, one of the first forward-moving wire 6111 and the second forward-moving wire 6112 is wound, and another one is unwound. In this case, of the first forward-moving wire 6111 and the second forward-moving wire 6112, the wound wire causes the operation member 6140 to move forward toward the distal end, while the unwound wire has reduced or lost tension. At this point, in the case of the structure of the present embodiment, by facilitating the configuration of the first forward-moving wire 6111 and the second forward-moving wire 6112 into a single loop, the total length of the loop is maintained even when one of the first forward-moving wire 6111 and the second forward-moving wire 6112 is wound and another one is unwound during a yaw or pitch motion of the end tool, thereby reducing or preventing tension reduction or tension loss.

FIG. 99 is a perspective view illustrating one modified example of the operation member of FIG. 59. FIG. 100 is a perspective view illustrating the operation member in a direction different from that of FIG. 99. FIG. 101 is a perspective view illustrating a state in which a forward-moving wire and a backward-moving wire are connected to the operation member of FIG. 99.

An operation member 7140 may include a body 7142, a first clamp 7146, and a second clamp 7147. Meanwhile, the operation member 7140 may be used together with a wedge (refer to WDG of FIGS. 54 and 55), and for example, the wedge WDG may be prepared separately from the operation member 7140 and then disposed adjacent to the operation member 7140. As another example, the operation member 7140 and the wedge WDG may be integrally formed. In the present specification, for convenience of description, the operation member 7140 and the wedge WDG will be described and illustrated in the drawings with the assumption that the operation member 7140 and the wedge WDG are prepared separately.

The body 7142 may be in the form of an elongated column, such as a plate-shaped column. In addition, a blade region 7142a may be formed in one region of the body 7142, and an edge sharply formed to cut tissue may be formed in the blade region 7142a.

The first clamp 7146 may be formed in one region of the body 7142, and the second clamp 7147 may be formed in another region different from the one region. For example, the body 7142 may be disposed between the first clamp 7146 and the second clamp 7147. The configuration of the first clamp 7146 and the second clamp 7147 and the effects thereof are substantially the same as those in the previously described embodiments.

A first connection region 7143p1 and a second connection region 7143p2 may be formed in one region of the second clamp 7147, such as one region of an upper surface of the second clamp 7147.

For example, the first connection region 7143p1 and the second connection region 7143p2 may be disposed in one region of the upper surface of the second clamp 7147 to be spaced apart from the body 7142 and closer to a distal end (refer to, for example, 3101d of FIG. 58) of the first jaw.

As a specific example, a protruding region 7143 is formed in one region of the upper surface of the second clamp 7147, and the first connection region 7143p1 and the second connection region 7143p2 may be formed in the protruding region 7143. As an example, the first connection region 7143p1 and the second connection region 7143p2 may each have a through-hole shape passing through the protruding region 7143.

The first connection region 7143p1 and the second connection region 7143p2 may be regions to which a first forward-moving wire 7111 and a second forward-moving wire 7112 are connected, respectively, and may each be in the form of a fixing groove such that respective one end portion region of the first forward-moving wire 7111 and the second forward-moving wire 7112 is accommodated or fixed thereto.

In an optional embodiment, there may be a region in which a crimp is formed that is fixed after the first forward-moving wire 7111 and the second forward-moving wire 7112 pass through the through-hole shaped first connection region 7143p1 and the second connection region 7143p2, and a crimp groove 7147a may be formed at a position overlapping the region in which the crimp is formed. As an example, the crimp groove 7147a may be formed to have a depth of a set thickness on one side of the upper surface of the second clamp 7147 to be closer to a proximal end of the jaw than the protruding region 7143, or between the protruding region 7143 and the body 7142. The crimp groove 7147a may facilitate the region in which the crimp is formed to reduce or prevent interference of the crimp with one region of the operation member 7140.

When the first forward-moving wire 7111 and the second forward-moving wire 7112 are connected to the first connection region 7143p1 and the second connection region 7143p2 to pull the first forward-moving wire 7111 and the second forward-moving wire 7112, respectively, forces pulling the first forward-moving wire 7111 and the second forward-moving wire 7112 are transmitted to the operation member 7140 through the first connection region 7143p1 and the second connection region 7143p2 so that the operation member 7140 may move, i.e., move forward. By forming the first connection region 7143p1 and the second connection region 7143p2 below the body 7142, that is, in the second clamp 7147, when the first forward-moving wire 7111 and the second forward-moving wire 7112 are pulled, the pulling force is more stably transmitted to the operation member 7140 through the first connection region 7143p1 and the second connection region 7143p2. In addition, by disposing the first connection region 7143p1 and the second connection region 7143p2 closer to the front of the body 7142, that is, closer to the distal end of the jaw than the body 7142, the force pulling the operation member 7140 can be transmitted more efficiently, a moving direction of the operation member 7140 when moving forward can be precisely controlled, and symmetrical distribution of forces of the first connection region 7143p1 and the second connection region 7143p2 can be easily implemented.

In an optional embodiment, a connection region for the backward-moving wire may be formed in a rear side region of the body 7142.

For example, one or more backward-moving wire connection regions 7142b1 and 7142b2 and one or more crimp spaces 7142cl and 7142c2 may be formed in a rear side of the body 7142, i.e., in a surface of the body 7142, which is opposite to the surface in which the blade region 7142a is formed.

As a specific example, two backward-moving wire connection regions 7142b1 and 7142b2 and two crimp spaces 7142cl and 7142c2 may be formed, in this case, when one backward-moving wire BRW is disposed as illustrated in FIG. 101, one of the two backward-moving wire connection regions 7142b1 and 7142b2 and the two crimp spaces 7142cl and 7142c2, which are of different heights, may be selected depending on the specific structure and material of the operation member 7140 and the jaw or the end tool including the same.

Further, as another example, when controlling backward movement through two backward-moving wires, the two backward-moving wires may be connected to the two backward-moving wire connection regions 7142b1 and 7142b2 and the two crimp spaces 7142cl and 7142c2, respectively.

The backward-moving wire connection regions 7142b1 and 7142b2 may have various shapes to connect the backward-moving wire BRW thereto, and for example, the backward-moving wire connection regions 7142b1 and 7142b2 may have a through-hole shape so that the backward-moving wire BRW may pass therethrough. At this point, the one or more crimp spaces 7142cl and 7142c2 may be formed by removing one region from the body 7142 to have a space shape connected to the through-hole shape, and the one or more crimp spaces 7142cl and 7142c2 may have a region in which a crimp to which the backward-moving wire BRW is fixed. With such a structure, one or more backward-moving wires BRW may be easily connected to the operation member 7140, and a space for arranging the backward-moving wires BRW may be efficiently secured.

FIG. 102 is a perspective view schematically illustrating a surgical instrument according to another embodiment of the present disclosure. FIG. 103 is a side view of the surgical instrument of FIG. 102. FIGS. 104A and 104B are diagrams for schematically describing an operation concept of the surgical instrument of FIG. 102.

Referring to FIGS. 102 and 103, a surgical instrument 5000 according to the present embodiment may include an end tool 3100, an operator 5200, an operating force transmitter (not shown), and a connector 5400.

Since the end tool 3100 may include the end tool 3100 in the embodiment described above with reference to FIG. 50 without change, a detailed description thereof will be omitted. In addition, although not shown in the drawings, in an optional embodiment, it will be appreciated of course that one of the end tools 1100, 2100, 3100, 4100, and 6100 may be optionally applied to the surgical instrument 5000, and the above-described modified example may be optionally applied to the surgical instrument 5000. The end tool 3100 is illustrated only for convenience of description,

The connector 5400 is formed in the shape of a hollow shaft, and one or more wires and electric wires may be accommodated therein. The operator 5200 is coupled to one end portion of the connector 5400, the end tool 3100 is coupled to another end portion thereof, and the connector 5400 may serve to connect the operator 5200 to the end tool 3100.

In an optional embodiment, the connector 5400 includes a straight part 5401 and a bent part 5402. The straight part 5401 may be formed on a side of the connector 5400 coupled to the end tool 3100, and the bent part 5402 may be formed on a side of the connector 5400 to which the operator 5200 is coupled. As such, since the end portion of the connector 5400 at the side of the operator 5200 is formed to be bent, a pitch manipulation part 5201, a yaw manipulation part 5202, and an actuation manipulation part 5203 may be formed along an extension line of the end tool 3100 or adjacent to the extension line. In other words, it may be said that the pitch manipulation part 5201 and the yaw manipulation part 5202 are at least partially accommodated in a concave portion formed by the bent part 5402. Due to the above-described shape of the bent part 5402, the shapes and motions of the operator 5200 and the end tool 3100 may be further intuitively matched with each other.

Meanwhile, a plane on which the bent part 5402 is formed may be substantially the same as a pitch plane, that is, an XZ plane of FIG. 102. As such, as the bent part 5402 is formed on substantially the same plane as the XZ plane, interference with the operator may be reduced. Of course, for intuitive motions of the end tool and the operator, any form other than the XZ plane may be possible.

Meanwhile, in an optional embodiment, a connector 5410 may be formed in the bent part 5402. The connector 5410 may be connected to an external power source (not shown), and the connector 5410 may also be connected to the end tool 3100 via an electric wire, and may transmit, to the end tool 3100, electric energy supplied from the external power source (not shown).

The operator 5200 is formed at the one end portion of the connector 5400 and provided as an interface to be directly controlled by a medical doctor, such as, a handle 5204, a tongs shape, a stick shape, a lever shape, or the like, and when the medical doctor controls the operator 5200, the end tool 3100, which is connected to the interface and inserted into the body of a surgical patient, performs a certain motion, thereby performing surgery. Here, the operator 5200 is illustrated as being formed in a handle shape that is rotatable while the finger is inserted therein, but the concept of the present disclosure is not limited thereto, and various types of manipulation parts that can be connected to the end tool 3100 and manipulate the end tool 3100 may be possible. In addition, various controls of the operator 5200 may be possible through one or more driving shafts disposed in a direction from a proximal end 5206 of the operator 5200 to a distal end 5205 of the operator 5200.

Further, the operator 5200 of the surgical instrument 5000 may further include a staple manipulation part 5260 configured to perform stapling and cutting of the end tool 3100.

Meanwhile, the present embodiment may also be applicable to a robot-driven type rather than being limited to a manual type directly operated by a human operator. In this case, the end tool 3100 may be connected to the robot directly, or to a robot drive arm or other regions via the connector 5400, rather than being connected to the manual-type operator 5200.

The end tool 3100 of the surgical instrument 5000 according to the present embodiment is formed to be rotatable in at least one direction, and may be formed to perform a pitch motion around a Y-axis of FIG. 102 and, at the same time, perform a yaw motion and an actuation motion around a Z-axis.

Here, each of the pitch, yaw, and actuation motions used in FIG. 102 to FIGS. 104A and 104B are defined as follows.

First, the pitch motion means a motion of the end tool 3100 rotating in a vertical direction with respect to an extension direction of the connector 5400 (an X-axis direction of FIG. 102), that is, a motion rotating around the Y-axis of FIG. 102. In other words, the pitch motion means a motion of the end tool 3100, which is formed to extend from the connector 5400 in the extension direction of the connector 5400 (the X-axis direction of FIG. 102), rotating vertically around the Y-axis with respect to the connector 5400.

Next, the yaw motion means a motion of the end tool 3100 rotating in the left and right directions, that is, a motion rotating around the Z-axis of FIG. 102, with respect to the extension direction of the connector 5400 (the X-axis direction of FIG. 102). In other words, the yaw motion means a motion of the end tool 3100, which is formed to extend from the connector 5400 in the extension direction of the connector 5400 (the X-axis direction of FIG. 102), rotating horizontally around the Z-axis with respect to the connector 5400. That is, the yaw motion means a motion of the two jaws 3101 and 3102, which are formed on the end tool 3100, rotating around the Z-axis in the same direction.

Meanwhile, the actuation motion may mean a motion of the end tool 3100 rotating around the same axis of rotation as that of the yaw motion, while the two jaws 3101 and 3102 rotating in the opposite directions so as to be closed or opened. That is, the actuation motion means rotating motions of the two jaws 3101 and 3102, which are formed on the end tool 3100, in the opposite directions around the Z-axis.

The operating force transmitter (not shown) may connect the operator 5200 to the end tool 3100, transmit the driving force of the operator 5200 to the end tool 3100, and include a plurality of wires, pulleys, links, sections, gears, or the like.

FIGS. 104A and 104B illustrate an example of an operation of the surgical instrument 5000 of FIG. 102.

Specifically, referring to FIGS. 104A and 104B, in the surgical instrument, the end tool 3100 is formed in front of a rotation center 3100c of the end tool, and the operator 5200 is also formed in front of a rotation center 5200c of the operator so that motions of the operator 5200 and the end tool 3100 are intuitively matched. In other words, in the surgical instrument according to an embodiment of the present disclosure, at least a portion of the operator may become closer to the end tool than the joint of the operator in more than one moments during a manipulation process.

In the surgical instrument according to an embodiment of the present disclosure, since both the end tool and the operator are moved with respect to the rotation center formed at the rear side thereof, it may be said that the motions are intuitively matched with each other in terms of structure. In other words, moving portions of the operator are moved with respect to the rotation center formed at the rear side thereof just as moving portions of the end tool are moved with respect to the rotation center formed at the rear side thereof, and thus it may be said that the motions are intuitively matched with each other in terms of structure. This allows the user to intuitively and quickly perform a control in a direction toward the end tool, and a possibility of making a mistake may be reduced.

In an optional embodiment, a surgical instrument to which the end tool of the present embodiment is applied may be driven in various ways, for example, it is of course possible that, the surgical instrument is also applicable to embodiments in which the end tool and the operator are reversed around their central axes.

As described above, the present disclosure has been described with reference to the embodiment described with reference to the drawings, but it will be understood that this is merely exemplary, and those of ordinary skill in the art will understand that various modifications and other equivalent embodiments are possible therefrom. Accordingly, the true technical protection scope of the present disclosure should be defined by the technical spirit of the appended claims.

The embodiments may be represented by functional block elements and various processing steps. The functional blocks may be implemented as a varying number of hardware and/or software components that perform particular functions. For example, the embodiments may adopt integrated circuit configurations, such as memory, processing, logic, and/or look-up table, which may execute various functions by the control of one or more microprocessors or other control devices.

The particular implementations shown and described herein are illustrative examples of the embodiments and are not intended to otherwise limit the scope of the embodiments in any way. For the sake of brevity, conventional electronics, control methods, software development and other functional aspects of the methods may not be described in detail. Further, the connecting lines or connectors shown in the drawings are intended to represent example functional relationships and/or physical or logical couplings between the various elements. It should be noted that many alternative or additional functional relationships, physical connections, or logical connections may be present in a practical device. In addition, no item or element is essential to the practice of the present disclosure unless the element is specifically described as “essential” or “critical”.

The use of the terms “a” and “an” and “the” and similar referents in the context of describing the present disclosure (especially in the context of the following claims) are to be construed to cover both the singular and the plural. Further, recitation of ranges of values herein are merely intended to serve as a shorthand method of referring individually to each separate value falling within the range, unless otherwise indicated herein, and each separate value is incorporated into the specification as if it were individually recited herein. Finally, steps of all methods described herein can be performed in any suitable order unless otherwise indicated herein or otherwise clearly contradicted by context. The present disclosure is not necessarily limited to the described order of the steps. The use of any and all examples, or exemplary terms (e.g., “such as”) provided herein, is intended merely to better illuminate the present disclosure and does not pose a limitation on the scope of the present disclosure unless otherwise claimed. Further, numerous modifications and adaptations will be readily apparent to one of ordinary skill in the art without departing from the spirit and scope of the present disclosure.

An end tool and a surgical instrument according to the present disclosure can precisely move an operation member during one or more steps involved in performing laparoscopic surgery or various other surgeries.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An end tool comprising:
a jaw configured to accommodate at least one region of an operation member that is movable in at least one direction;
a fixed pulley disposed closer to a distal end of the jaw than the operation member; and
a forward-moving wire connected to the operation member,
wherein the forward-moving wire is configured to be rerouted in response to the fixed pulley to transmit a force to the operation member,
wherein at least one region of the forward-moving wire is configured to move toward a proximal end of the jaw to transmit a driving force to move the operation member forward toward the distal end of the jaw.

2. The end tool of claim 1,
wherein one region of the forward-moving wire is connected and fixed to one region of a front surface of the operation member facing a forward-moving direction, and
wherein another region of the forward-moving wire is disposed in a wire path that passes through front and rear sides of the operation member.

3. The end tool of claim 2, wherein the wire path, which is configured to pass therethrough the forward-moving wire, is defined in a body of the operation member at a side portion protruding from both sides of the body, the side portion having a predetermined width larger than the body of the operation member.

4. The end tool of claim 1,
wherein the fixed pulley is disposed to be inclined,
wherein one region of the forward-moving wire is connected to one region of a front surface of the operation member facing a forward-moving direction, and
wherein another region of the forward-moving wire is disposed at one outer side of a body of the operation member.

5. The end tool of claim 1,
wherein when the operation member moves forward,
one region of the forward-moving wire connected to the operation member and directed toward the fixed pulley is configured to move toward the distal end of the jaw, and
another region of the forward-moving wire, which is not fixed to the operation member and is directed to the fixed pulley, is configured to move toward the proximal end of the jaw.

6. An end tool comprising:
a jaw configured to accommodate at least one region of an operation member that is movable in at least one direction;
a fixed pulley disposed closer to a distal end of the jaw than the operation member; and
a forward-moving wire connected to a fixed region of the jaw,
wherein the forward-moving wire is configured to be rerouted in response to the fixed pulley and rerouted again by a support guide of the operation member,
wherein the forward-moving wire is further configured to transmit a force to the operation member and to move at least one region of the forward-moving wire toward a proximal end of the jaw to transmit a driving force to move the operation member forward toward the distal end of the jaw.

7. The end tool of claim 6, wherein the fixed region to which the forward-moving wire is fixed is disposed between the fixed pulley and the operation member.

8. The end tool of claim 7, wherein the fixed region is disposed between a region of the forward-moving wire directed to the fixed pulley and a region of the forward-moving wire directed to the support guide of the operation member from the fixed pulley.

9. The end tool of claim 8, wherein the forward-moving wire is configured to pass through a wire path, which passes through front and rear sides of the operation member, the forward-moving wire being directed toward the fixed pulley.

10. The end tool of claim 9,
wherein the support guide of the operation member is disposed on an inner side of the operation member, and
wherein after passing through the wire path and being directed toward the fixed pulley, the forward-moving wire is configured to be rerouted through the fixed pulley to enter a first path of the operation member and then emerge from a second path of the operation member after being correspondingly wound around the support guide to be fixed in the fixed region.

11. The end tool of claim 6, wherein the fixed region is disposed outside both a region of the forward-moving wire directed toward the fixed pulley and a region of the forward-moving wire directed toward the support guide of the operation member from the fixed pulley.

12. The end tool of claim 6,
wherein when the operation member moves forward,
one region of the forward-moving wire, which is wound around the support guide and directed toward the fixed pulley, is configured to move toward the distal end of the jaw, and
another region of the forward-moving wire, which does not correspond to the support guide and is directed to the fixed pulley, is configured to move toward the proximal end of the jaw.

13. The end tool of claim 1,
wherein the jaw includes a first jaw and a second jaw facing each other and configured to perform an opening and closing motion,
wherein the first jaw includes an accommodation part to accommodate a plurality of staples,
wherein the operation member includes a body, a blade region disposed in one region of the body, and a first clamp and a second clamp,
wherein each of the first clamp and the second clamp extends from the body and has a width greater than a width of the body, and
wherein the first clamp passes through a guide groove of the second jaw and corresponds to an upper surface of the second jaw, and the second clamp passes through a guide groove of the first jaw and corresponds to a lower surface of the first jaw.

14. The end tool of claim 13,
wherein the lower surface of the first jaw includes a protrusion and thus has a shape curved on both sides of the lower surface,
wherein the upper surface of the second jaw includes a protrusion and thus has a shape curved on both sides of the upper surface,
wherein the first clamp is curved to correspond to the protrusion of the second jaw, and
wherein the second clamp is curved to correspond to the protrusion of the first jaw.

15. The end tool of claim 1, further comprising a backward-moving wire connected to the operation member to enable the operation member to move backward in a direction opposite to a direction in which the operation member moves forward.

16. The end tool of claim 15, wherein the backward-moving wire includes a portion disposed in parallel to the forward-moving wire with respect to the operation member in a vertical direction.

17. The end tool of claim 15, wherein the backward-moving wire is disposed on a same line as one region of the forward-moving wire extending from the operation member.

18. An end tool comprising:
a jaw configured to accommodate at least one region of an operation member that is movable in at least one direction;
a plurality of fixed pulleys disposed closer to a distal end of the jaw than the operation member, the plurality of fixed pulleys being spaced apart from each other; and
a plurality of forward-moving wires which are connected to the operation member,
wherein the plurality of forward-moving wires are configured to be rerouted in response to the plurality of fixed pulleys, respectively, to transmit a force to the operation member,
wherein at least one region of each of the plurality of forward-moving wires is configured to move toward a proximal end of the jaw to transmit a driving force to move the operation member forward toward the distal end of the jaw.

19. The end tool of claim 18,
wherein the plurality of forward-moving wires include a first forward-moving wire and a second forward-moving wire, and
wherein the plurality of fixed pulleys include a first fixed pulley and a second fixed pulley that are spaced apart from each other to correspond to the first forward-moving wire and the second forward-moving wire, respectively.

20. The end tool of claim 19, wherein the first fixed pulley and the second fixed pulley are each inclined at an angle.

21. The end tool of claim 20, wherein the angle defined by the first fixed pulley and the second fixed pulley ranges from 70 degrees (deg) to 102 degrees (deg).

22. The end tool of claim 19, wherein a rotation shaft of the first fixed pulley and a rotation shaft of the second fixed pulley are disposed not to be parallel to each other.

23. The end tool of claim 20, wherein the first fixed pulley and the second fixed pulley have a gap therebetween that decreases in a downward direction with respect to a thickness direction of the jaw.

24. The end tool of claim 23,
wherein the jaw includes a first side surface and a second side surface, which have an inclined shape and face each other, and
wherein the first fixed pulley and the second fixed pulley are disposed on the first side surface and the second side surface of the jaw, respectively.

25. The end tool of claim 24, wherein the first fixed pulley and the second fixed pulley are disposed on the first side surface and the second side surface of the jaw, respectively, to be symmetrical to each other with respect to one center line of the jaw.

26. The end tool of claim 19, wherein the first forward-moving wire and the second forward-moving wire are connected to a first connection region and a second connection region, respectively, which are defined in the operation member at a region protruding outward from at least one surface.

27. The end tool of claim 26,
wherein the first forward-moving wire and the second forward-moving wire extend from the proximal end of the jaw toward the distal end of the jaw, emerge from lower sides of the first fixed pulley and the second fixed pulley while being wound around upper sides of the first fixed pulley and the second fixed pulley, and are connected to the first connection region and the second connection region, respectively.

28. The end tool of claim 19,
wherein a connection pulley is connected to the operation member and configured to move together with the operation member, and
wherein each of the first forward-moving wire and the second forward-moving wire is connected to the connection pulley.

29. The end tool of claim 28, wherein a central axis of the connection pulley is corresponding to or parallel to a height direction of a body of the operation member.

30. The end tool of claim 28,
wherein the connection pulley is disposed in a groove defined on one surface of a body of the operation member, and
wherein, when a cartridge is disposed in the jaw, the connection pulley is disposed between a lower side surface of the cartridge and a bottom of the jaw.

31. The end tool of claim 19,
wherein the jaw includes a first jaw and a second jaw facing each other and configured to perform an opening and closing motion,
wherein the first jaw includes an accommodation part configured to accommodate a cartridge having a plurality of staples, and
wherein the first fixed pulley and the second fixed pulley are disposed in a front space that is positioned closer to a distal end of the first jaw than the accommodation part of the first jaw.

32. The end tool of claim 31, wherein the first fixed pulley and the second fixed pulley are respectively disposed on a first side surface and a second side surface of the first jaw, which correspond to the front space, the first side surface and the second side surface having a gap therebetween decreasing in a direction away from the second jaw.

33. The end tool of claim 19, further comprising a plurality of switching pulleys disposed closer to the proximal end of the jaw than the operation member to control paths of the first forward-moving wire and the second forward-moving wire.

34. The end tool of claim 33,
wherein the plurality of switching pulleys include a first switching pulley configured to control the path of the first forward-moving wire and a second switching pulley configured to control the path of the second forward-moving wire, and
wherein the first switching pulley and the second switching pulley are disposed around switching pulley shafts, respectively, that are parallel to each other.

35. The end tool of claim 34, wherein the first switching pulley and the second switching pulley are disposed at positions offset from each other rather than aligned on a same line with respect to a direction toward the operation member.

36. The end tool of claim 33,
wherein the plurality of switching pulleys include a first plurality of switching pulleys configured to control the path of the first forward-moving wire and a second plurality of switching pulleys configured to control the path of the second forward-moving wire, and
wherein the first plurality of switching pulleys configured to control the path of the first forward-moving wire and the second plurality of switching pulleys configured to control the path of the second forward-moving wire are disposed on different switching pulley shafts.

37. The end tool of claim 36,
wherein the first plurality of switching pulleys configured to control the path of the first forward-moving wire include a third switching pulley and a fourth switching pulley disposed around first switching pulley shafts that are parallel to each other, and
wherein the second plurality of switching pulleys configured to control the path of the second forward-moving wire include a first switching pulley and a second switching pulley disposed around second switching pulley shafts that are parallel to each other.

38. The end tool of claim 37,
wherein the jaw includes a first side surface and a second side surface corresponding to a rear space that is defined closer to the proximal end of the jaw than the operation member,
wherein the third switching pulley and the fourth switching pulley configured to control the path of the first forward-moving wire are disposed on the first side surface of the rear space, and
wherein the first switching pulley and the second switching pulley configured to control the path of the second forward-moving wire are disposed on the second side surface of the rear space.

39. The end tool of claim 38, wherein the first side surface and the second side surface of the jaw have a gap therebetween that decreases in a downward direction with respect to a thickness direction of the jaw.

40. The end tool of claim 39,
wherein the third switching pulley and the fourth switching pulley, which are disposed on the first side surface and configured to control the path of the first forward-moving wire, are positioned at different heights, respectively, and
wherein the first switching pulley and the second switching pulley, which are disposed on the second side surface and configured to control the path of the second forward-moving wire, are positioned at different heights, respectively.

41. The end tool of claim 38, further comprising:
a backward-moving wire connected to the operation member to control a backward movement of the operation member; and
a backward-moving wire switching pulley corresponding to a support shaft passing through the first side surface and the second side surface, the backward-moving wire switching pulley being configured to control a path of the backward-moving wire.

42. The end tool of claim 41, wherein the backward-moving wire switching pulley is disposed between the first side surface and the second side surface to be spaced apart from the first side surface and the second side surface.

43. The end tool of claim 33, wherein the plurality of switching pulleys are disposed closer to the operation member than a rotational motion shaft configured to perform one or more joint motions of the end tool.

44. The end tool of claim 43, wherein the one or more joint motions includes a pitch motion or a yaw motion.

45. The end tool of claim 18,
wherein the plurality of forward-moving wires include a first forward-moving wire and a second forward-moving wire, and
wherein, when a cartridge is disposed in the jaw, at least one region of the first forward-moving wire and the second forward-moving wire includes regions that respectively correspond to spaces between both side surfaces of the jaw and the cartridge.

46. The end tool of claim 45,
wherein one region of the first forward-moving wire and one region of the second forward-moving wire emerging from being wound around a lower side of the plurality of fixed pulleys, respectively, are disposed on a lower side of the cartridge, and
wherein another region of the first forward-moving wire and another region the second forward-moving wire emerging from being wound around an upper side of the plurality of fixed pulleys, are disposed on both sides between the jaw and the cartridge, respectively.

47. The end tool of claim 18,
wherein when the operation member moves forward, one region of each of the plurality of forward-moving wires, which is connected to the operation member and directed toward each of the plurality of fixed pulleys, is configured to move toward the distal end of the jaw, and
wherein another region of each of the plurality of forward-moving wires, which is not fixed to the operation member and is directed to each of the plurality of fixed pulleys, is configured to move toward the proximal end of the jaw.

48. An end tool comprising:
a jaw configured to accommodate at least one region of an operation member that is movable in at least one direction;
a first rotation shaft pulley disposed to allow the jaw to rotationally move around one shaft;
a second rotation shaft pulley disposed to allow the jaw to rotationally move around another shaft that is different from the one shaft around which the first rotation shaft pulley rotationally moves; and
one or more switching pulleys configured to control one or more paths of one or more forward-moving wires that are configured to transmit a driving force to cause the operation member to move, and the one or more switching pulleys being disposed closer to the operation member than at least the first rotation shaft pulley and the second rotation shaft pulley.

49. The end tool of claim 48,
wherein the first rotation shaft pulley is a yaw pulley configured to correspond to a yaw motion of the end tool,
wherein the second rotation shaft pulley is a pitch pulley configured to correspond to a pitch motion of the end tool, and
wherein the first rotation shaft pulley is disposed closer to the one or more switching pulleys than the second rotation shaft pulley.

50. The end tool of claim 49,
wherein the one or more forward-moving wires include a first forward-moving wire and a second forward-moving wire,
wherein the one or more switching pulleys include a first switching pulley and a second switching pulley corresponding to the first forward-moving wire and the second forward-moving wire, respectively, and
wherein the first switching pulley and the second switching pulley are disposed parallel to each other.

51. The end tool of claim 50, further comprising:
a first auxiliary pitch pulley disposed adjacent to the pitch pulley, which is the second rotation shaft pulley,
wherein the first auxiliary pitch pulley has an axis parallel to an axis of the second rotation shaft pulley, the first auxiliary pitch pulley being configured to control paths of the first forward-moving wire and the second forward-moving wire.

52. The end tool of claim 51, further comprising:
a yaw auxiliary pitch pulley disposed between the first auxiliary pitch pulley and the first rotation shaft pulley,
wherein the yaw auxiliary pitch pulley has an axis parallel to an axis of the first rotation shaft pulley, the yaw auxiliary pitch pulley being configured to control the paths of the first forward-moving wire and the second forward-moving wire.

53. The end tool of claim 52, further comprising:
a second auxiliary pitch pulley disposed on a side of the pitch pulley, which is opposite to a side of the pitch pulley in which the first auxiliary pitch pulley is disposed,
wherein the second auxiliary pitch pulley has an axis parallel to the axis of the second rotation shaft pulley, the second auxiliary pitch pulley being configured to control the paths of the first forward-moving wire and the second forward-moving wire, wherein the pitch pulley is the second rotation shaft pulley.

54. The end tool of claim 50, wherein paths of the first forward-moving wire and the second forward-moving wire are configured to be controlled through the first switching pulley and the second switching pulley to gather the first forward-moving wire and the second forward-moving wire to be correspondingly in contact with a same side of at least the first rotation shaft pulley.

55. The end tool of claim 50, further comprising:
a backward movement wire for backward movement of the operation member,
wherein one or more backward-moving wire switching pulleys configured to control a path of the backward movement wire are disposed on a shaft of the first switching pulley or the second switching pulley.

56. The end tool of claim 48,
wherein the first rotation shaft pulley is a pitch pulley configured to correspond to a pitch motion of the end tool,
wherein the second rotation shaft pulley is a yaw pulley configured to correspond to a yaw motion of the end tool, and
wherein the first rotation shaft pulley is disposed closer to the one or more switching pulleys than the second rotation shaft pulley.

57. The end tool of claim 56,
wherein the one or more forward-moving wires include a first forward-moving wire and a second forward-moving wire, and
wherein the one or more switching pulleys include a third switching pulley and a fourth switching pulley, which correspond to the first forward-moving wire, and a first switching pulley and a second switching pulley, which correspond to the second forward-moving wire.

58. The end tool of claim 57,
wherein a central axis corresponding to the third switching pulley and a central axis corresponding to the fourth switching pulley are parallel to each other,
wherein a central axis corresponding to the first switching pulley and a central axis corresponding to the second switching pulley are parallel to each other, and
wherein the central axis corresponding to the third switching pulley and the central axis corresponding to the fourth switching pulley respectively intersect the central axis corresponding to the first switching pulley and the central axis corresponding to the second switching pulley.

59. The end tool of claim 58,
wherein the third switching pulley and the fourth switching pulley are disposed on a first plane,
wherein the first switching pulley and the second switching pulley are disposed on a second plane, and
wherein the first plane on which the third switching pulley and the fourth switching pulley are disposed and the second plane on which the first switching pulley and the second switching pulley are disposed, are inclined to each other.

60. The end tool of claim 59,
wherein the third switching pulley and the fourth switching pulley are positioned at different heights, respectively, and
wherein the first switching pulley and the second switching pulley are positioned at different heights, respectively.

61. The end tool of claim 60, wherein the third switching pulley and the fourth switching pulley are disposed to be symmetrical to the first switching pulley and the second switching pulley.

62. The end tool of claim 57, further comprising:
an auxiliary pitch pulley disposed adjacent to the pitch pulley, which is the first rotation shaft pulley,
wherein the auxiliary pitch pulley has an axis parallel to an axis of the first rotation shaft pulley, the auxiliary pitch pulley being configured to control paths of the first forward-moving wire and the second forward-moving wire.

63. The end tool of claim 62, further comprising:
a first auxiliary yaw pulley disposed between the auxiliary pitch pulley and the second rotation shaft pulley,
wherein the first auxiliary yaw pulley has an axis parallel to an axis of the second rotation shaft pulley, the first auxiliary yaw pulley being configured to control the paths of the first forward-moving wire and the second forward-moving wire.

64. The end tool of claim 63, further comprising:
a second auxiliary yaw pulley disposed on a side of the yaw pulley, which is opposite to a side of the yaw pulley in which the first auxiliary yaw pulley is disposed,
wherein the second auxiliary yaw pulley has an axis parallel to the axis of the second rotation shaft pulley, the second auxiliary yaw pulley being configured to control the paths of the first forward-moving wire and the second forward-moving wire, wherein the yaw pulley is the second rotation shaft pulley.

65. The end tool of claim 59, further comprising:
a backward-moving wire switching pulley disposed to correspond to a support shaft passing through the first plane and the second plane to control a path for backward movement of the operation member.

66. A surgical instrument comprising:
the end tool of claim 1;
an operator configured to control an operation of the end tool; and
a connector configured to connect the operator to the end tool.
